(19) United States
(12) Patent Application Publication
(10) Pub. No.: US 2022/0112175 A1
(43) Pub. Date: Apr. 14, 2022

(54) PYRIDAZINES HERBICIDAL COMPOUNDS

(71) Applicant: SYNGENTA CROP PROTECTION AG, Basel (CH)

(72) Inventors: Nigel James WILLETTS, Bracknell, Berkshire (GB); James Nicholas SCUTT, Bracknell, Berkshire (GB)

(73) Assignee: SYNGENTA CROP PROTECTION AG, Basel (CH)

(21) Appl. No.: 17/431,815

(22) PCT Filed: Feb. 18, 2020
(86) PCT No.: PCT/EP2020/054240

§ 371 Date: Aug. 18, 2021

(30) Foreign Application Priority Data
Feb. 21, 2019 (GB) 1902383.7

(51) Int. Cl. C07D 403/04; A01N 43/653; A01N 43/78; C07D 417/04; C07D 413/04; A01N 43/76; A01N 43/82; A01P 13/00

(52) U.S. Cl. CPC C07D 403/04; A01N 43/653; A01N 43/78; A01P 13/00; C07D 413/04; A01N 43/76; A01N 43/82; C07D 417/04

(57) ABSTRACT

Compounds of the formula (I) wherein the substituents are as defined in claim 1, useful as a pesticides, especially as herbicides.

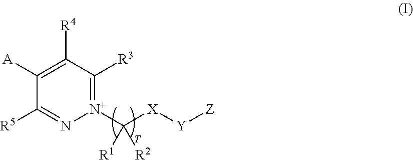

The present invention relates to herbicidally active pyridazine derivatives, as well as to processes and intermediates used for the preparation of such derivatives. The invention further extends to herbicidal compositions comprising such derivatives, as well as to the use of such compounds and compositions in controlling undesirable plant growth: in particular the use in controlling weeds, in crops of useful plants.

The present invention is based on the finding that pyridazine derivatives of formula (I) as defined herein, exhibit surprisingly good herbicidal activity. Thus, according to the present invention there is provided a compound of formula (I) or an agronomically acceptable salt or zwitterionic species thereof:

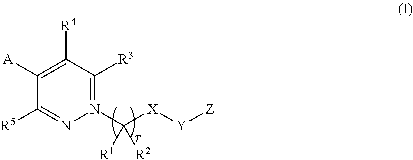

wherein,

T is 1, 2 or 3;

R¹ and R² are independently selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;

provided that when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, then the R² on the same carbon atom is selected from the group consisting of hydrogen and C₁-C₆alkyl; or

R¹ and R² together with the carbon atom to which they are attached form a C₃-C₆cycloalkyl ring or a 3- to 6-membered heterocyclyl, which comprises 1 or 2 heteroatoms individually selected from N and O;

Y is (CR^{1a}R^{2b})ₘ;

m is 1, 2 or 3;

each R^{1a} is independently selected from the group consisting of hydrogen, halogen, C₁-C₆alkyl, C₂-C₆alkenyl, C₂-C₆alkynyl, C₃-C₆cycloalkyl, C₁-C₆haloalkyl, —OH, —OR⁷, —OR^{15a}, —NH₂, —NHR⁷, —N(R⁷)₂, —NHR^{15a}, —NR^{7b}R^{7c}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂, —S(O)ᵣR¹⁵ and phenyl which is optionally substituted by 1, 2 or 3 R⁹ substituents, which may be the same or different, —C₁-C₆alkylNH₂, —C₁-C₆alkylNHR⁷, —C₁-C₆alkylN(R⁷)₂, —C₁-C₆alkylC(O)OR¹⁰, —C₁-C₆alkylOR¹⁰, —C₁-C₆alkylC(O)NR¹⁶R¹⁷, —C₁-C₆alkylSR¹⁰, —C₁-C₆alkylS(O)R¹⁰, —C₁-C₆alkylS(O)₂R¹⁰, —C₁-C₆NHC(═NH)NH₂, —C₁-C₃alkylphenyl, wherein said phenyl is optionally substituted by 1, 2 or 3 R⁹ substituents, which may be the same or different, and —C₁-C₃alkylheteroaromatic, wherein said heteroaromatic is a 5- to 10-membered cyclic or bicyclic aromatic ring which comprises 1, 2, 3 or 4 heteroatoms individually selected from nitrogen, oxygen and sulfur, optionally substituted by 1, 2 or 3 R⁹ substituents, which may be the same or different;

each R^{2b} is independently selected from the group consisting of hydrogen, halogen, C₁-C₆alkyl, C₁-C₆haloalkyl, —C₁-C₆alkylNH₂, —C₁-C₆alkylNHR⁷, —C₁-C₆alkylN(R⁷)₂, —C₁-C₆alkylC(O)OR¹⁰, —C₁-C₆alkylOR¹⁰, —C₁-C₆alkylC(O)NR¹⁶R¹⁷, —C₁-C₆alkylSR¹⁰, —C₁-C₆alkylS(O)R¹⁰, —C₁-C₆alkylS(O)₂R¹⁰, —C₁-C₆NHC(═NH)NH₂, —C₁-C₃alkylphenyl, wherein said phenyl is optionally substituted by 1, 2 or 3 R⁹ substituents, which may be the same or different, and —C₁-C₃alkylheteroaromatic, wherein said heteroaromatic is a 5- to 10-membered cyclic or bicyclic aromatic ring which comprises 1, 2, 3 or 4 heteroatoms individually selected from nitrogen, oxygen and sulfur, optionally substituted by 1, 2 or 3 R⁹ substituents, which may be the same or different; or

R^{1a} and R^{2b} together with the carbon atom to which they are attached form a C₃-C₆cycloalkyl ring or a 3- to 6-membered heterocyclyl, which comprises 1 or 2 heteroatoms individually selected from N and O;

R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;

each R⁶ is independently selected from hydrogen and C₁-C₆alkyl;

each R⁷ is independently selected from the group consisting of C₁-C₆alkyl, —S(O)₂R¹⁵, —C(O)R¹⁵, —C(O)OR¹⁵ and —C(O)NR¹⁶R¹⁷;

each R^{7a} is independently selected from the group consisting of —S(O)₂R¹⁵, —C(O)R¹⁵, —C(O)OR¹⁵, —C(O)NR¹⁶R¹⁷ and —C(O)NR¹⁶R^{15a};

R^{7b} and R^{7c} are independently selected from the group consisting of C₁-C₆alkyl, —S(O)ᵣR¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or

R^{7b} and R^{7c} together with the nitrogen atom to which they are attached form a 4- to 6-membered heterocyclyl ring which optionally comprises one additional heteroatom individually selected from N, O and S;

A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different,

and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different;

and/or when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy;

each R⁹ is independently selected from the group consisting of OH, halogen, cyano, —N(R⁶)₂, C₁-C₄alkyl, C₁-C₄alkoxy, C₁-C₄haloalkyl and C₁-C₄haloalkoxy;

X is selected from the group consisting of —C(O)—, —C(O)O—, —C(O)N(R⁴⁰)—, —C(O)N(R⁴²)O—, —C(O)N(R⁴⁰)N(R⁴⁰)—, —C(O)N(R⁴⁰)C(O)—, —C(O)N(R⁴⁰)C(O)N(R⁴⁰)—, —C(O)N(R⁴⁰)C(R⁴⁶)₂C(O)N(R⁴⁰)—, —C(O)N(R⁴⁰)C(R⁴⁶)₂C(O)N(R⁴⁰)C(R⁴⁶)₂C(O)N(R⁴⁰)—, —C(═NR⁴¹)—, —C(R⁴⁰)═NO—, —C(═NR⁴¹)N(R⁴⁰)—, —C(S)—, —C(S)N(R⁴⁰)—, —N(R⁴³)—, —N(R⁴²)O—, —N(R⁴³)N(R⁴³)—, —N(R⁴⁰)C(O)—, —N(R⁴⁰)C(S)—, —N(R⁴⁰)S(O)₂—, —N(R⁴⁰)C(O)O—, —N(R⁴⁰)P(O)(R⁴⁴)—, —N(R⁴⁰)P(O)(R⁴⁴)O—, —N(R⁴⁰)C(═NR⁴¹)—, —N(R⁴⁰)S(O)(═NR⁴⁰)—, —N(R⁴⁰)S(O)—, —N(R⁴⁰)C(O)S—, —N(R⁴⁰)C(O)N(R⁴⁰)—, —N(R⁴⁰)S(O)₂N(R⁴⁰)—, —N(R⁴⁰)C(S)N(R⁴⁰)—, —N(R⁴⁰)C(═NR⁴¹)N(R⁴⁰)—, —N(R⁴⁰)P(O)(R⁴⁴)N(R⁴⁰)—, —N(R⁴⁰)C(O)N(R⁴⁰)C(O)—, —N(R⁴⁰)N(R⁴⁰)C(O)—, —O—, —OC(O)—, —OC(O)O—, —OC(O)N(R⁴⁰)—, —ON(R⁴²)—, —ON═C(R⁴⁰)—, —ON(R⁴²)C(O)—, —OP(O)(R⁴⁴)—, —OP(O)(R⁴⁴)O—, —OP(O)(R⁴⁴)N(R⁴⁰)—, —OSi(R⁴⁰)₂—, —OSi(R⁴⁰)₂O—, —S—, —S(O)—, —S(O)₂—, —S(O)₂N(R⁴⁰)—, —SC(O)N(R⁴⁰)—, —S(O)N(R⁴⁰)—, —S(O)(═NR⁴⁰)—, —S(═NR⁴⁰)₂—, —S(O)(═NR⁴⁰)N(R⁴⁰)—, —S(═NR⁴⁰)—, —P(O)(R⁴⁴)—, —P(O)(R⁴⁴)N(R⁴⁰)—, —P(O)(R⁴⁴)O—, —C(═CR⁴⁵)₂—, —CR⁴⁵═CR⁴⁵— (E and Z isomers), —C≡C—, —Si(R⁴⁰)₂— and —Si(R⁴⁰)₂₀—;

R⁴⁰ is selected from the group consisting of hydrogen, C₁-C₆alkyl, C₁-C₆alkoxy, C₁-C₃alkoxyC₁-C₃alkyl;

R⁴¹ is selected from the group consisting of hydrogen, C₁-C₆alkyl, C₁-C₆alkoxy, C₁-C₆alkylamino, di-C₁-C₆alkylamino, cyano;

R⁴² is selected from the group consisting of hydrogen, C₁-C₆alkyl, C₁-C₆alkoxyC₁-C₃alkyl, C₁-C₆ alkylcarbonyl, C₁-C₆alkoxycarbonyl, C₁-C₆alkylsulfonyl;

R⁴³ is selected from the group consisting of hydrogen, C₁-C₆alkyl, C₁-C₆alkoxy, C₁-C₃alkoxyC₁-C₃alkyl, C₁-C₆alkylcarbonyl, C₁-C₆alkoxycarbonyl, and C₁-C₆alkylsulfonyl;

R⁴⁴ is selected from the group consisting of hydrogen, C₁-C₆alkyl, OH, C₁-C₆alkoxy, C₁-C₆alkoxyC₁-C₃alkyl, NH₂, and C₁-C₆alkylamino, di-C₁-C₆alkylamino,

R⁴⁵ is selected from the group consisting of hydrogen, halogen, and C₁-C₆alkyl;

R⁴⁶ is selected from the group consisting of hydrogen, C₁-C₆alkyl, C₁-C₆alkoxy, C₁-C₆alkoxyC₁-C₃alkyl, —C₁-C₆alkylNH₂, —C₁-C₆alkylNHR⁷, —C₁-C₆alkylN(R⁷)₂, —C₁-C₆alkylC(O)OR¹⁰, —C₁-C₆alkylOR¹⁰, —C₁-C₆alkylC(O)NR¹⁶R¹⁷, —C₁-C₆alkylSR¹⁰, —C₁-C₆alkylS(O)R¹⁰, —C₁-C₆alkylS(O)₂R¹⁰, —C₁-C₆NHC(═NH)NH₂, —C₁-C₃alkylC₁-C₃alkoxy, —C₁-C₃alkylphenyl, wherein said phenyl is optionally substituted by 1, 2 or 3 R⁹ substituents, which may be the same or different, and —C₁-C₃alkylheteroaromatic, wherein said heteroaromatic is a 5- to 10-membered cyclic or bicyclic aromatic ring which comprises 1, 2, 3 or 4 heteroatoms individually selected from nitrogen, oxygen and sulfur, optionally substituted by 1, 2 or 3 R⁹ substituents, which may be the same or different;

Z is selected from the group consisting of —C(O)OR¹⁰, —OH, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;

R¹¹ is selected from the group consisting of hydrogen, C₁-C₆alkyl and phenyl, and wherein said phenyl is optionally substituted by 1, 2 or 3 R⁹ substituents, which may be the same or different;

R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;

R¹³ is selected from the group consisting of —OH, C₁-C₆alkyl, C₁-C₆alkoxy and phenyl;

R¹⁴ is C₁-C₆haloalkyl;

R¹⁵ is selected from the group consisting of C₁-C₆alkyl and phenyl, and wherein said phenyl is optionally substituted by 1, 2 or 3 R⁹ substituents, which may be the same or different;

R^{15a} is phenyl, wherein said phenyl is optionally substituted by 1, 2 or 3 R⁹ substituents, which may be the same or different;

R¹⁶ and R¹⁷ are independently selected from the group consisting of hydrogen and C₁-C₆alkyl; or

R¹⁶ and R¹⁷ together with the nitrogen atom to which they are attached form a 4- to 6-membered heterocyclyl ring which optionally comprises one additional heteroatom individually selected from N, O and S;

R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different; and

r is 0, 1 or 2.

According to a second aspect of the invention, there is provided an agrochemical composition comprising a herbicidally effective amount of a compound of formula (I). Such an agricultural composition may further comprise at least one additional active ingredient and/or an agrochemically acceptable diluent or carrier.

According to a third aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

According to a fourth aspect of the invention, there is provided the use of a compound of formula (I) as a herbicide.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, nitro means an —NO₂ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl, ethyl, n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. C₁-C₄alkoxy is to be construed accordingly. Examples of C₁₋₄alkoxy include, but are not limited to, methoxy, ethoxy, propoxy, iso-propoxy and t-butoxy.

As used herein, the term “C₁-C₆haloalkyl” refers to a C₁-C₆alkyl radical as generally defined above substituted by one or more of the same or different halogen atoms. C₁-C₄haloalkyl is to be construed accordingly. Examples of C₁-C₆haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. C₂-C₄alkenyl is to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₂-C₄alkynyl is to be construed accordingly. Examples of C₂-C₆alkynyl include, but are not limited to, prop-1-ynyl, propargyl (prop-2-ynyl) and but-1-ynyl.

As used herein, the term “C₁-C₆haloalkoxy” refers to a C₁-C₆alkoxy group as defined above substituted by one or more of the same or different halogen atoms. C₁-C₄haloalkoxy is to be construed accordingly. Examples of C₁-C₆haloalkoxy include, but are not limited to, fluoromethoxy, difluoromethoxy, fluoroethoxy, trifluoromethoxy and trifluoroethoxy.

As used herein, the term “C₁-C₃haloalkoxyC₁-C₃alkyl” refers to a radical of the formula R_{b}—O—Rₐ— where R_{b} is a C₁-C₃haloalkyl radical as generally defined above, and Rₐ is a C₁-C₃alkylene radical as generally defined above.

As used herein, the term “C₁-C₃alkoxyC₁-C₃alkyl” refers to a radical of the formula R_{b}—O—Rₐ— where R_{b} is a C₁-C₃alkyl radical as generally defined above, and Rₐ is a C₁-C₃alkylene radical as generally defined above.

As used herein, the term “C₁-C₃alkoxyC₁-C₃alkoxy-” refers to a radical of the formula R_{b}—O—Rₐ—O— where R_{b} is a C₁-C₃alkyl radical as generally defined above, and Rₐ is a C₁-C₃alkylene radical as generally defined above.

As used herein, the term “C₃-C₆alkenyloxy” refers to a radical of the formula —ORₐ where Rₐ is a C₃-C₆alkenyl radical as generally defined above.

As used herein, the term “C₃-C₆alkynyloxy” refers to a radical of the formula —ORₐ where Rₐ is a C₃-C₆alkynyl radical as generally defined above.

As used herein, the term “hydroxyC₁-C₆alkyl” refers to a C₁-C₆alkyl radical as generally defined above substituted by one or more hydroxy groups.

As used herein, the term “C₁-C₆alkylcarbonyl” refers to a radical of the formula —C(O)Rₐ where Rₐ is a C₁-C₆alkyl radical as generally defined above.

As used herein, the term “C₁-C₆alkoxycarbonyl” refers to a radical of the formula —C(O)ORₐ where Rₐ is a C₁-C₆alkyl radical as generally defined above.

As used herein, the term “aminocarbonyl” refers to a radical of the formula —C(O)NH₂.

As used herein, the term “C₁-C₆alkylaminocarbonyl” refers to a radical of the formula —C(O)NHRₐ where Rₐ is a C₁-C₆alkyl radical as generally defined above.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₃-C₆halocycloalkyl” refers to a C₃-C₆cycloalkyl radical as generally defined above substituted by one or more of the same or different halogen atoms. C₃-C₄halocycloalkyl is to be construed accordingly.

As used herein, the term “C₃-C₆cycloalkoxy” refers to a radical of the formula —ORₐ where Rₐ is a C₃-C₆cycloalkyl radical as generally defined above.

As used herein, except where explicitly stated otherwise, the term “heteroaryl” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 4- to 6-membered non-aromatic monocyclic ring radical which comprises 1, 2, or 3 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) may exist as an agronomically acceptable salt, a zwitterion or an agronomically acceptable salt of a zwitterion. This invention covers all such agronomically acceptable salts, zwitterions and mixtures thereof in all proportions.

For example a compound of formula (I) wherein Z comprises an acidic proton, may exist as a zwitterion, a compound of formula (I-I), or as an agronomically acceptable salt of an acid, a compound of formula (I-II) as shown below:

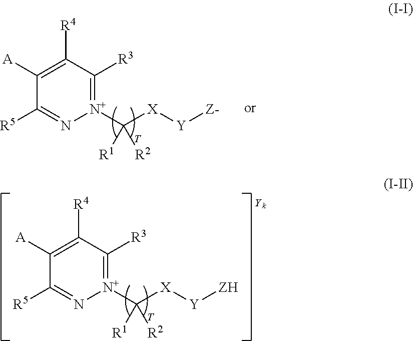

wherein, Y represents an agronomically acceptable anion and j and k represent integers that may be selected from 1, 2 or 3, dependant upon the charge of the respective anion Y.

A compound of formula (I) may also exist as an agronomically acceptable salt of a zwitterion, a compound of formula (I-III) as shown below:

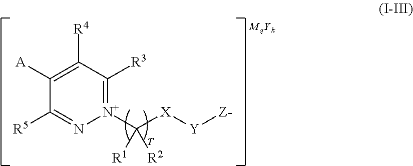

wherein, Y represents an agronomically acceptable anion, M represents an agronomically acceptable cation (in addition to the pyridazinium cation) and the integers j, k and q may be selected from 1, 2 or 3, dependant upon the charge of the respective anion Y and respective cation M.

Thus where a compound of formula (I) is drawn in protonated form herein, the skilled person would appreciate that it could equally be represented in unprotonated or salt form with one or more relevant counter ions.

In one embodiment of the invention there is provided a compound of formula (I-II) wherein k is 2, j is 1 and Y is selected from the group consisting of halogen, trifluoroacetate and pentafluoropropionate. In this embodiment a nitrogen atom in the aromatic ring A may be protonated or a nitrogen atom comprised in R¹, R², Q or X may be protonated. Preferably, in a compound of formula (I-II), k is 2, j is 1 and Y is chloride, wherein a nitrogen atom in the aromatic ring A is protonated.

Suitable agronomically acceptable salts of the present invention, represented by an anion Y, include but are not limited chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, perchlorate, phosphate, propionate, propylsulfate, propylsulfonate, succinate, sulfate, tartrate, tosylate, tridecylate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of formula (I), wherein Z comprises an acidic proton, can be represented as either (I-I) or (I-II). For compounds of formula (I-II) emphasis is given to salts when Y is chloride, bromide, iodide, hydroxide, bicarbonate, acetate, trifluoroacetate, methylsulfate, tosylate and nitrate, wherein j and k are 1. For compounds of formula (I-II) emphasis is also given to salts when Y is carbonate and sulfate, wherein j is 2 and k is 1, and when Y is phosphate, wherein j is 3 and k is 1.

Where appropriate compounds of formula (I) may also be in the form of (and/or be used as) an N-oxide.

The following list provides definitions, including preferred definitions, for substituents m, r, T, A, X, Z, R¹, R², R^{1a}, R^{2b}, R³, R⁴, R⁵, R⁶, R⁷, R^{7a}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ with reference to the compounds of formula (I) according to the invention. For any one of these substituents, any of the definitions given below may be combined with any definition of any other substituent given below or elsewhere in this document.

Preferably T is 1 or 2, more preferably 1.

Preferably, R¹ is selected from the group consisting of hydrogen, halogen, C₁-C₆alkyl, C₁-C₆fluoroalkyl, —OR⁷ and —N(R^{7a})₂. More preferably, R¹ is selected from the group consisting of hydrogen, C₁-C₆alkyl, —OR⁷ and —N(R⁷)₂. Even more preferably, R¹ is hydrogen or C₁-C₆alkyl. Even preferably still, R¹ is hydrogen or methyl. Most preferably R¹ is hydrogen.

Preferably R² is hydrogen or C₁-C₆alkyl. More preferably, R² is hydrogen or methyl. Most preferably R² is hydrogen.

When R¹ and R² together with the carbon atom to which they are attached form a C₃-C₆cycloalkyl ring or a 3- to 6-membered heterocyclyl, then preferably, R¹ and R² together with the carbon atom to which they are attached form a cyclopropyl ring.

In one embodiment R¹ and R² are hydrogen.

Y is (CR^{1a}R^{2b})ₘ.

m is 1, 2 or 3. Preferably, m is 1 or 2. More preferably, m is or 1.

Preferably R^{1a} is selected from the group consisting of hydrogen, halogen, C₁-C₆alkyl, C₂-C₆alkenyl, C₂-C₆alkynyl, C₃-C₆cycloalkyl, C₁-C₆haloalkyl, —OH, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —NH₂, —NHR⁷, —N(R^{7a})₂ and —S(O)ᵣR¹⁵ and one of the following;

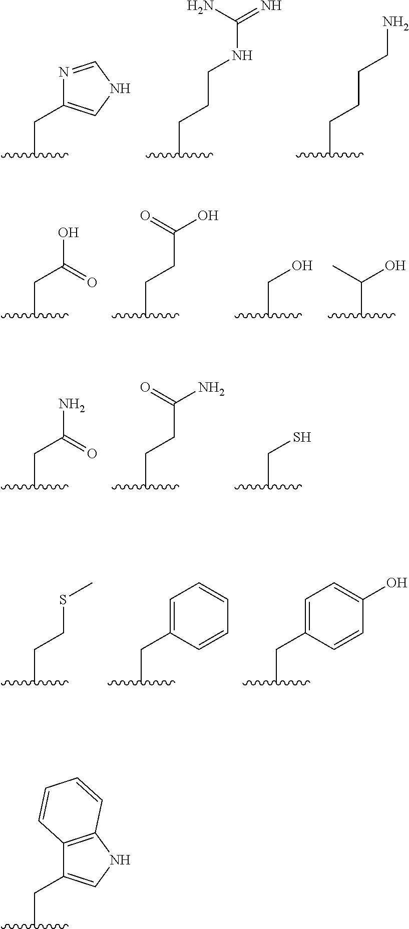

More preferably each R^{1a} is selected from the group consisting of hydrogen, halogen, C₁-C₆alkyl, C₂-C₆alkenyl, C₂-C₆alkynyl, C₃-C₆cycloalkyl, C₁-C₆haloalkyl, —OH, —OR⁷, —OR¹⁵, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —NH₂, —NHR⁷, —N(R^{7a})₂ and —S(O)ᵣR¹⁵. Even more preferably R^{1a} is selected from the group consisting of hydrogen, halogen, C₁-C₆alkyl, C₁-C₆fluoroalkyl, —OH, —NH₂ and —NHR⁷. More preferably still, R^{1a} is selected from the group consisting of hydrogen, C₁-C₆alkyl, —OH and —NH₂. Even more preferably stII, R^{1a} is selected from the group consisting of hydrogen and C₁-C₆alkyl, particularly hydrogen and methyl. Most preferably Ria is hydrogen.

Preferably R^{2b} is selected from the group consisting of hydrogen, halogen, C₁-C₆alkyl and C₁-C₆haloalkyl and one of the following;

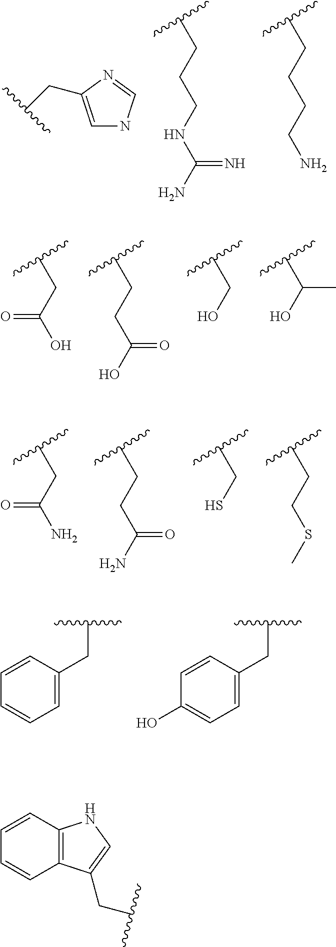

More preferably each R^{2b} are independently selected from the group consisting of hydrogen, halogen, C₁-C₆alkyl and C₁-C₆fluoroalkyl. Even more preferably each R^{2b} are independently selected from the group consisting of hydrogen and C₁-C₆alkyl. Still more preferably, R^{2b} is independently selected from the group consisting of hydrogen and methyl. Most preferably R^{2b} is hydrogen.

Alternatively, each R^{1a} and R^{2b} together with the carbon atom to which they are attached form a C₃-C₆cycloalkyl ring. Preferably in this case, each R^{1a} and R^{2b} together with the carbon atom to which they are attached form a cyclopropyl ring.

Preferably when R^{1a} is selected from the group consisting of —OH, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —NH₂, —NHR⁷, —NHR^{15a}, —N(R⁷)₂, —N(R^{7a})₂, —NR^{7b}R^{7c} and —S(O)ᵣR¹⁵, then the R^{2b} attached to the same carbon atom is selected from the group consisting of hydrogen and C₁-C₆alkyl.

Preferably, R³, R⁴ and R⁵ are independently selected from the group consisting of hydrogen, halogen, cyano, C₁-C₆alkyl, C₁-C₆fluoroalkyl, C₁-C₆fluoroalkoxy, C₁-C₆alkoxy, C₃-C₆cycloalkyl and —N(R⁶)₂. More preferably, R³, R⁴ and R⁵ are independently selected from the group consisting of hydrogen, halogen, cyano, C₁-C₆alkyl and C₁-C₆fluoroalkyl. Even more preferably, R³, R⁴ and R⁵ are independently selected from the group consisting of hydrogen, and C₁-C₃ alkyl. Even more preferably still, R³, R⁴ and R⁵ are independently selected from the group consisting of hydrogen and methyl. Most preferably, R³, R⁴ and R⁵ are hydrogen.

Preferably, each R⁶ is independently selected from hydrogen and methyl.

Preferably, each R⁷ is independently selected from the group consisting of C₁-C₆alkyl, —C(O)R¹⁵ and —C(O)NR¹⁶R¹⁷. More preferably, each R⁷ is C₁-C₆alkyl. Most preferably, each R⁷ is methyl.

Preferably, each R^{7a} is independently —C(O)R¹⁵ or —C(O)NR¹⁶R¹⁷.

Preferably, R^{7b} and R^{7c} are independently selected from the group consisting of C₁-C₆alkyl, —C(O)R¹⁵ and —C(O)NR¹⁶R¹⁷. More preferably, R^{7b} and R^{7c} are C₁-C₆alkyl. Most preferably, R^{7b} and R^{7c} are methyl.

A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different.

Preferably, A is a heteroaryl selected from the group consisting of 1,2,4-oxadiazol-5-yl, thiadiazol-5-yl, 1,2,4-thiadiazol-5-yl, thiadiazol-4-yl, 1,2,4-thiadiazol-3-yl, 1,2,5-thiadiazol-3-yl, 1,3,4-thiadiazol-2-yl, 1,3,4-oxadiazol-2-yl, 1,2,4-oxadiazol-3-yl, 1,2,5-oxadiazol-3-yl, 1,2,4-triazol-3-yl, 1,2,4-triazol-5-yl, triazol-4-yl, triazol-5-yl, 2-methyltetrazol-5-yl, 1-methyltetrazol-5-yl, thiazol-2-yl, thiazol-4-yl, isothiazol-5-yl, isothiazol-4-yl, isothiazol-3-yl, oxazol-2-yl, oxazol-4-yl, isoxazol-3-yl, isoxazol-5-yl, imidazol-5-yl, imidazol-2-yl, 3-furyl, 2-furyl, 3-thienyl, pyrazol-5-yl, pyrazol-3-yl and 2-thienyl wherein the heteroaryl may, where feasible, be optionally substituted by 1, 2 or 3 R⁸ substituents, which may be the same or different.

More preferably, A is selected from the group consisting of formula A-I to A-XXXV below

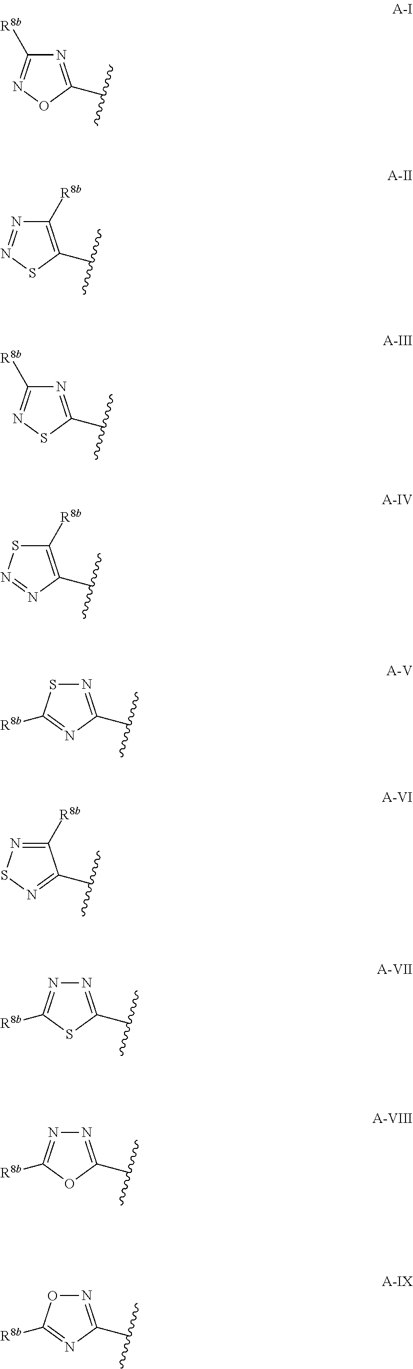

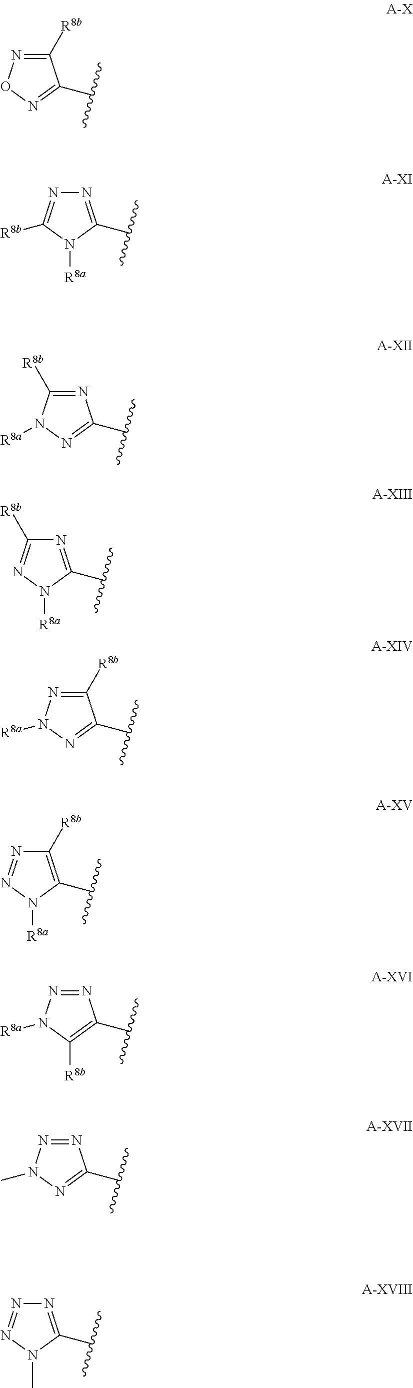

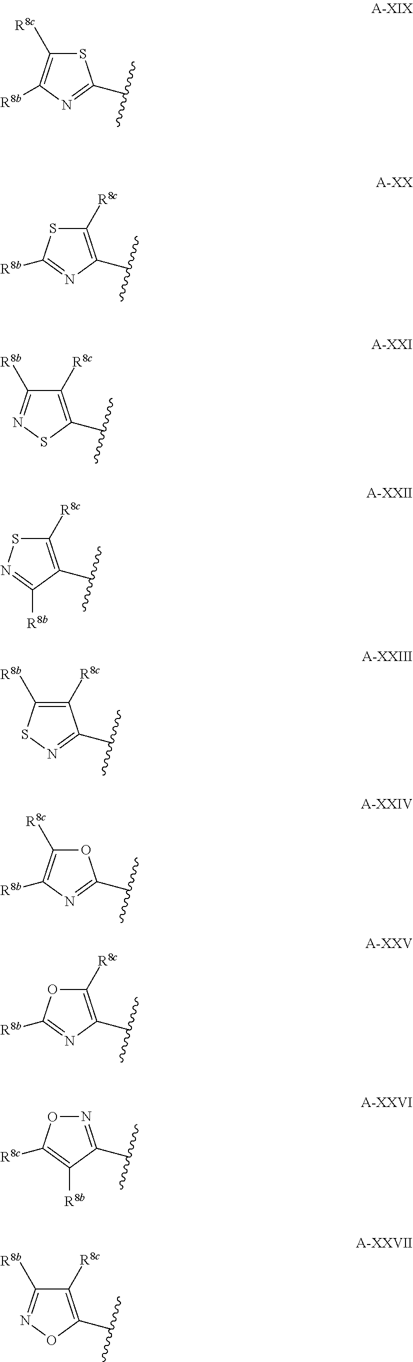

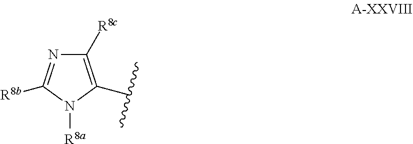

wherein the jagged line defines the point of attachment to a compound of formula (I), and R^{8a}, R^{8b}, R^{8c}, R^{8d}, R¹⁰, R¹⁵, R¹⁶, R¹⁷ and r are as defined herein. R^{8a}, R^{8b}, R^{8c}, R^{8d} are examples of R⁸ wherein the subscript letter a, b, c and d are used to denote positions within individual heterocycles (A-I to A-XXXIV).

Even more preferably, A is selected from the group consisting of formula A-I to A-XXXII below

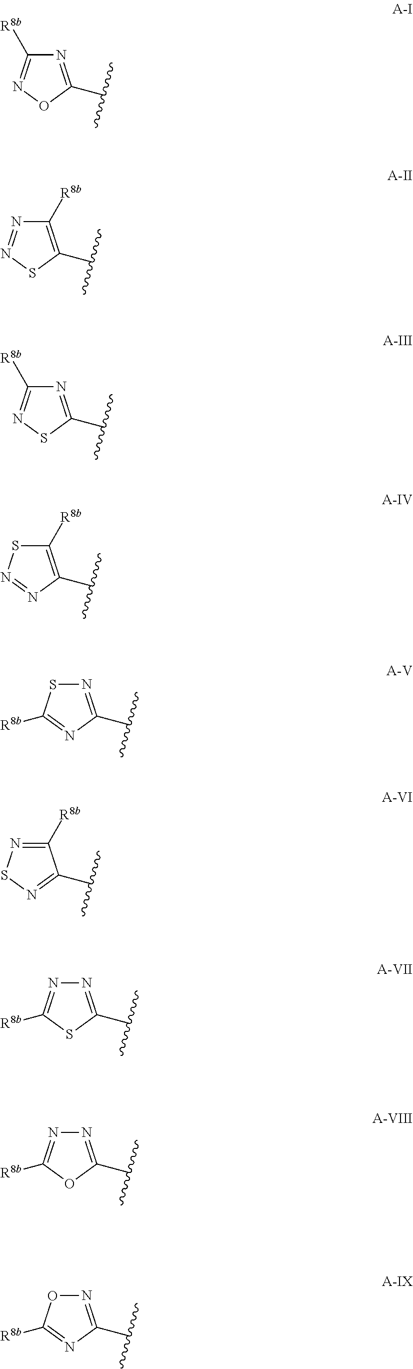

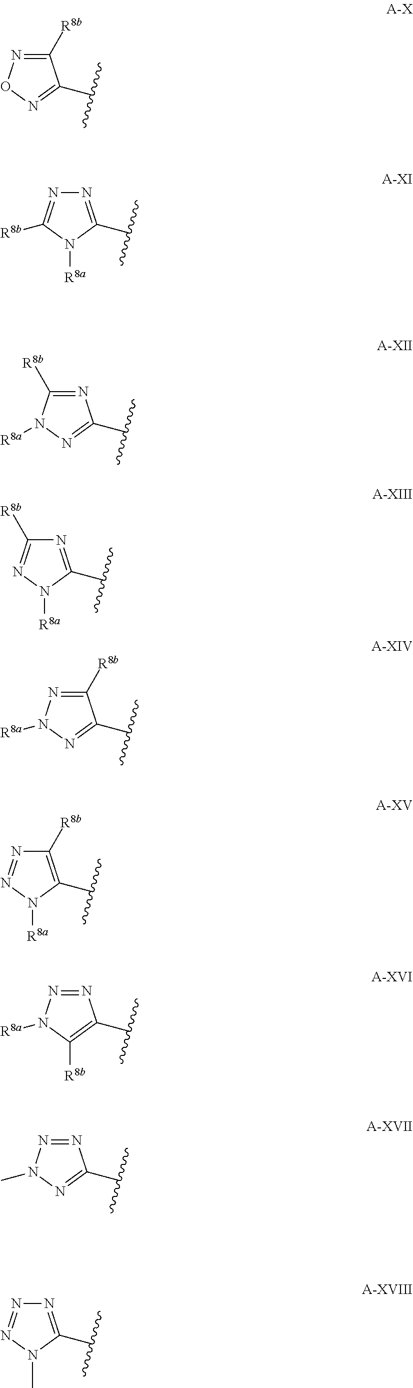

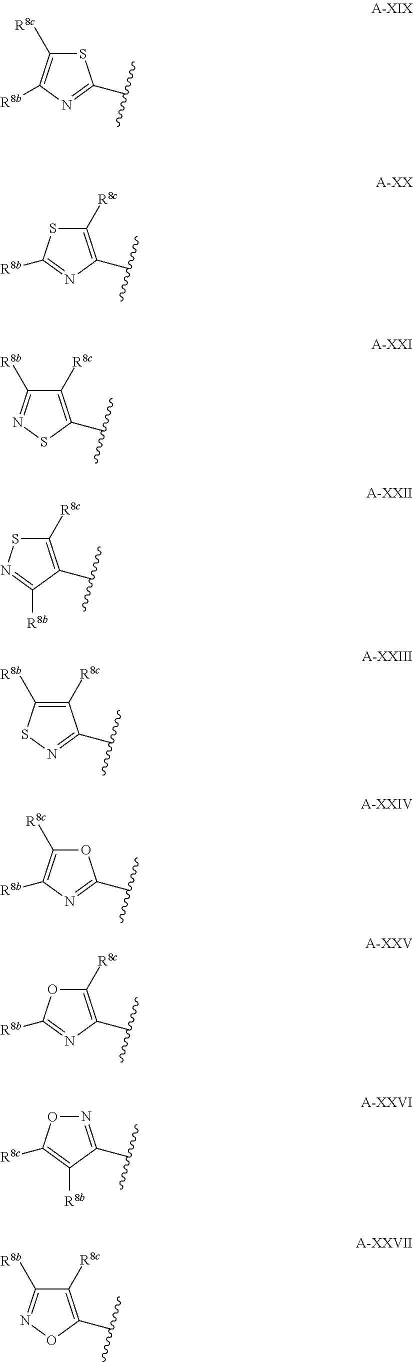

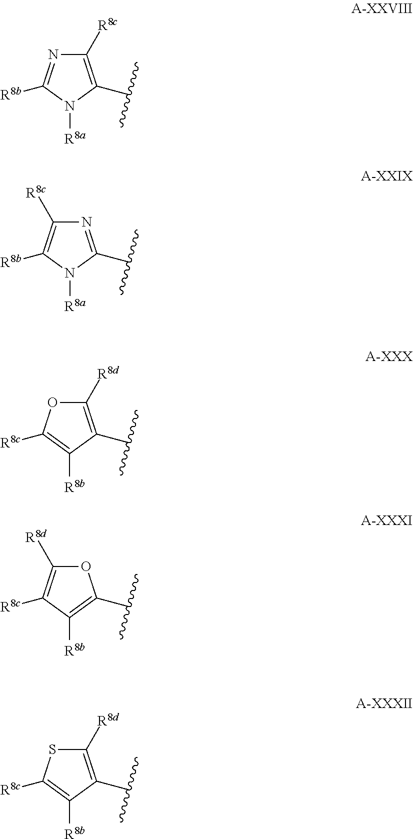

wherein the jagged line defines the point of attachment to a compound of formula (I), and R^{8a}, R^{8b}, R^{8c}, R^{8d}, R¹⁰, R¹⁵, R¹⁶, R¹⁷ and r are as defined herein.

Even more preferably still, A is selected from the group consisting of formula A-I to A-X, A-XVII, A-XVIII, A-XIX, A-XXIII, A-XXIV and AXXVII below:

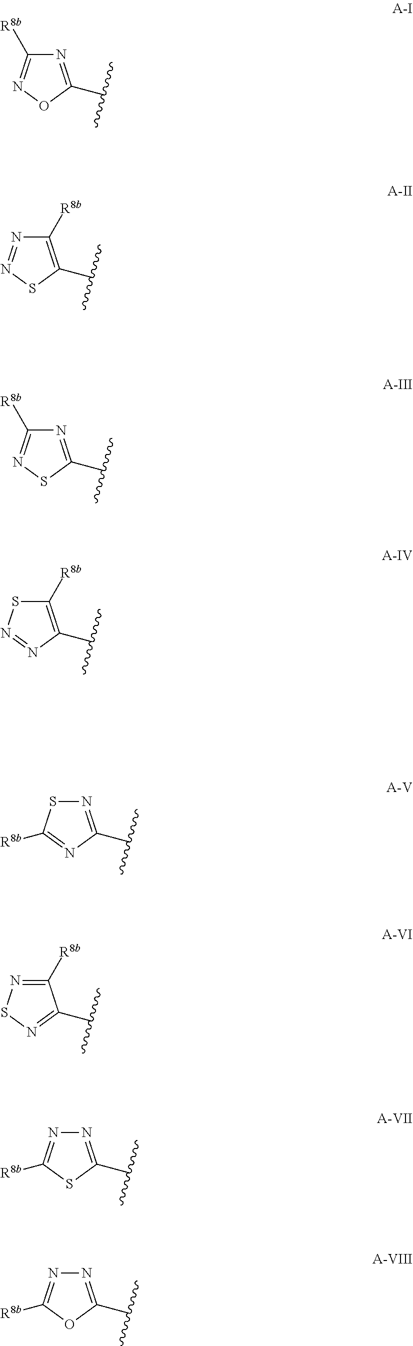

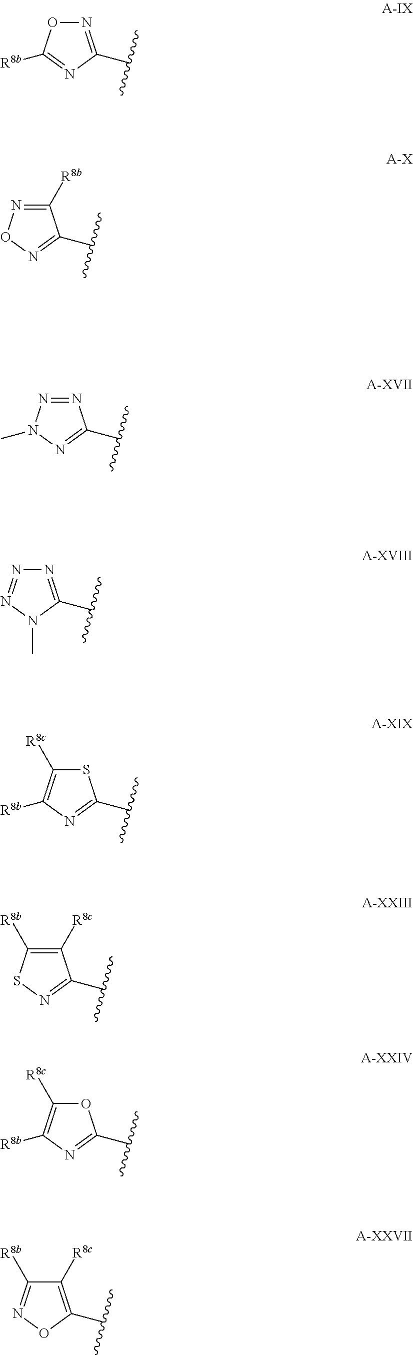

wherein the jagged line defines the point of attachment to a compound of formula (I), and R^{8a}, R^{8b}, R^{8c}, R^{8d}, R¹⁰, R¹⁵, R¹⁶, R¹⁷ and r are as defined herein.

Yet even more preferably still, A is selected from the group consisting of formula A-I to A-III below

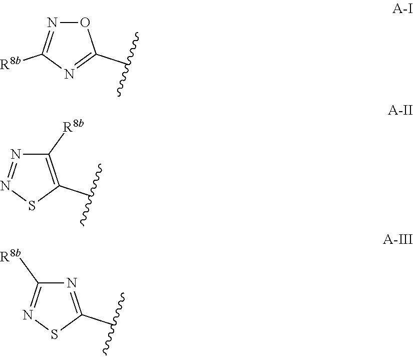

wherein the jagged line defines the point of attachment to a compound of formula (I), and each R^{8b} and R¹⁶ and R¹⁷ are as defined herein.

Further more preferably still, A is selected from the group consisting of formula A-Ia to A-VIIIa below:

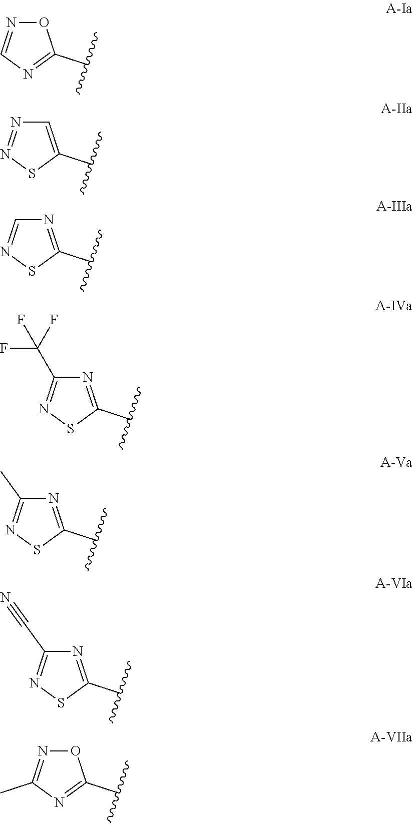

In one embodiment, A is selected from the group consisting of formula A-Ia to A-XXXIIIa below:

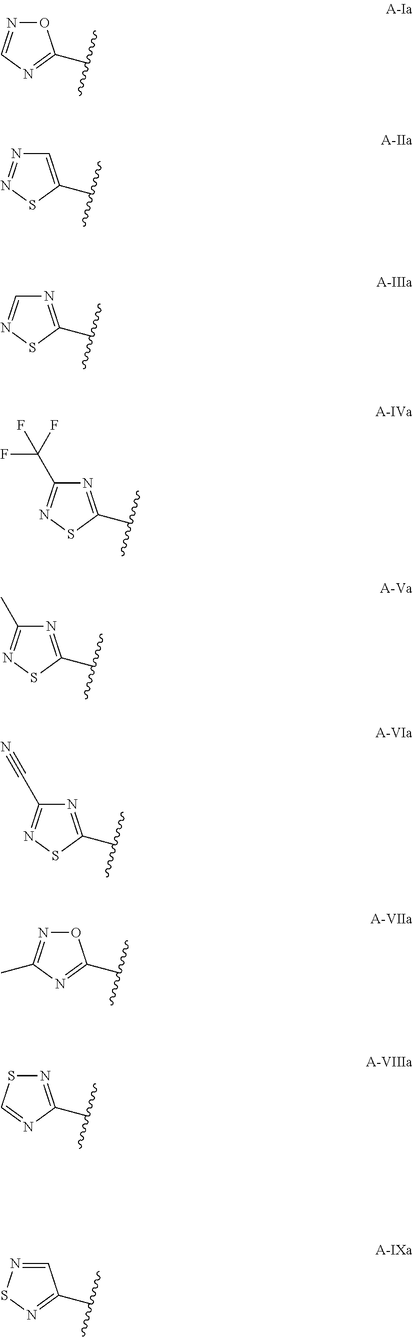

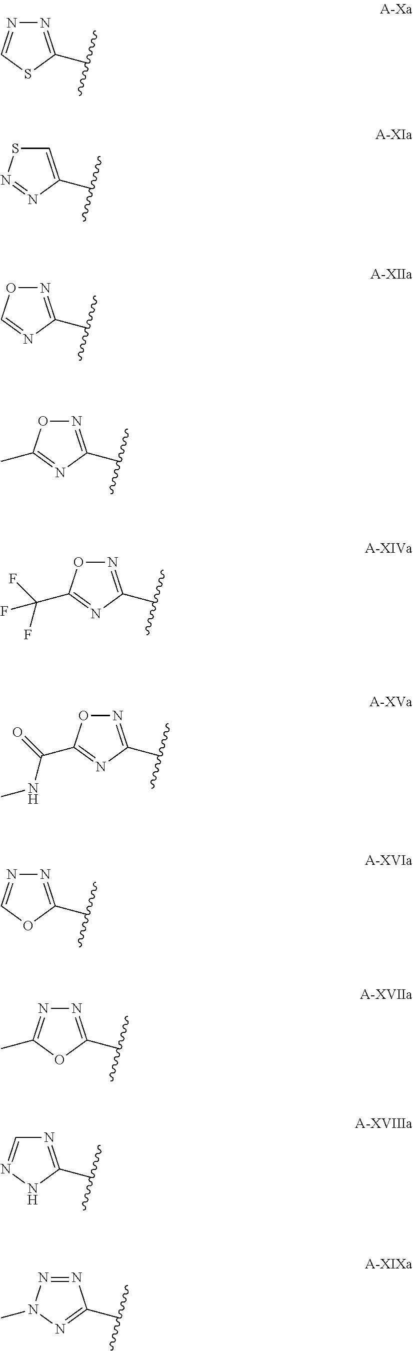

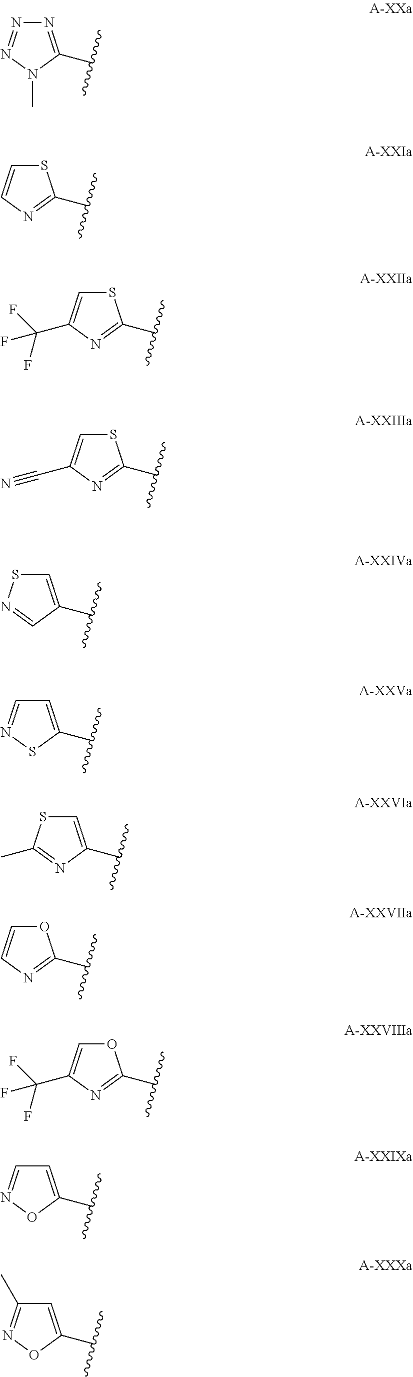

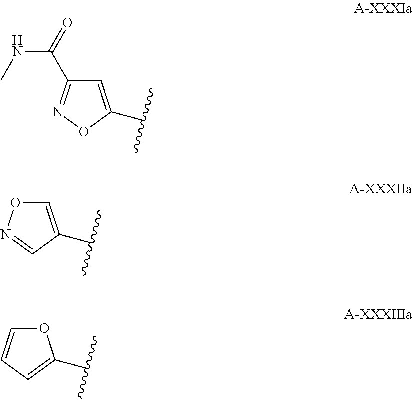

When A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different.

Preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl and a 5- or 6-membered heteroaryl, which comprises 1, 2, 3 or 4 heteroatoms individually selected from N, O and S, and wherein said phenyl or heteroaryl are optionally substituted by 1 or 2 R⁹ substituents, which may be the same or different.

More preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₁-C₃alkoxyC₁-C₃alkyl-, hydroxyC₁-C₆alkyl-, C₁-C₃alkoxyC₁-C₃alkoxy- and C₁-C₆haloalkoxy.

Even more preferably, when A is substituted on one or more ring carbon atoms, each R⁸ is independently selected from the group consisting of halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl.

Even more preferably still, when A is substituted on one or more ring carbon atoms, each R⁸ is independently selected from the group consisting of halogen, cyano, —NH₂, —C(O)NR¹⁶R¹⁷, C₁-C₆alkyl and C₁-C₆haloalkyl.

Further more preferably still, each R⁸ is independently selected from the group consisting of chloro, fluoro, cyano, —NH₂, —C(O)NH₂, —C(O)NHMe, —C(O)N(Me)₂, methyl and trifluoromethyl.

Most preferably, when A is substituted on one or more ring carbon atoms, each R⁸ is independently selected from the group consisting of —C(O)NHMe, methyl and trifluoromethyl.

When A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy. Preferably, R⁸ is selected from the group consisting of —OR, C₁-C₆alkyl and C₁-C₆haloalkyl. More preferably, each R⁸ is C₁-C₆alky or C₁-C₆haloalkyl. Even more preferably still, R⁸ is C₁-C₆alky. Most preferably R⁸ is methyl.

When A is selected from the group consisting of formula A-I to A-XXXIV, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably, R^{8a} is hydrogen or C₁-C₆alkyl and each R^{8b}, R^{8c} and R^{8d} are independently selected from the group consisting of hydrogen, halogen, cyano, —NH₂, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, chloro, fluoro, cyano, —NH₂, —C(O)NH₂, —C(O)NHMe, —C(O)N(Me)₂, methyl and trifluoromethyl. Even more preferably, R^{8a} is hydrogen or methyl and each R^{8b}, R^{8c} and R^{8d} are independently selected from the group consisting of hydrogen, —C(O)NHMe, methyl and trifluoromethyl.

When A is selected from the group consisting of formula A-I to A-XXXII, R^{8a} (substituted on a ring nitrogen atom) is selected from the group consisting of hydrogen, C₁-C₆alkyl and C₁-C₆haloalkyl, and

each R^{8b}, R^{8c} and R^{8d} (substituted on a ring carbon atom) are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably, R^{8a} is hydrogen or C₁-C₆alkyl and each R^{8b}, R^{8c} and R^{8d} are independently selected from the group consisting of hydrogen, halogen, cyano, —NH₂, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, chloro, fluoro, cyano, —NH₂, —C(O)NH₂, —C(O)NHMe, —C(O)N(Me)₂, methyl and trifluoromethyl. Even more preferably, R^{8a} is hydrogen or methyl and each R^{8b}, R^{8c} and R^{8d} are independently selected from the group consisting of hydrogen, —C(O)NHMe, methyl and trifluoromethyl.

When A is selected from the group consisting of formula A-I to A-X, A-XVII, A-XVIII, A-XIX, A-XXIII, A-XXIV and AXXVII, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably, R^{8a} is hydrogen or C₁-C₆alkyl and each R^{8b}, R^{8c} and R^{8d} are independently selected from the group consisting of hydrogen, halogen, cyano, —NH₂, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, chloro, fluoro, cyano, —NH₂, —C(O)NH₂, —C(O)NHMe, —C(O)N(Me)₂, methyl and trifluoromethyl. Even more preferably, R^{8a} is hydrogen or methyl and each R^{8b}, R^{8c} and R^{8d} are independently selected from the group consisting of hydrogen, —C(O)NHMe, methyl and trifluoromethyl.

When A is selected from the group consisting of formula A-I to A-III, each R^{8b} (substituted on a ring carbon atom) is independently selected from the group consisting of hydrogen, halogen, cyano, —NH₂, —C(O)NR¹⁶R¹⁷, C₁-C₆alkyl and C₁-C₆haloalkyl. Preferably, each R^{8b} is independently selected from the group consisting of hydrogen, chloro, fluoro, cyano, —NH₂, —C(O)NH₂, —C(O)NHMe, —C(O)N(Me)₂, methyl and trifluoromethyl. More preferably, each R^{8b} is independently selected from the group consisting of hydrogen, —C(O)NHMe, methyl and trifluoromethyl.

Each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy. Preferably, each R⁹ is independently selected from the group consisting of halogen, C₁-C₄alkyl, C₁-C₄alkoxy and C₁-C₄haloalkyl. More preferably, each R⁹ is independently selected from the group consisting of halogen and C₁-C₄alkyl.

The moieties from which X is selected can be represented for clarity by the structural formulas given in the following table;

| No. | X Structure |
|---|---|
| 1 | 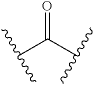 [SMILES: *C(*)=O] |
| 2 | 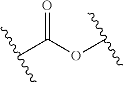 [SMILES: *OC(*)=O] |
| 3 | 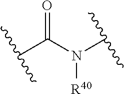 |
| 4 | 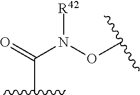 |
| 5 | 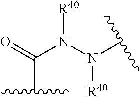 |
| 6 | 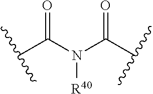 |
| 7 | 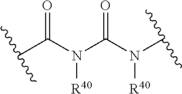 |
| 8 | 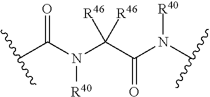 |
| 9 | 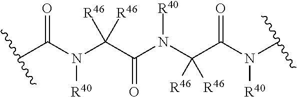 |
| 10 | 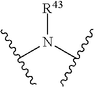 |
| 11 | 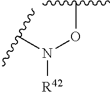 |
| 12 | 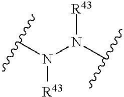 |
| 13 | 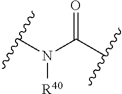 |
| 14 | 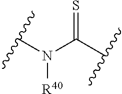 |
| 15 | 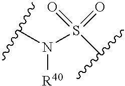 |
| 16 | 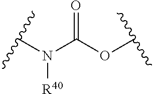 |
| 17 | 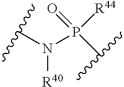 |
| 18 | 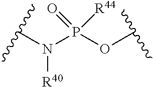 |
| 19 | 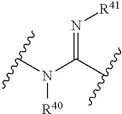 |
| 20 | 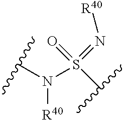 |
| 21 | 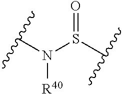 |
| 22 | 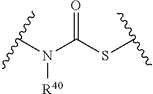 |
| 23 | 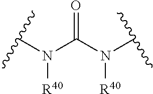 |
| 24 | 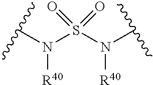 |
| 25 | 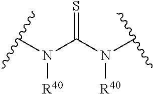 |
| 26 | 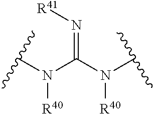 |
| 27 | 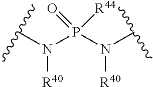 |
| 28 | 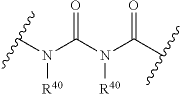 |
| 29 | 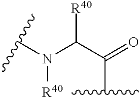 |
| 30 | 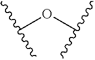 [SMILES: *O*] |
| 31 | 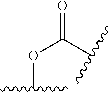 [SMILES: *OC(*)=O] |
| 32 | 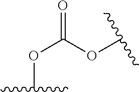 [SMILES: *OC(=O)O*] |
| 33 | 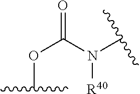 |
| 34 | 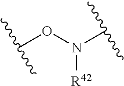 |
| 35 | 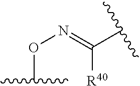 |
| 36 | 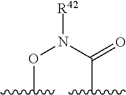 |
| 37 | 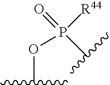 |
| 38 | 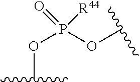 |
| 39 | 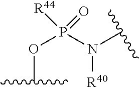 |
| 40 | 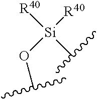 |
| 41 | 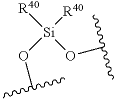 |
| 42 | 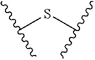 [SMILES: *S*] |
| 43 | 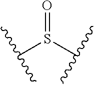 [SMILES: *S(*)=O] |
| 44 | 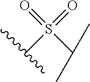 [SMILES: *S(=O)(=O)C(C)C] |
| 45 | 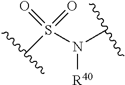 |
| 46 | 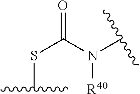 |
| 47 | 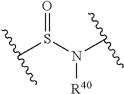 |
| 48 | 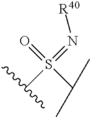 |
| 49 | 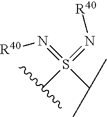 |
| 50 | 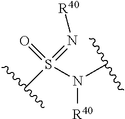 |
| 51 | 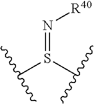 |
| 52 | 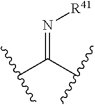 |
| 53 | 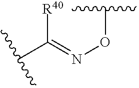 |
| 54 | 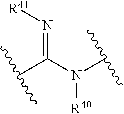 |
| 55 | 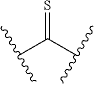 [SMILES: *C(*)=S] |
| 56 | 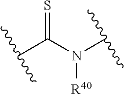 |
| 57 | 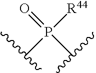 |
| 58 | 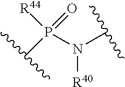 |
| 59 | 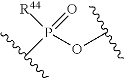 |
| 60 | 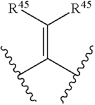 |
| 61 | 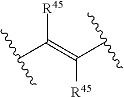 |
| 62 | 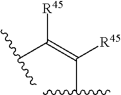 |
| 63 | 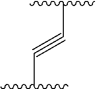 [SMILES: *C#C*] |
| 64 | 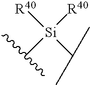 |
| 65 | 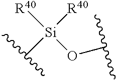 |

Preferably X is independently selected from the group consisting of —C(O)—, —C(O)N(R⁴⁰)—, —O—, —S(O)—, —S(O)₂—, —S(O)₂N(R⁴⁰)—, —N(R⁴⁰)C(O)—, —N(R⁴⁰)S(O)₂—, and —N(R⁴⁰)C(O)N(R⁴⁰)—

More preferably X is independently selected from the group consisting of —C(O)—, —C(O)N(R⁴⁰)—, —S(O)—, —S(O)₂— and —S(O)₂N(R⁴⁰)—, even more preferably —C(O)N(R⁴⁰)—, —S(O)—, —S(O)₂— and —S(O)₂N(R⁴⁰)— and most preferably X is —C(O)N(R⁴⁰)—

Preferably R⁴⁰ is selected from the group consisting of hydrogen and C₁-C₆alkyl, more preferably hydrogen or methyl;

Preferably R⁴¹ is selected from the group consisting of hydrogen and C₁-C₆alkyl, more preferably hydrogen and methyl;

Preferably R⁴² is selected from the group consisting of hydrogen and C₁-C₆alkyl, more preferably hydrogen and methyl;

Preferably R⁴³ is selected from the group consisting of hydrogen and C₁-C₆alkyl, more preferably hydrogen and methyl;

Preferably R⁴⁴ is selected from the group consisting of C₁-C₆alkyl and C₁-C₆alkoxy, more preferably methyl and methoxy;

Preferably R⁴⁵ is selected from the group consisting of hydrogen and C₁-C₆alkyl more preferably hydrogen and methyl. Preferably R⁴⁶ is selected from the group consisting of hydrogen, C₁-C₆alkyl, C₁-C₆alkoxy, C₁-C₆alkoxyC₁-C₃alkyl, and one of the following:

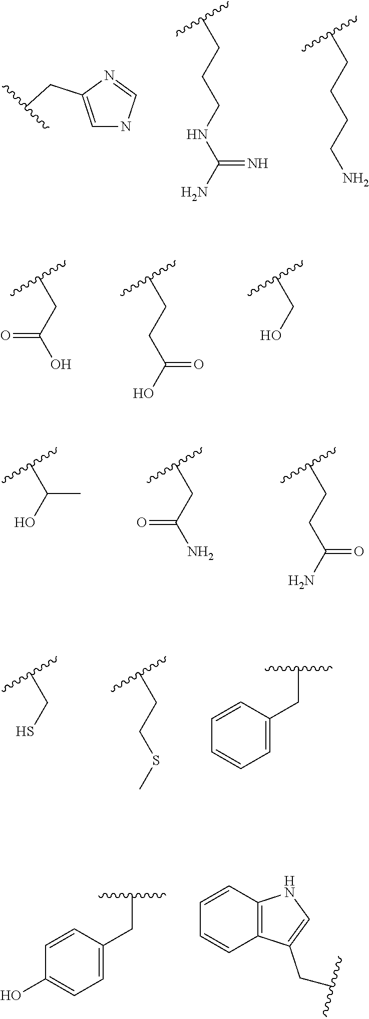

In one embodiment where X is —C(O)— then Y—Z is a peptide moiety comprising one or two amino acid moieties independently selected from the group consisting of Ala, Cys, Asp, Glu, Phe, Gly, His, lie, Lys, Leu, Met, Asn, Pro, Gln, Arg, Ser, Thr, Val, Trp and Tyr, wherein said peptide moiety is bonded to the rest of the molecule via a nitrogen atom in the amino acid moiety;

More preferably R⁴⁶ is selected from the group consisting of hydrogen and C₁-C₆alkyl, most preferably hydrogen and methyl.

Z is selected from the group consisting of —C(O)OR¹⁰, —OH, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole.

Preferably, Z is selected from the group consisting of —C(O)OR¹⁰, —C(O)NHOR¹¹, —OC(O)NHOR¹¹, —NR⁶C(O)NHOR¹¹, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHC(O)R¹⁸, —S(O)₂NHS(O)₂R¹², —OS(O)₂NHS(O)₂R¹², —OS(O)₂NHC(O)R¹⁸, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰) and —NR⁶P(O)(R¹³)(OR¹⁰).

More preferably, Z is selected from the group consisting of —C(O)OR¹⁰, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰ and —P(O)(R¹³)(OR¹⁰).

Even more preferably Z is selected from the group consisting of —C(O)OR¹⁰, —C(O)NHS(O)₂R¹², —S(O)₂OR¹⁰, and —P(O)(R¹³)(OR¹⁰).

Even more preferably still Z is selected from the group consisting of —C(O)OH, —C(O)OCH₃, —C(O)OCH₂CH₃, —C(O)OCH(CH₃)₂, —C(O)OC(CH₃)₃, —C(O)OCH₂C₆H₅, —C(O)OC₆H₅, —C(O)NHS(O)₂CH₃, —S(O)₂OH, —P(O)(OH)(OCH₂CH₃) and —P(O)(OCH₂CH₃)(OCH₂CH₃).

Most preferably Z is —C(O)OH or —S(O)₂OH.

Preferably, R¹⁰ is selected from the group consisting of hydrogen, C₁-C₆alkyl, phenyl and benzyl. More preferably, R¹⁰ is selected from the group consisting of hydrogen and C₁-C₆alkyl. Most preferably, R¹⁰ is hydrogen.

Preferably, R¹¹ is selected from the group consisting of hydrogen, C₁-C₆alkyl and phenyl. More preferably, R¹¹ is selected from the group consisting of hydrogen and C₁-C₆alkyl. Even more preferably, R¹¹ is C₁-C₆alkyl. Most preferably, R¹¹ is methyl.

Preferably, R¹² is selected from the group consisting of C₁-C₆alkyl, C₁-C₆haloalkyl, C₁-C₆alkoxy, —OH, —N(R⁶)₂ and phenyl. More preferably, R¹² is selected from the group consisting of C₁-C₆alkyl, C₁-C₆haloalkyl and —N(R⁶)₂. Even more preferably, R¹² is selected from the group consisting of methyl, —N(CH₃)₂ and trifluoromethyl. Most preferably, R¹² is methyl.

Preferably R¹³ is selected from the group consisting of —OH, C₁-C₆alkyl and C₁-C₆alkoxy. More preferably, R¹³ is selected from the group consisting of —OH and C₁-C₆alkoxy. Even more preferably, R¹³ is selected from the group consisting of —OH, methoxy and ethoxy. Most preferably, R¹³ is —OH.

Preferably, R¹⁴ is trifluoromethyl.

Preferably, R¹⁵ is selected from the group consisting of C₁-C₆alkyl and phenyl. More preferably, R¹⁵ is C₁-C₆alkyl. Most preferably R¹⁵ is methyl.

Preferably, R¹⁶ and R¹⁷ are independently selected from the group consisting of hydrogen and methyl, or R¹⁶ and R¹⁷ together with the nitrogen atom to which they are attached form a 5- to 6-membered heterocyclyl ring which optionally comprises one additional heteroatom individually selected from N and O. More preferably, R¹⁶ and R¹⁷ together with the nitrogen atom to which they are attached form an pyrrolidyl, oxazolidinyl, imidazolidinyl, piperidyl, piperazinyl or morpholinyl group.

Preferably, R¹⁸ is selected from the group consisting of hydrogen, C₁-C₆alkyl, C₁-C₆haloalkyl, C₁-C₆alkoxy, —N(R⁶)₂ and phenyl. More preferably, R¹⁸ is selected from the group consisting of hydrogen, C₁-C₆alkyl and C₁-C₆haloalkyl. Further more preferably, R¹⁸ is selected from the group consisting of C₁-C₆alkyl and C₁-C₆haloalkyl. Most preferably, R¹⁸ is methyl or trifluoromethyl.

Preferably, r is 0 or 2.

In a set of preferred embodiments, in a compound according to formula (I) of the invention,
R¹ is hydrogen or C₁-C₆alkyl;
R² is hydrogen or methyl;
Y is (CR^{1a}R^{2b})ₘ;
m is 1 or 2;
R^{1a} and R^{2b} are independently selected from the group consisting of hydrogen and C₁-C₆alkyl;
R³, R⁴ and R⁵ are independently selected from the group consisting of hydrogen and C₁-C₆alkyl;
each R⁶ is independently selected from hydrogen and methyl;
each R⁷ is C₁-C₆alkyl;
A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different;
when A is substituted on one or more ring carbon atoms, each R⁸ is independently selected from the group consisting of halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl;
and/or
when A is substituted on a ring nitrogen atom, R⁸ is C₁-C₆alky or C₁-C₆haloalkyl; and
n is 0;
Z is selected from the group consisting of —C(O)OR¹⁰, —C(O)NHS(O)₂R¹², —S(O)₂OR¹⁰, and —P(O)(R¹³)(OR¹⁰);
R¹⁰ is selected from the group consisting of hydrogen, C₁-C₆alkyl, phenyl and benzyl;
R¹² is selected from the group consisting of C₁-C₆alkyl, C₁-C₆haloalkyl and —N(R⁶)₂;
R¹³ is selected from the group consisting of —OH and C₁-C₆alkoxy;
R¹⁵ is C₁-C₆alkyl;
R¹⁶ and R¹⁷ are independently selected from the group consisting of hydrogen and methyl; and
r is 0 or 2.

More preferably,
R¹ is hydrogen or methyl;
R² is hydrogen or methyl;
Y is (CR^{1a}R^{2b})ₘ.
m is 1 or 2;
R^{1a} and R^{2b} are independently selected from the group consisting of hydrogen and methyl;
R³, R⁴ and R⁵ are independently selected from the group consisting of hydrogen and methyl;
A is a heteroaryl selected from the group consisting of 1,2,4-oxadiazol-5-yl, thiadiazol-5-yl, 1,2,4-thiadiazol-5-yl, thiadiazol-4-yl, 1,2,4-thiadiazol-3-yl, 1,2,5-thiadiazol-3-yl, 1,3,4-thiadiazol-2-yl, 1,3,4-oxadiazol-2-yl, 1,2,4-oxadiazol-3-yl, 1,2,5-oxadiazol-3-yl, 1,2,4-triazol-3-yl, 1,2,4-triazol-5-yl, triazol-4-yl, triazol-5-yl, 2-methyltetrazol-5-yl, 1-methyltetrazol-5-yl, thiazol-2-yl, thiazol-4-yl, isothiazol-5-yl, isothiazol-4-yl, isothiazol-3-yl, oxazol-2-yl, oxazol-4-yl, isoxazol-3-yl, isoxazol-5-yl, imidazol-5-yl, imidazol-2-yl, 3-furyl, 2-furyl, 3-thienyl, pyrazol-5-yl, pyrazol-3-yl and 2-thienyl wherein the heteroaryl may, where feasible, be optionally substituted by 1, 2 or 3 R⁸ substituents, which may be the same or different;
when A is substituted on one or more ring carbon atoms, each R⁸ is independently selected from the group consisting of halogen, cyano, —NH₂, —C(O)NR¹⁶R¹⁷, C₁-C₆alkyl and C₁-C₆haloalkyl;
and/or
when A is substituted on a ring nitrogen atom, R⁸ is C₁-C₆alkyl; and
n is 0; and
and
Z is selected from the group consisting of —C(O)OH, —C(O)OCH₃, —C(O)OCH₂CH₃, —C(O)OCH(CH₃)₂, —C(O)OC(CH₃)₃, —C(O)OCH₂C₆H₅, —C(O)OC₆H₅, —C(O)NHS(O)₂CH₃, —S(O)₂OH, —P(O)(OH)(OCH₂CH₃) and —P(O)(OCH₂CH₃)(OCH₂CH₃).

In one set of embodiments, the compound according to formula (I) is selected from a compound A1 to A12 listed in Table A.

It should be understood that compounds of formula (I) may exist/be manufactured in ‘procidal form’, wherein they comprise a group ‘G’. Such compounds are referred to herein as compounds of formula (I-IV).

G is a group which may be removed in a plant by any appropriate mechanism including, but not limited to, metabolism and chemical degradation to give a compound of formula (I-I) or (I-II), wherein Z contains an acidic proton, see scheme below:

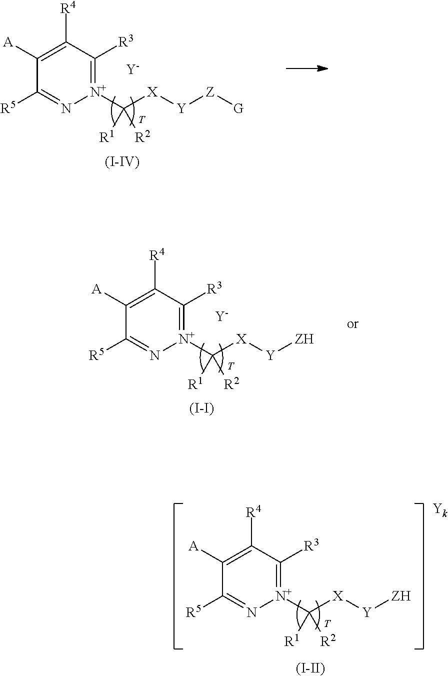

Whilst such G groups may be considered as ‘procidal’, and thus yield active herbicidal compounds once removed, compounds comprising such groups may also exhibit herbicidal activity in their own right. In such cases in a compound of formula (I-IV), Z-G may include but is not limited to, any one of (G1) to (G7) below and E indicates the point of attachment to a compound of formula (I):

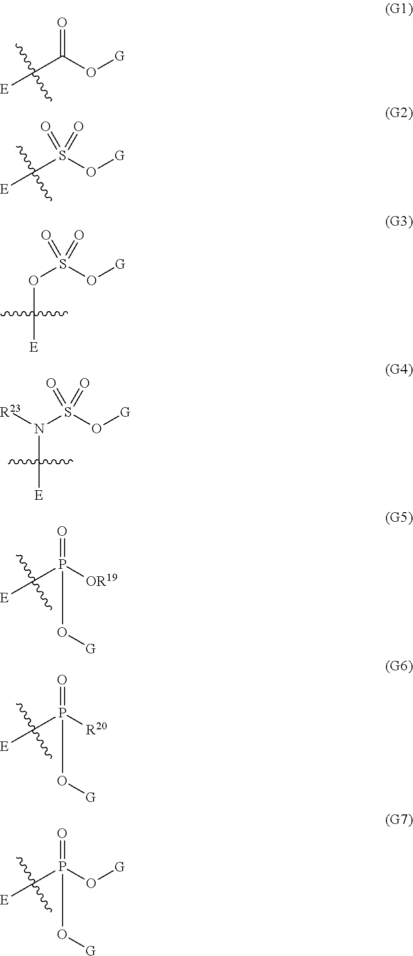

In embodiments where Z is (G1) to (G7), G, R¹⁹, R²⁰, R²¹, R²² and R²³ are defined herein: G is C₁-C₆alkyl, C₂-C₆alkenyl, C₂-C₆alkynyl, —C(R²¹R²²)OC(O)R¹⁹, phenyl or phenyl-C₁-C₄alkyl-, wherein said phenyl moiety is optionally substituted by 1 to 5 substituents independently selected from halo, cyano, nitro, C₁-C₆alkyl, C₁-C₆haloalkyl or C₁-C₆alkoxy.

R¹⁹ is C₁-C₆alkyl or phenyl,
R²⁰ is hydroxy, C₁-C₆alkyl, C₁-C₆alkoxy or phenyl,
R²¹ is hydrogen or methyl,
R²² is hydrogen or methyl,
R²³ is hydrogen or C₁-C₆alkyl.

The compounds in Tables 1 to 80 below illustrate the compounds of the invention. The skilled person would understand that the compounds of formula (I) may exist as an agronomically acceptable salt, a zwitterion or an agronomically acceptable salt of a zwitterion as described hereinbefore.

TABLE 1
This table discloses 78 specific compounds of the formula (T-1):

| | |
|---|---|
| | (T-1) |
| 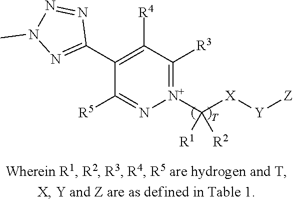 | |

| Com- | | | | |
|---|---|---|---|---|
| pound | | | | |
| num- | | | | |
| ber | X | Y | Z | T |
| 1.001 | —C(O)— | CH₂ | —C(O)OH | 1 |
| 1.002 | —C(O)— | CH₂ | —C(O)NHS(O)₂Me | 1 |
| 1.003 | —C(O)— | CH₂ | —S(O)₂OH | 1 |
| 1.004 | —C(O)— | CH₂ | —P(O)(OH)(OMe) | 1 |
| 1.005 | —C(O)— | CH₂ | —P(O)(OH)(Me) | 1 |
| 1.006 | —C(O)— | CH₂ | —P(O)(OH)(OH) | 1 |
| 1.007 | —NHC(O)— | CH₂ | —C(O)OH | 1 |
| 1.008 | —NHC(O)— | CH₂ | —C(O)NHS(O)₂Me | 1 |
| 1.009 | —NHC(O)— | CH₂ | —S(O)₂OH | 1 |
| 1.010 | —NHC(O)— | CH₂ | —P(O)(OH)(OMe) | 1 |
| 1.011 | —NHC(O)— | CH₂ | —P(O)(OH)(Me) | 1 |
| 1.012 | —NHC(O)— | CH₂ | —P(O)(OH)(OH) | 1 |
| 1.013 | —C(O)NH— | CH₂ | —C(O)OH | 1 |
| 1.014 | —C(O)NH | CH₂ | —C(O)NHS(O)₂Me | 1 |
| 1.015 | —C(O)NH | CH₂ | —S(O)₂OH | 1 |
| 1.016 | —C(O)NH | CH₂ | —P(O)(OH)(OMe) | 1 |
| 1.017 | —C(O)NH | CH₂ | —P(O)(OH)(Me) | 1 |
| 1.018 | —C(O)NH | CH₂ | —P(O)(OH)(OH) | 1 |
| 1.019 | —NHC(O)NH— | CH₂ | —C(O)OH | 1 |
| 1.020 | —NHC(O)NH— | CH₂ | —C(O)NHS(O)₂Me | 1 |
| 1.021 | —NHC(O)NH— | CH₂ | —S(O)₂OH | 1 |
| 1.022 | —NHC(O)NH— | CH₂ | —P(O)(OH)(OMe) | 1 |
| 1.023 | —NHC(O)NH— | CH₂ | —P(O)(OH)(Me) | 1 |
| 1.024 | —NHC(O)NH— | CH₂ | —P(O)(OH)(OH) | 1 |
| 1.025 | —O— | CH₂ | —C(O)OH | 1 |
| 1.026 | —O— | CH₂ | —C(O)NHS(O)₂Me | 1 |
| 1.027 | —O— | CH₂ | —S(O)₂OH | 1 |
| 1.028 | —O— | CH₂ | —P(O)(OH)(OMe) | 1 |
| 1.029 | —O— | CH₂ | —P(O)(OH)(Me) | 1 |
| 1.030 | —O— | CH₂ | —P(O)(OH)(OH) | 1 |
| 1.031 | —S— | CH₂ | —C(O)OH | 1 |
| 1.032 | —S— | CH₂ | —C(O)NHS(O)₂Me | 1 |
| 1.033 | —S— | CH₂ | —S(O)₂OH | 1 |
| 1.034 | —S— | CH₂ | —P(O)(OH)(OMe) | 1 |
| 1.035 | —S— | CH₂ | —P(O)(OH)(Me) | 1 |
| 1.036 | —S— | CH₂ | —P(O)(OH)(OH) | 1 |
| 1.037 | —SO— | CH₂ | —C(O)OH | 1 |
| 1.038 | —SO— | CH₂ | —C(O)NHS(O)₂Me | 1 |
| 1.039 | —SO— | CH₂ | —S(O)₂OH | 1 |
| 1.040 | —SO— | CH₂ | —P(O)(OH)(OMe) | 1 |
| 1.041 | —SO— | CH₂ | —P(O)(OH)(Me) | 1 |
| 1.042 | —SO— | CH₂ | —P(O)(OH)(OH) | 1 |
| 1.043 | —SO₂— | CH₂ | —C(O)OH | 1 |
| 1.044 | —SO₂— | CH₂ | —C(O)NHS(O)₂Me | 1 |
| 1.045 | —SO₂— | CH₂ | —S(O)₂OH | 1 |
| 1.046 | —SO₂— | CH₂ | —P(O)(OH)(OMe) | 1 |
| 1.047 | —SO₂— | CH₂ | —P(O)(OH)(Me) | 1 |
| 1.048 | —SO₂— | CH₂ | —P(O)(OH)(OH) | 1 |
| 1.049 | —NHS(O)₂— | CH₂ | —C(O)OH | 1 |
| 1.050 | —NHS(O)₂— | CH₂ | —C(O)NHS(O)₂Me | 1 |
| 1.051 | —NHS(O)₂— | CH₂ | —S(O)₂OH | 1 |
| 1.052 | —NHS(O)₂— | CH₂ | —P(O)(OH)(OMe) | 1 |
| 1.053 | —NHS(O)₂— | CH₂ | —P(O)(OH)(Me) | 1 |
| 1.054 | —NHS(O)₂— | CH₂ | —P(O)(OH)(OH) | 1 |
| 1.055 | —S(O)₂NH— | CH₂ | —C(O)OH | 1 |
| 1.056 | —S(O)₂NH— | CH₂ | —C(O)NHS(O)₂Me | 1 |
| 1.057 | —S(O)₂NH— | CH₂ | —S(O)₂OH | 1 |
| 1.058 | —S(O)₂NH— | CH₂ | —P(O)(OH)(OMe) | 1 |
| 1.059 | —S(O)₂NH— | CH₂ | —P(O)(OH)(Me) | 1 |
| 1.060 | —S(O)₂NH— | CH₂ | —P(O)(OH)(OH) | 1 |
| 1.061 | —C(O)NMe— | CH₂ | —C(O)OH | 1 |
| 1.062 | —C(O)NMe— | CH₂ | —C(O)NHS(O)₂Me | 1 |
| 1.063 | —C(O)NMe— | CH₂ | —S(O)₂OH | 1 |
| 1.064 | —C(O)NMe— | CH₂ | —P(O)(OH)(OMe) | 1 |
| 1.065 | —C(O)NMe— | CH₂ | —P(O)(OH)(Me) | 1 |
| 1.066 | —C(O)NMe— | CH₂ | —P(O)(OH)(OH) | 1 |
| 1.067 | —C(O)NH— | CHMe | —C(O)OH | 1 |
| 1.068 | —C(O)NH— | CHMe | —C(O)NHS(O)₂Me | 1 |
| 1.069 | —C(O)NH— | CHMe | —S(O)₂OH | 1 |
| 1.070 | —C(O)NH— | CHMe | —P(O)(OH)(OMe) | 1 |
| 1.071 | —C(O)NH— | CHMe | —P(O)(OH)(Me) | 1 |
| 1.072 | —C(O)NH— | CHMe | —P(O)(OH)(OH) | 1 |
| 1.073 | —C(O)NMe— | CHMe | —C(O)OH | 1 |
| 1.074 | —C(O)NMe— | CHMe | —C(O)NHS(O)₂Me | 1 |
| 1.075 | —C(O)NMe— | CHMe | —S(O)₂OH | 1 |
| 1.076 | —C(O)NMe— | CHMe | —P(O)(OH)(OMe) | 1 |
| 1.077 | —C(O)NMe— | CHMe | —P(O)(OH)(Me) | 1 |
| 1.078 | —C(O)NMe— | CHMe | —P(O)(OH)(OH) | 1 |

TABLE 2
This table discloses 60 specific compounds of the formula (T-2):

| | |
|---|---|
| | (T-2) |
| 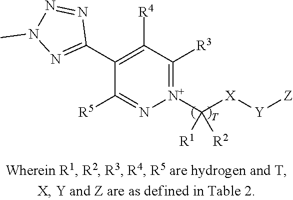 | |

| Com- | | | | |
|---|---|---|---|---|
| pound | | | | |
| num- | | | | |
| ber | X | Y | Z | T |
| 2.001 | —C(O)— | CH₂ | —C(O)OH | 2 |
| 2.002 | —C(O)— | CH₂ | —C(O)NHS(O)₂Me | 2 |
| 2.003 | —C(O)— | CH₂ | —S(O)₂OH | 2 |
| 2.004 | —C(O)— | CH₂ | —P(O)(OH)(OMe) | 2 |
| 2.005 | —C(O)— | CH₂ | —P(O)(OH)(Me) | 2 |
| 2.006 | —C(O)— | CH₂ | —P(O)(OH)(OH) | 2 |
| 2.007 | —NHC(O)— | CH₂ | —C(O)OH | 2 |
| 2.008 | —NHC(O)— | CH₂ | —C(O)NHS(O)₂Me | 2 |
| 2.009 | —NHC(O)— | CH₂ | —S(O)₂OH | 2 |
| 2.010 | —NHC(O)— | CH₂ | —P(O)(OH)(OMe) | 2 |
| 2.011 | —NHC(O)— | CH₂ | —P(O)(OH)(Me) | 2 |
| 2.012 | —NHC(O)— | CH₂ | —P(O)(OH)(OH) | 2 |
| 2.013 | —C(O)NH— | CH₂ | —C(O)OH | 2 |
| 2.014 | —C(O)NH | CH₂ | —C(O)NHS(O)₂Me | 2 |
| 2.015 | —C(O)NH | CH₂ | —S(O)₂OH | 2 |
| 2.016 | —C(O)NH | CH₂ | —P(O)(OH)(OMe) | 2 |
| 2.017 | —C(O)NH | CH₂ | —P(O)(OH)(Me) | 2 |
| 2.018 | —C(O)NH | CH₂ | —P(O)(OH)(OH) | 2 |
| 2.019 | —NHC(O)NH— | CH₂ | —C(O)OH | 2 |
| 2.020 | —NHC(O)NH— | CH₂ | —C(O)NHS(O)₂Me | 2 |
| 2.021 | —NHC(O)NH— | CH₂ | —S(O)₂OH | 2 |
| 2.022 | —NHC(O)NH— | CH₂ | —P(O)(OH)(OMe) | 2 |
| 2.023 | —NHC(O)NH— | CH₂ | —P(O)(OH)(Me) | 2 |
| 2.024 | —NHC(O)NH— | CH₂ | —P(O)(OH)(OH) | 2 |
| 2.025 | —O— | CH₂ | —C(O)OH | 2 |
| 2.026 | —O— | CH₂ | —C(O)NHS(O)₂Me | 2 |
| 2.027 | —O— | CH₂ | —S(O)₂OH | 2 |
| 2.028 | —O— | CH₂ | —P(O)(OH)(OMe) | 2 |
| 2.029 | —O— | CH₂ | —P(O)(OH)(Me) | 2 |
| 2.030 | —O— | CH₂ | —P(O)(OH)(OH) | 2 |
| 2.031 | —S— | CH₂ | —C(O)OH | 2 |
| 2.032 | —S— | CH₂ | —C(O)NHS(O)₂Me | 2 |
| 2.033 | —S— | CH₂ | —S(O)₂OH | 2 |
| 2.034 | —S— | CH₂ | —P(O)(OH)(OMe) | 2 |
| 2.035 | —S— | CH₂ | —P(O)(OH)(Me) | 2 |
| 2.036 | —S— | CH₂ | —P(O)(OH)(OH) | 2 |
| 2.037 | —SO— | CH₂ | —C(O)OH | 2 |
| 2.038 | —SO— | CH₂ | —C(O)NHS(O)₂Me | 2 |
| 2.039 | —SO— | CH₂ | —S(O)₂OH | 2 |
| 2.040 | —SO— | CH₂ | —P(O)(OH)(OMe) | 2 |
| 2.041 | —SO— | CH₂ | —P(O)(OH)(Me) | 2 |
| 2.042 | —SO— | CH₂ | —P(O)(OH)(OH) | 2 |
| 2.043 | —SO₂— | CH₂ | —C(O)OH | 2 |
| 2.044 | —SO₂— | CH₂ | —C(O)NHS(O)₂Me | 2 |
| 2.045 | —SO₂— | CH₂ | —S(O)₂OH | 2 |
| 2.046 | —SO₂— | CH₂ | —P(O)(OH)(OMe) | 2 |
| 2.047 | —SO₂— | CH₂ | —P(O)(OH)(Me) | 2 |
| 2.048 | —SO₂— | CH₂ | —P(O)(OH)(OH) | 2 |
| 2.049 | —NHS(O)₂— | CH₂ | —C(O)OH | 2 |
| 2.050 | —NHS(O)₂ | CH₂ | —C(O)NHS(O)₂Me | 2 |
| 2.051 | —NHS(O)₂ | CH₂ | —S(O)₂OH | 2 |
| 2.052 | —NHS(O)₂ | CH₂ | —P(O)(OH)(OMe) | 2 |
| 2.053 | —NHS(O)₂ | CH₂ | —P(O)(OH)(Me) | 2 |
| 2.054 | —NHS(O)₂ | CH₂ | —P(O)(OH)(OH) | 2 |
| 2.055 | —S(O)₂NH— | CH₂ | —C(O)OH | 2 |
| 2.056 | —S(O)₂NH— | CH₂ | —C(O)NHS(O)₂Me | 2 |
| 2.057 | —S(O)₂NH— | CH₂ | —S(O)₂OH | 2 |
| 2.058 | —S(O)₂NH— | CH₂ | —P(O)(OH)(OMe) | 2 |
| 2.059 | —S(O)₂NH— | CH₂ | —P(O)(OH)(Me) | 2 |
| 2.060 | —S(O)₂NH— | CH₂ | —P(O)(OH)(OH) | 2 |

TABLE 3
This table discloses 60 specific compounds of the formula (T-3):

| | |
|---|---|
| | (T-3) |
| 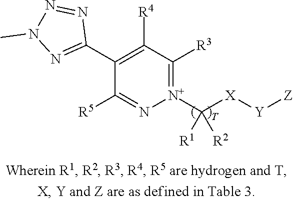 | |

| Com- | | | | |
|---|---|---|---|---|
| pound | | | | |
| num- | | | | |
| ber | X | Y | Z | T |
| 3.001 | —C(O)— | (CH₂)₂ | —C(O)OH | 1 |
| 3.002 | —C(O)— | (CH₂)₂ | —C(O)NHS(O)₂Me | 1 |
| 3.003 | —C(O)— | (CH₂)₂ | —S(O)₂OH | 1 |
| 3.004 | —C(O)— | (CH₂)₂ | —P(O)(OH)(OMe) | 1 |
| 3.005 | —C(O)— | (CH₂)₂ | —P(O)(OH)(Me) | 1 |
| 3.006 | —C(O)— | (CH₂)₂ | —P(O)(OH)(OH) | 1 |
| 3.007 | —NHC(O)— | (CH₂)₂ | —C(O)OH | 1 |
| 3.008 | —NHC(O)— | (CH₂)₂ | —C(O)NHS(O)₂Me | 1 |
| 3.009 | —NHC(O)— | (CH₂)₂ | —S(O)₂OH | 1 |
| 3.010 | —NHC(O)— | (CH₂)₂ | —P(O)(OH)(OMe) | 1 |
| 3.011 | —NHC(O)— | (CH₂)₂ | —P(O)(OH)(Me) | 1 |
| 3.012 | —NHC(O)— | (CH₂)₂ | —P(O)(OH)(OH) | 1 |
| 3.013 | —C(O)NH— | (CH₂)₂ | —C(O)OH | 1 |
| 3.014 | —C(O)NH | (CH₂)₂ | —C(O)NHS(O)₂Me | 1 |
| 3.015 | —C(O)NH | (CH₂)₂ | —S(O)₂OH | 1 |
| 3.016 | —C(O)NH | (CH₂)₂ | —P(O)(OH)(OMe) | 1 |
| 3.017 | —C(O)NH | (CH₂)₂ | —P(O)(OH)(Me) | 1 |
| 3.018 | —C(O)NH | (CH₂)₂ | —P(O)(OH)(OH) | 1 |
| 3.019 | —NHC(O)NH— | (CH₂)₂ | —C(O)OH | 1 |
| 3.020 | —NHC(O)NH— | (CH₂)₂ | —C(O)NHS(O)₂Me | 1 |
| 3.021 | —NHC(O)NH— | (CH₂)₂ | —S(O)₂OH | 1 |
| 3.022 | —NHC(O)NH— | (CH₂)₂ | —P(O)(OH)(OMe) | 1 |
| 3.023 | —NHC(O)NH— | (CH₂)₂ | —P(O)(OH)(Me) | 1 |
| 3.024 | —NHC(O)NH— | (CH₂)₂ | —P(O)(OH)(OH) | 1 |
| 3.025 | —O— | (CH₂)₂ | —C(O)OH | 1 |
| 3.026 | —O— | (CH₂)₂ | —C(O)NHS(O)₂Me | 1 |
| 3.027 | —O— | (CH₂)₂ | —S(O)₂OH | 1 |
| 3.028 | —O— | (CH₂)₂ | —P(O)(OH)(OMe) | 1 |
| 3.029 | —O— | (CH₂)₂ | —P(O)(OH)(Me) | 1 |
| 3.030 | —O— | (CH₂)₂ | —P(O)(OH)(OH) | 1 |
| 3.031 | —S— | (CH₂)₂ | —C(O)OH | 1 |
| 3.032 | —S— | (CH₂)₂ | —C(O)NHS(O)₂Me | 1 |
| 3.033 | —S— | (CH₂)₂ | —S(O)₂OH | 1 |
| 3.034 | —S— | (CH₂)₂ | —P(O)(OH)(OMe) | 1 |
| 3.035 | —S— | (CH₂)₂ | —P(O)(OH)(Me) | 1 |
| 3.036 | —S— | (CH₂)₂ | —P(O)(OH)(OH) | 1 |
| 3.037 | —SO— | (CH₂)₂ | —C(O)OH | 1 |
| 3.038 | —SO— | (CH₂)₂ | —C(O)NHS(O)₂Me | 1 |
| 3.039 | —SO— | (CH₂)₂ | —S(O)₂OH | 1 |
| 3.040 | —SO— | (CH₂)₂ | —P(O)(OH)(OMe) | 1 |
| 3.041 | —SO— | (CH₂)₂ | —P(O)(OH)(Me) | 1 |
| 3.042 | —SO— | (CH₂)₂ | —P(O)(OH)(OH) | 1 |
| 3.043 | —SO₂— | (CH₂)₂ | —C(O)OH | 1 |
| 3.044 | —SO₂— | (CH₂)₂ | —C(O)NHS(O)₂Me | 1 |
| 3.045 | —SO₂— | (CH₂)₂ | —S(O)₂OH | 1 |
| 3.046 | —SO₂— | (CH₂)₂ | —P(O)(OH)(OMe) | 1 |
| 3.047 | —SO₂— | (CH₂)₂ | —P(O)(OH)(Me) | 1 |
| 3.048 | —SO₂— | (CH₂)₂ | —P(O)(OH)(OH) | 1 |
| 3.049 | —NHS(O)₂— | (CH₂)₂ | —C(O)OH | 1 |
| 3.050 | —NHS(O)₂— | (CH₂)₂ | —C(O)NHS(O)₂Me | 1 |
| 3.051 | —NHS(O)₂— | (CH₂)₂ | —S(O)₂OH | 1 |
| 3.052 | —NHS(O)₂— | (CH₂)₂ | —P(O)(OH)(OMe) | 1 |
| 3.053 | —NHS(O)₂— | (CH₂)₂ | —P(O)(OH)(Me) | 1 |
| 3.054 | —NHS(O)₂— | (CH₂)₂ | —P(O)(OH)(OH) | 1 |
| 3.055 | —S(O)₂NH— | (CH₂)₂ | —C(O)OH | 1 |
| 3.056 | —S(O)₂NH— | (CH₂)₂ | —C(O)NHS(O)₂Me | 1 |
| 3.057 | —S(O)₂NH— | (CH₂)₂ | —S(O)₂OH | 1 |
| 3.058 | —S(O)₂NH— | (CH₂)₂ | —P(O)(OH)(OMe) | 1 |
| 3.059 | —S(O)₂NH— | (CH₂)₂ | —P(O)(OH)(Me) | 1 |
| 3.060 | —S(O)₂NH— | (CH₂)₂ | —P(O)(OH)(OH) | 1 |

TABLE 4
This table discloses 60 specific compounds of the formula (T-4):

| | |
|---|---|
| | (T-4) |
| 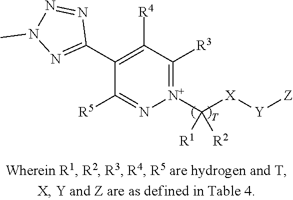 | |

| Com- | | | | |
|---|---|---|---|---|
| pound | | | | |
| num- | | | | |
| ber | X | Y | Z | T |
| 4.001 | —C(O)— | (CH₂)₂ | —C(O)OH | 2 |
| 4.002 | —C(O)— | (CH₂)₂ | —C(O)NHS(O)₂Me | 2 |
| 4.003 | —C(O)— | (CH₂)₂ | —S(O)₂OH | 2 |
| 4.004 | —C(O)— | (CH₂)₂ | —P(O)(OH)(OMe) | 2 |
| 4.005 | —C(O)— | (CH₂)₂ | —P(O)(OH)(Me) | 2 |
| 4.006 | —C(O)— | (CH₂)₂ | —P(O)(OH)(OH) | 2 |
| 4.007 | —NHC(O)— | (CH₂)₂ | —C(O)OH | 2 |
| 4.008 | —NHC(O)— | (CH₂)₂ | —C(O)NHS(O)₂Me | 2 |
| 4.009 | —NHC(O)— | (CH₂)₂ | —S(O)₂OH | 2 |
| 4.010 | —NHC(O)— | (CH₂)₂ | —P(O)(OH)(OMe) | 2 |
| 4.011 | —NHC(O)— | (CH₂)₂ | —P(O)(OH)(Me) | 2 |
| 4.012 | —NHC(O)— | (CH₂)₂ | —P(O)(OH)(OH) | 2 |
| 4.013 | —C(O)NH— | (CH₂)₂ | —C(O)OH | 2 |
| 4.014 | —C(O)NH— | (CH₂)₂ | —C(O)NHS(O)₂Me | 2 |
| 4.015 | —C(O)NH— | (CH₂)₂ | —S(O)₂OH | 2 |
| 4.016 | —C(O)NH— | (CH₂)₂ | —P(O)(OH)(OMe) | 2 |
| 4.017 | —C(O)NH— | (CH₂)₂ | —P(O)(OH)(Me) | 2 |
| 4.018 | —C(O)NH— | (CH₂)₂ | —P(O)(OH)(OH) | 2 |
| 4.019 | —NHC(O)NH— | (CH₂)₂ | —C(O)OH | 2 |
| 4.020 | —NHC(O)NH— | (CH₂)₂ | —C(O)NHS(O)₂Me | 2 |
| 4.021 | —NHC(O)NH— | (CH₂)₂ | —S(O)₂OH | 2 |
| 4.022 | —NHC(O)NH— | (CH₂)₂ | —P(O)(OH)(OMe) | 2 |
| 4.023 | —NHC(O)NH— | (CH₂)₂ | —P(O)(OH)(Me) | 2 |
| 4.024 | —NHC(O)NH— | (CH₂)₂ | —P(O)(OH)(OH) | 2 |
| 4.025 | —O— | (CH₂)₂ | —C(O)OH | 2 |
| 4.026 | —O— | (CH₂)₂ | —C(O)NHS(O)₂Me | 2 |
| 4.027 | —O— | (CH₂)₂ | —S(O)₂OH | 2 |
| 4.028 | —O— | (CH₂)₂ | —P(O)(OH)(OMe) | 2 |
| 4.029 | —O— | (CH₂)₂ | —P(O)(OH)(Me) | 2 |
| 4.030 | —O— | (CH₂)₂ | —P(O)(OH)(OH) | 2 |
| 4.031 | —S— | (CH₂)₂ | —C(O)OH | 2 |
| 4.032 | —S— | (CH₂)₂ | —C(O)NHS(O)₂Me | 2 |
| 4.033 | —S— | (CH₂)₂ | —S(O)₂OH | 2 |
| 4.034 | —S— | (CH₂)₂ | —P(O)(OH)(OMe) | 2 |
| 4.035 | —S— | (CH₂)₂ | —P(O)(OH)(Me) | 2 |
| 4.036 | —S— | (CH₂)₂ | —P(O)(OH)(OH) | 2 |
| 4.037 | —SO— | (CH₂)₂ | —C(O)OH | 2 |
| 4.038 | —SO— | (CH₂)₂ | —C(O)NHS(O)₂Me | 2 |
| 4.039 | —SO— | (CH₂)₂ | —S(O)₂OH | 2 |
| 4.040 | —SO— | (CH₂)₂ | —P(O)(OH)(OMe) | 2 |
| 4.041 | —SO— | (CH₂)₂ | —P(O)(OH)(Me) | 2 |
| 4.042 | —SO— | (CH₂)₂ | —P(O)(OH)(OH) | 2 |
| 4.043 | —SO₂— | (CH₂)₂ | —C(O)OH | 2 |
| 4.044 | —SO₂— | (CH₂)₂ | —C(O)NHS(O)₂Me | 2 |
| 4.045 | —SO₂— | (CH₂)₂ | —S(O)₂OH | 2 |
| 4.046 | —SO₂— | (CH₂)₂ | —P(O)(OH)(OMe) | 2 |
| 4.047 | —SO₂— | (CH₂)₂ | —P(O)(OH)(Me) | 2 |
| 4.048 | —SO₂— | (CH₂)₂ | —P(O)(OH)(OH) | 2 |
| 4.049 | —NHS(O)₂— | (CH₂)₂ | —C(O)OH | 2 |
| 4.050 | —NHS(O)₂— | (CH₂)₂ | —C(O)NHS(O)₂Me | 2 |
| 4.051 | —NHS(O)₂— | (CH₂)₂ | —S(O)₂OH | 2 |
| 4.052 | —NHS(O)₂— | (CH₂)₂ | —P(O)(OH)(OMe) | 2 |
| 4.053 | —NHS(O)₂— | (CH₂)₂ | —P(O)(OH)(Me) | 2 |
| 4.054 | —NHS(O)₂— | (CH₂)₂ | —P(O)(OH)(OH) | 2 |
| 4.055 | —S(O)₂NH— | (CH₂)₂ | —C(O)OH | 2 |
| 4.056 | —S(O)₂NH— | (CH₂)₂ | —C(O)NHS(O)₂Me | 2 |
| 4.057 | —S(O)₂NH— | (CH₂)₂ | —S(O)₂OH | 2 |
| 4.058 | —S(O)₂NH— | (CH₂)₂ | —P(O)(OH)(OMe) | 2 |
| 4.059 | —S(O)₂NH— | (CH₂)₂ | —P(O)(OH)(Me) | 2 |
| 4.060 | —S(O)₂NH— | (CH₂)₂ | —P(O)(OH)(OH) | 2 |

TABLE 5
This table discloses 78 specific compounds of the formula (T-5):

| | |
|---|---|
| | (T-5) |
| 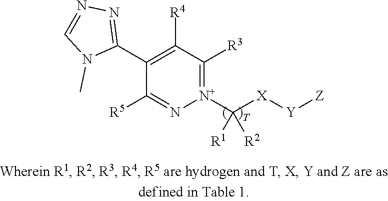 | |

TABLE 6
This table discloses 60 specific compounds of the formula (T-6):

| | |
|---|---|
| | (T-6) |
| 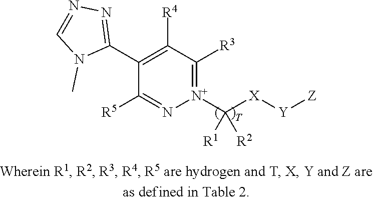 | |

TABLE 7:

| This table discloses 60 specific compounds of the formula (T-7): | |
|---|---|
| 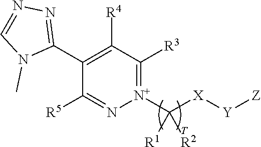 | (T-7) |

Wherein R¹, R², R³, R⁴, R⁵ are hydrogen and T, X, Y
and Z are as defined in Table 3.

TABLE 8:

| This table discloses 60 specific compounds of the formula (T-8): | |
|---|---|
| 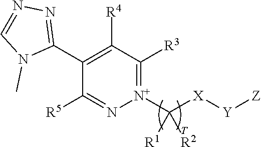 | (T-8) |

Wherein R¹, R², R³, R⁴, R⁵ are hydrogen and T, X, Y
and Z are as defined in Table 4.

TABLE 9:

| This table discloses 78 specific compounds of the formula (T-9): | |
|---|---|
| 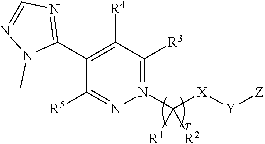 | (T-9) |

Wherein R¹, R², R³, R⁴, R⁵ are hydrogen and T, X, Y
and Z are as defined in Table 1.

TABLE 10:

| This table discloses 60 specific compounds of the formula (T-10): | |
|---|---|
| 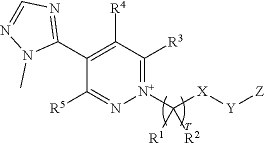 | (T-10) |

Wherein R¹, R², R³, R⁴, R⁵ are hydrogen and T, X, Y
and Z are as defined in Table 2.

TABLE 11:

| This table discloses 60 specific compounds of the formula (T-11): | |
|---|---|
| 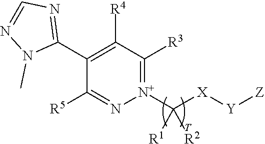 | (T-11) |

Wherein R¹, R², R³, R⁴, R⁵ are hydrogen and T, X, Y
and Z are as defined in Table 3.

TABLE 12:

| This table discloses 60 specific compounds of the formula (T-12): | |
|---|---|
| 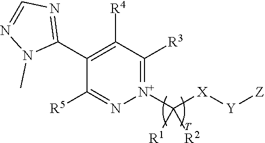 | (T-12) |

Wherein R¹, R², R³, R⁴, R⁵ are hydrogen and T, X, Y
and Z are as defined in Table 4.

TABLE 13:

| This table discloses 78 specific compounds of the formula (T-13): | |
|---|---|
| 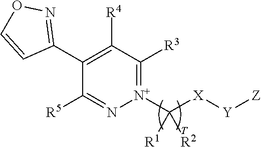 | (T-13) |

Wherein R¹, R², R³, R⁴, R⁵ are hydrogen and T, X, Y
and Z are as defined in Table 1.

TABLE 14:

| This table discloses 60 specific compounds of the formula (T-14): | |
|---|---|
| 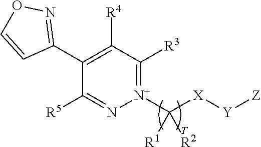 | (T-14) |

Wherein R¹, R², R³, R⁴, R⁵ are hydrogen and T, X, Y
and Z are as defined in Table 2.

TABLE 15:

| This table discloses 60 specific compounds of the formula (T-15): | |
|---|---|
| 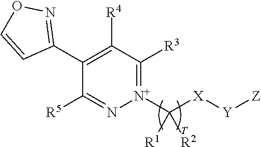 | (T-15) |

Wherein R¹, R², R³, R⁴, R⁵ are hydrogen and T, X, Y
and Z are as defined in Table 3.

TABLE 16:

| This table discloses 60 specific compounds of the formula (T-16): | |
|---|---|
| 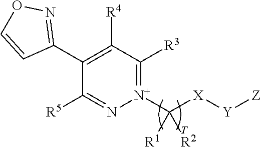 | (T-16) |

Wherein R¹, R², R³, R⁴, R⁵ are hydrogen and T, X, Y
and Z are as defined in Table 4.

TABLE 17:

| This table discloses 78 specific compounds of the formula (T-17): | |
|---|---|
| 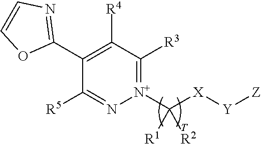 | (T-17) |

Wherein R¹, R², R³, R⁴, R⁵ are hydrogen and T, X, Y and
Z are as defined in Table 1.

TABLE 18:

| This table discloses 60 specific compounds of the formula (T-18): | |
|---|---|
| 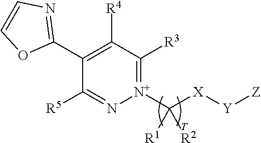 | (T-18) |

Wherein R¹, R², R³, R⁴, R⁵ are hydrogen and T, X, Y
and Z are as defined in Table 2.

TABLE 19:

| This table discloses 60 specific compounds of the formula (T-19): | |
|---|---|
| 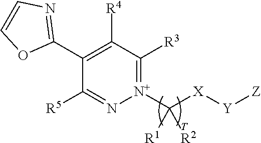 | (T-19) |

Wherein R¹, R², R³, R⁴, R⁵ are hydrogen and T, X, Y
and Z are as defined in Table 3.

TABLE 20:

| This table discloses 60 specific compounds of the formula (T-20): | |
|---|---|
| 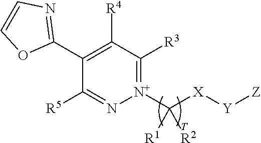 | (T-20) |

Wherein R¹, R², R³, R⁴, R⁵ are hydrogen and T, X, Y and Z
are as defined in Table 4.

TABLE 21:

| This table discloses 78 specific compounds of the formula (T-21): | |
|---|---|
| 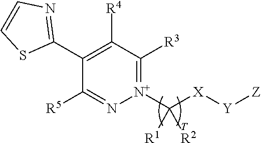 | (T-21) |

Wherein R¹, R², R³, R⁴, R⁵ are hydrogen and T, X, Y
and Z are as defined in Table 1.

TABLE 22
This table discloses 60 specific compounds of the formula (T-22):

| | |
|---|---|
| 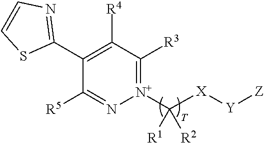 | (T-22) |

Wherein R¹, R², R³, R⁴, R⁵ are hydrogen and T, X, Y and Z are as defined in Table 2.

TABLE 23
This table discloses 60 specific compounds of the formula (T-23):

| | |
|---|---|
| 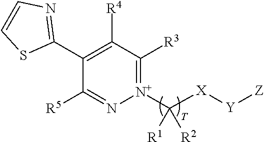 | (T-23) |

Wherein R¹, R², R³, R⁴, R⁵ are hydrogen and T, X, Y and Z are as defined in Table 3.

TABLE 24
This table discloses 60 specific compounds of the formula (T-24):

| | |
|---|---|
| 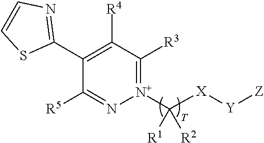 | (T-24) |

Wherein R¹, R², R³, R⁴, R⁵ are hydrogen and T, X, Y and Z are as defined in Table 4.

TABLE 25
This table discloses 78 specific compounds of the formula (T-25):

| | |
|---|---|
| 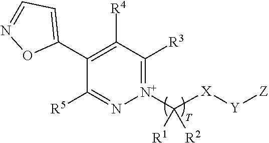 | (T-25) |

Wherein R¹, R², R³, R⁴, R⁵ are hydrogen and T, X, Y and Z are as defined in Table 1.

TABLE 26
This table discloses 60 specific compounds of the formula (T-26):

| | |
|---|---|
| 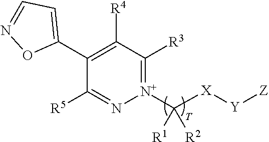 | (T-26) |

Wherein R¹, R², R³, R⁴, R⁵ are hydrogen and T, X, Y and Z are as defined in Table 2.

TABLE 27
This table discloses 60 specific compounds of the formula (T-27):

| | |
|---|---|
| 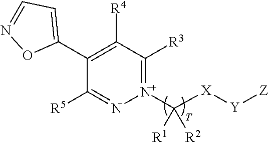 | (T-27) |

Wherein R¹, R², R³, R⁴, R⁵ are hydrogen and T, X, Y and Z are as defined in Table 3.

TABLE 28
This table discloses 60 specific compounds of the formula (T-28):

| | |
|---|---|
| 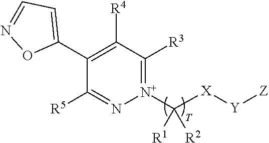 | (T-28) |

Wherein R¹, R², R³, R⁴, R⁵ are hydrogen and T, X, Y and Z are as defined in Table 4.

TABLE 29
This table discloses 78 specific compounds of the formula (T-29):

| | |
|---|---|
| 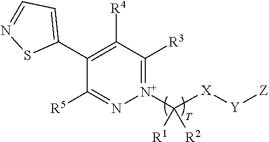 | (T-29) |

Wherein R¹, R², R³, R⁴, R⁵ are hydrogen and T, X, Y and Z are as defined in Table 1.

TABLE 30
This table discloses 60 specific compounds of the formula (T-30):

| | |
|---|---|
| 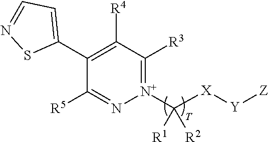 | (T-30) |

Wherein R¹, R², R³, R⁴, R⁵ are hydrogen and T, X, Y and Z are as defined in Table 2.

TABLE 31
This table discloses 60 specific compounds of the formula (T-31):

| | |
|---|---|
| 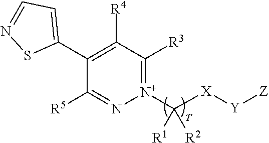 | (T-31) |

Wherein R¹, R², R³, R⁴, R⁵ are hydrogen and T, X, Y and Z are as defined in Table 3.

TABLE 32
This table discloses 60 specific compounds of the formula (T-32):

| | |
|---|---|
| 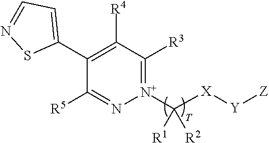 | (T-32) |

Wherein R¹, R², R³, R⁴, R⁵ are hydrogen and T, X, Y and Z are as defined in Table 4.

TABLE 33
This table discloses 78 specific compounds of the formula (T-33):

| | |
|---|---|
| 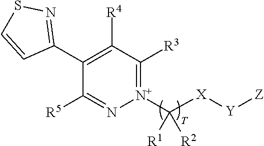 | (T-33) |

Wherein R¹, R², R³, R⁴, R⁵ are hydrogen and T, X, Y and Z are as defined in Table 1.

TABLE 34
This table discloses 60 specific compounds of the formula (T-34):

| | |
|---|---|
| 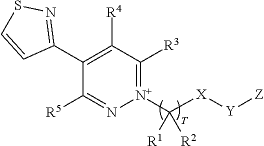 | (T-34) |

Wherein R¹, R², R³, R⁴, R⁵ are hydrogen and T, X, Y and Z are as defined in Table 2.

TABLE 35
This table discloses 60 specific compounds of the formula (T-35):

| | |
|---|---|
| 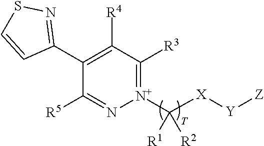 | (T-35) |

Wherein R¹, R², R³, R⁴, R⁵ are hydrogen and T, X, Y and Z are as defined in Table 3.

TABLE 36
This table discloses 60 specific compounds of the formula (T-36):

| | |
|---|---|
| 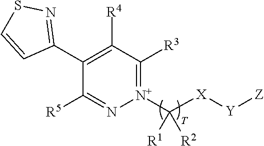 | (T-36) |

Wherein R¹, R², R³, R⁴, R⁵ are hydrogen and T, X, Y and Z are as defined in Table 4.

TABLE 37
This table discloses 78 specific compounds of the formula (T-37):

(T-37)

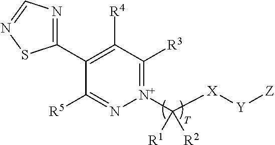

Wherein R¹, R², R³, R⁴, R⁵ are hydrogen and T, X, Y and Z are as defined in Table 1.

TABLE 38
This table discloses 60 specific compounds of the formula (T-38):

(T-38)

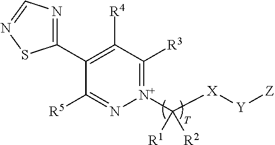

Wherein R¹, R², R³, R⁴, R⁵ are hydrogen and T, X, Y and Z are as defined in Table 2.

TABLE 39
This table discloses 60 specific compounds of the formula (T-39):

(T-39)

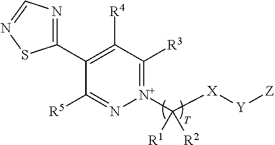

Wherein R¹, R², R³, R⁴, R⁵ are hydrogen and T, X, Y and Z are as defined in Table 3.

TABLE 40
This table discloses 60 specific compounds of the formula (T-40):

(T-40)

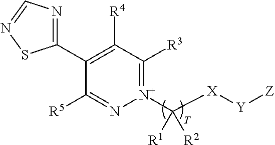

Wherein R¹, R², R³, R⁴, R⁵ are hydrogen and T, X, Y and Z are as defined in Table 4.

TABLE 41
This table discloses 78 specific compounds of the formula (T-41):

(T-41)

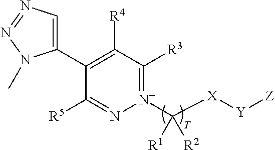

Wherein R¹, R², R³, R⁴, R⁵ are hydrogen and T, X, Y and Z are as defined in Table 1.

TABLE 42
This table discloses 60 specific compounds of the formula (T-42):

(T-42)

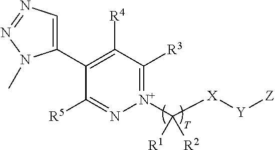

Wherein R¹, R², R³, R⁴, R⁵ are hydrogen and T, X, Y and Z are as defined in Table 2.

TABLE 43
This table discloses 60 specific compounds of the formula (T-43):

(T-43)

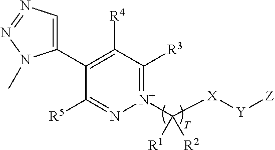

Wherein R¹, R², R³, R⁴, R⁵ are hydrogen and T, X, Y and Z are as defined in Table 3.

TABLE 44
This table discloses 60 specific compounds of the formula (T-44):

(T-44)

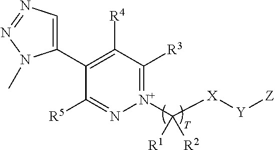

Wherein R¹, R², R³, R⁴, R⁵ are hydrogen and T, X, Y and Z are as defined in Table 4.

TABLE 45
This table discloses 78 specific compounds of the formula (T-45):

(T-45)

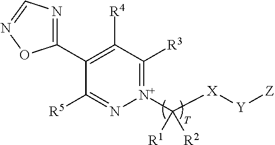

Wherein R¹, R², R³, R⁴, R⁵ are hydrogen and T, X, Y and Z are as defined in Table 1.

TABLE 46
This table discloses 60 specific compounds of the formula (T-46):

(T-46)

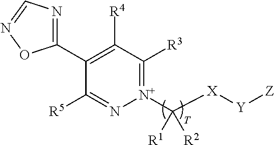

Wherein R¹, R², R³, R⁴, R⁵ are hydrogen and T, X, Y and Z are as defined in Table 2.

TABLE 47
This table discloses 60 specific compounds of the formula (T-47):

(T-47)

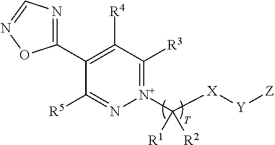

Wherein R¹, R², R³, R⁴, R⁵ are hydrogen and T, X, Y and Z are as defined in Table 3.

TABLE 48
This table discloses 60 specific compounds of the formula (T-48):

(T-48)

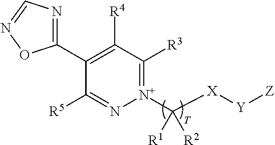

Wherein R¹, R², R³, R⁴, R⁵ are hydrogen and T, X, Y and Z are as defined in Table 4.

TABLE 49
This table discloses 78 specific compounds of the formula (T-49):

(T-49)

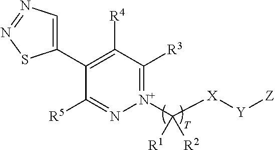

Wherein R¹, R², R³, R⁴, R⁵ are hydrogen and T, X, Y and Z are as defined in Table 1.

TABLE 50
This table discloses 60 specific compounds of the formula (T-50):

(T-50)

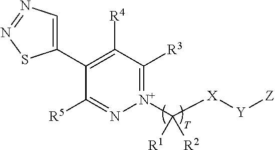

Wherein R¹, R², R³, R⁴, R⁵ are hydrogen and T, X, Y and Z are as defined in Table 2.

TABLE 51
This table discloses 60 specific compounds of the formula (T-51):

(T-51)

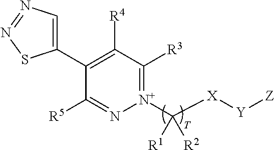

Wherein R¹, R², R³, R⁴, R⁵ are hydrogen and T, X, Y and Z are as defined in Table 3.

TABLE 52
This table discloses 60 specific compounds of the formula (T-52):

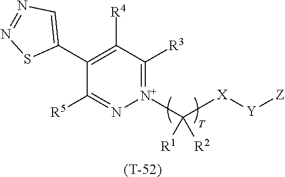

Wherein R¹, R², R³, R⁴, R⁵ are hydrogen and T, X, Y and Z are as defined in Table 4.

TABLE 53
This table discloses 78 specific compounds of the formula (T-53):

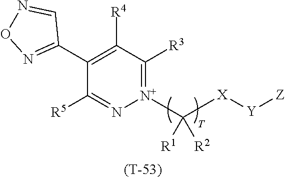

Wherein R¹, R², R³, R⁴, R⁵ are hydrogen and T, X, Y and Z are as defined in Table 1.

TABLE 54
This table discloses 60 specific compounds of the formula (T-54):

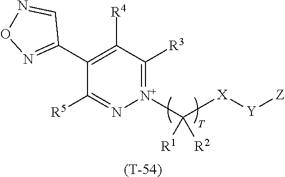

Wherein R¹, R², R³, R⁴, R⁵ are hydrogen and T, X, Y and Z are as defined in Table 2.

TABLE 55
This table discloses 60 specific compounds of the formula (T-55):

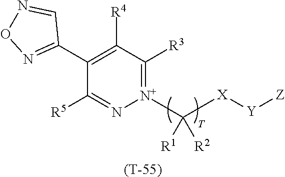

Wherein R¹, R², R³, R⁴, R⁵ are hydrogen and T, X, Y and Z are as defined in Table 3.

TABLE 56
This table discloses 60 specific compounds of the formula (T-56):

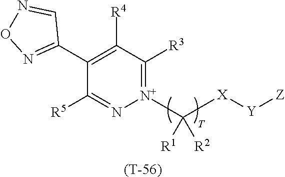

Wherein R¹, R², R³, R⁴, R⁵ are hydrogen and T, X, Y and Z are as defined in Table 4.

TABLE 57
This table discloses 78 specific compounds of the formula (T-57):

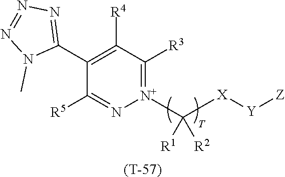

Wherein R¹, R², R³, R⁴, R⁵ are hydrogen and T, X, Y and Z are as defined in Table 1.

TABLE 58
This table discloses 60 specific compounds of the formula (T-58):

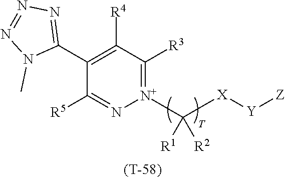

Wherein R¹, R², R³, R⁴, R⁵ are hydrogen and T, X, Y and Z are as defined in Table 2.

TABLE 59
This table discloses 60 specific compounds of the formula (T-59):

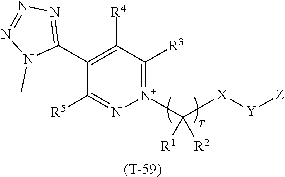

Wherein R¹, R², R³, R⁴, R⁵ are hydrogen and T, X, Y and Z are as defined in Table 3.

TABLE 60
This table discloses 60 specific compounds of the formula (T-60):

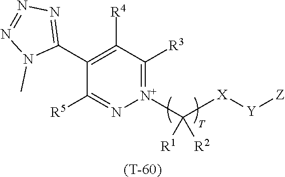

Wherein R¹, R², R³, R⁴, R⁵ are hydrogen and T, X, Y and Z are as defined in Table 4.

TABLE 61
This table discloses 78 specific compounds of the formula (T-61):

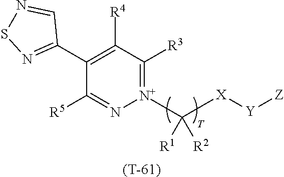

Wherein R¹, R², R³, R⁴, R⁵ are hydrogen and T, X, Y and Z are as defined in Table 1.

TABLE 62
This table discloses 60 specific compounds of the formula (T-62):

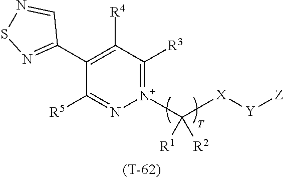

Wherein R¹, R², R³, R⁴, R⁵ are hydrogen and T, X, Y and Z are as defined in Table 2.

TABLE 63
This table discloses 60 specific compounds of the formula (T-63):

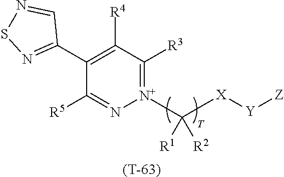

Wherein R¹, R², R³, R⁴, R⁵ are hydrogen and T, X, Y and Z are as defined in Table 3.

TABLE 64
This table discloses 60 specific compounds of the formula (T-64):

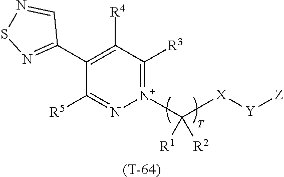

Wherein R¹, R², R³, R⁴, R⁵ are hydrogen and T, X, Y and Z are as defined in Table 4.

TABLE 65
This table discloses 78 specific compounds of the formula (T-65):

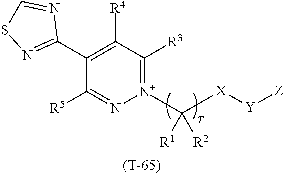

Wherein R¹, R², R³, R⁴, R⁵ are hydrogen and T, X, Y and Z are as defined in Table 1.

TABLE 66
This table discloses 60 specific compounds of the formula (T-66):

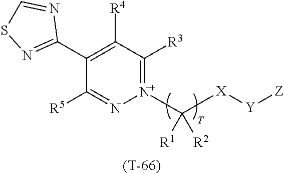

Wherein R¹, R², R³, R⁴, R⁵ are hydrogen and T, X, Y and Z are as defined in Table 2.

TABLE 67
This table discloses 60 specific compounds of the formula (T-67):

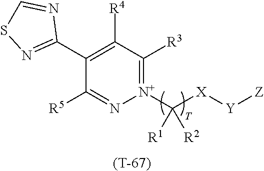

Wherein R¹, R², R³, R⁴, R⁵ are hydrogen and T, X, Y and Z are as defined in Table 3.

TABLE 68
This table discloses 60 specific compounds of the formula (T-68):

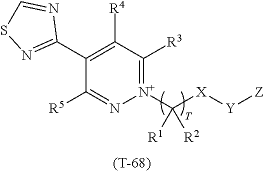

Wherein R¹, R², R³, R⁴, R⁵ are hydrogen and T, X, Y and Z are as defined in Table 4.

TABLE 69
This table discloses 78 specific compounds of the formula (T-69):

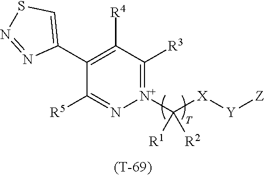

Wherein R^{1,} R², R³, R⁴, R⁵ are hydrogen and T, X, Y and Z are as defined in Table 1.

TABLE 70
This table discloses 60 specific compounds of the formula (T-70):

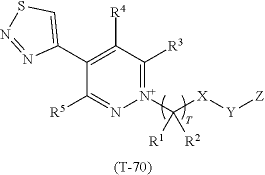

Wherein R¹, R², R³, R⁴, R⁵ are hydrogen and T, X, Y and Z are as defined in Table 2.

TABLE 71
This table discloses 60 specific compounds of the formula (T-71):

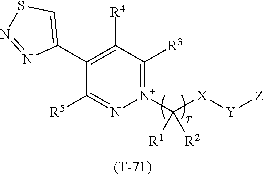

Wherein R¹, R², R³, R⁴, R⁵ are hydrogen and T, X, Y and Z are as defined in Table 3.

TABLE 72
This table discloses 60 specific compounds of the formula (T-72):

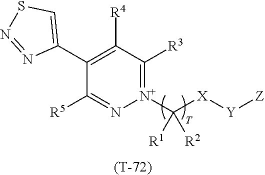

Wherein R¹, R², R³, R⁴, R⁵ are hydrogen and T, X, Y and Z are as defined in Table 4.

TABLE 73
This table discloses 78 specific compounds of the formula (T-73):

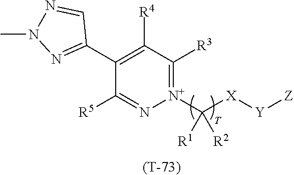

Wherein R¹, R², R³, R⁴, R⁵ are hydrogen and T, X, Y and Z are as defined in Table 1.

TABLE 74
This table discloses 60 specific compounds of the formula (T-74):

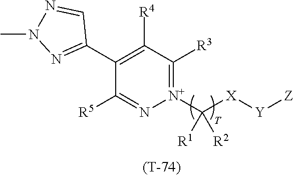

Wherein R¹, R², R³, R⁴, R⁵ are hydrogen and T, X, Y and Z are as defined in Table 2.

TABLE 75
This table discloses 60 specific compounds of the formula (T-75):

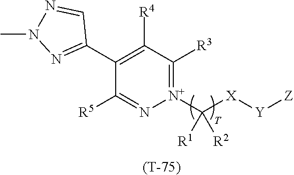

Wherein R¹, R², R³, R⁴, R⁵ are hydrogen and T, X, Y and Z are as defined in Table 3.

TABLE 76
This table discloses 60 specific compounds of the formula (T-76):

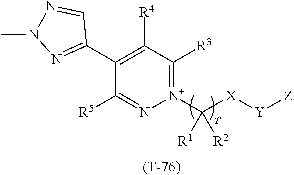

Wherein R¹, R², R³, R⁴, R⁵ are hydrogen and T, X, Y and Z are as defined in Table 4.

TABLE 77
This table discloses 78 specific compounds of the formula (T-77):

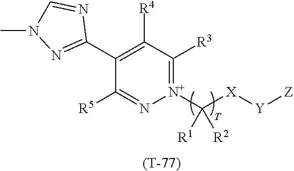

Wherein R¹, R², R³, R⁴, R⁵ are hydrogen and T, X, Y and Z are as defined in Table 1.

TABLE 78
This table discloses 60 specific compounds of the formula (T-78):

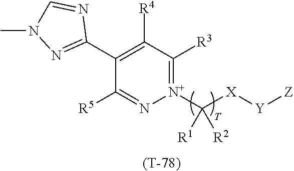

Wherein R¹, R², R³, R⁴, R⁵ are hydrogen and T, X, Y and Z are as defined in Table 2.

TABLE 79
This table discloses 60 specific compounds of the formula (T-79):

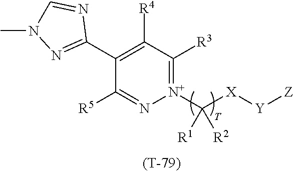

Wherein R¹, R², R³, R⁴, R⁵ are hydrogen and T, X, Y and Z are as defined in Table 3.

TABLE 80
This table discloses 60 specific compounds of the formula (T-80):

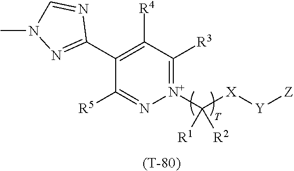

wherein R¹, R², R³, R⁴, R⁵ are hydrogen and T, X, Y and Z are as defined in Table 4.

The compounds of the present invention may be prepared according to the following schemes in which the substituents m, r, T, A, X, Z, R¹, R², R^{1a}, R^{2b}, R³, R⁴, R⁵, R⁶, R⁷, R^{7a}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ are as defined hereinbefore unless explicitly stated otherwise. The compounds of the preceding Tables 1 to 80 may thus be obtained in an analogous manner.

The compounds of formula (I) may be prepared by the alkylation of compounds of formula (X), wherein R³, R⁴, R⁵ and A are as defined for compounds of formula (I), with a suitable alkylating agent of formula (W), wherein R¹, R², T, X, Y and Z are as defined for compounds of formula (I) and LG is a suitable leaving group, for example, halide or pseudohalide such as triflate, mesylate or tosylate, in a suitable solvent at a suitable temperature, as described in reaction scheme 1. Example conditions include stirring a compound of formula (X) with an alkylating agent of formula (W) in a solvent, or mixture of solvents, such as acetone, dichloromethane, dichloroethane, N,N-dimethylformamide, acetonitrile, 1,4-dioxane, water, acetic acid or trifluroacetic acid at a temperature between −78° C. and 150° C. An alkylating agent of formula (W) may include, but is not limited to, ethyl 2-(2-chloroacetamido)acetate, methyl 2-(2-chloroacetamido)acetate, methyl 2-[(2-bromoacetyl)amino]acetate, 2-[(2-chloroacetyl)amino]acetic acid, 2-[(2-bromoacetyl)amino]acetic acid, (2-bromoethoxy)acetic acid, 2-(2-chloroethoxy)acetic acid, ethyl 2-chloroethoxyl acetic acid, methyl 2-chloroethoxyl acetic acid, methyl 2-(3-chloropropanoylamino)acetate, 2-(3-chloropropanoylamino)acetic acid, methyl 2-((2-chloroethyl)sulfonyl)acetate and methyl 2-(2-chloroethylsulfonylamino)acetate. Such alkylating agents and related compounds are either known in the literature or may be prepared by known literature methods. Compounds of formula (I) which may be described as esters of N-alkyl acids, which include, but are not limited to, esters of carboxylic acids, phosphonic acids, phosphinic acids, sulfonic acids and sulfinic acids, may be subsequently partially or fully hydrolysed by treatment with a suitable reagent, for example, aqueous hydrochloric acid or trimethylsilyl bromide, in a suitable solvent at a suitable temperature between 0° C. and 100° C.

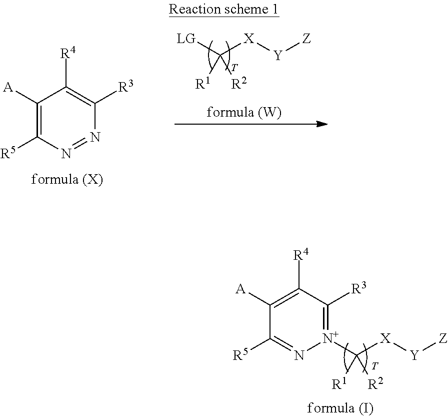

Furthermore, compounds of formula (I) may be prepared by reacting compounds of formula (X), wherein R³, R⁴, R⁵ and A are as defined for compounds of formula (I), with a suitable alcohol of formula (WW), wherein R¹, R², T, X, Y and Z are as defined for compounds of formula (I), under Mitsunobu-type conditions such as those reported by Petit et al, Tet. Lett. 2008, 49 (22), 3663. Suitable phosphines include triphenylphosphine, suitable azodicarboxylates include diisopropylazodicarboxylate and suitable acids include fluoroboric acid, triflic acid and bis(trifluoromethylsulfonyl)amine, as described in reaction scheme 2. Such alcohols are either known in the literature or may be prepared by known literature methods.

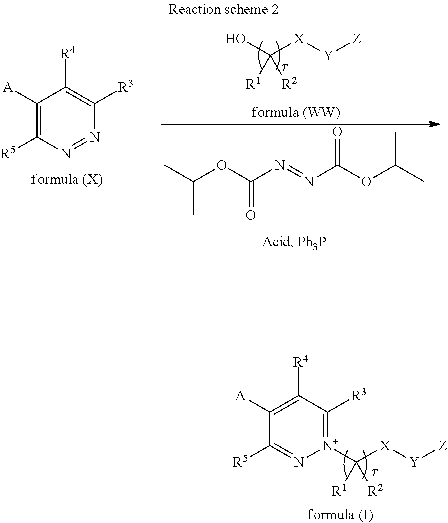

Compounds of formula (I) may also be prepared by reacting compounds of formula (C), wherein R³, R⁴, R⁵ and A are as defined for compounds of formula (I), with a hydrazine of formula (D) in a suitable solvent or mixture of solvents, in the presence of a suitable acid at a suitable temperature, between −78° C. and 150° C., as described in reaction scheme 3. Suitable solvents, or mixtures thereof, include, but are not limited to, alcohols, such as methanol, ethanol and isopropanol, water, aqueous hydrochloric acid, aqueous sulfuric acid, acetic acid and trifluoroacetic acid. Hydrazine compounds of formula (D), for example 2,2-dimethylpropyl 2-hydrazinoethanesulfonate, are either known in the literature or may be prepared by known literature procedures.

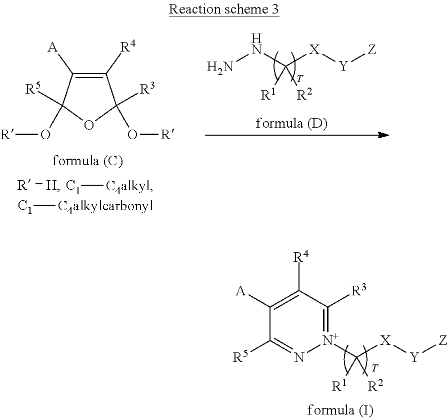

Compounds of formula (C) may be prepared by reacting compounds of formula (G), wherein R³, R⁴, R⁵ and A are as defined for compounds of formula (I), with an oxidising agent in a suitable solvent at a suitable temperature, between −78° C. and 150° C., optionally in the presence of a suitable base, as described in reaction scheme 4. Suitable oxidising agents include, but are not limited to, bromine and suitable solvents include, but are not limited to alcohols such as methanol, ethanol and isopropanol. Suitable bases include, but are not limited to, sodium bicarbonate, sodium carbonate, potassium bicarbonate, potassium carbonate and potassium acetate. Similar reactions are known in the literature (for example Hufford, D. L.; Tarbell, D. S.; Koszalka, T. R. J. Amer. Chem. Soc., 1952, 3014). Furans of formula (G) are known in the literature or may be prepared using literature methods. Example methods include, but are not limited to, transition metal cross-couplings such as Stille (for example Farina, V.; Krishnamurthy, V.; Scott, W. J. Organic Reactions, Vol. 50. 1997, and Gazzard, L. et al. J. Med. Chem., 2015, 5053), Suzuki-Miyaura (for example Ando, S.; Matsunaga, H.; Ishizuka, T. J. Org. Chem. 2017, 1266-1272, and Ernst, J. B.; Rakers, L.; Glorius, F. Synthesis, 2017, 260), Negishi (for example Yang, Y.; Oldenhius, N. J.; Buchwald, S. L. Angew. Chem. Int. Ed. 2013, 615, and Braendvang, M.; Gundersen, L. Bioorg. Med. Chem. 2005, 6360), and Kumada (for example Heravi, M. M.; Hajiabbasi, P. Monatsh. Chem., 2012, 1575). The coupling partners may be selected with reference to the specific cross-coupling reaction and target product. Transition metal catalysts, ligands, bases, solvents and temperatures may be selected with reference to the desired cross-coupling and are known in the literature. Cross-coupling reactions using pseudo halogens, including but not limited to, triflates, mesylates, tosylates and anisoles, may also be achieved under related conditions.

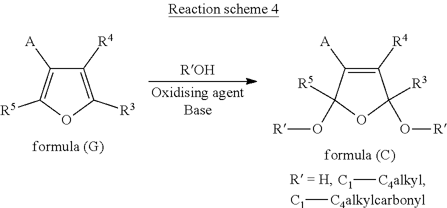

In another approach a compound of formula (I), wherein R¹, R², R³, R⁴, R⁵, A, T, X, Y and Z are as defined for compounds of formula (I), may be prepared from a compound of formula (R) and an oxidant, in a suitable solvent at a suitable temperature, as outlined in reaction scheme 5. Example oxidants include, but are not limited to, 2,3-dichloro-5,6-dicyano-1,4-benzoquinone, tetrachloro-p-benzoquinone, potassium permanganate, manganese dioxide, 2,2,6,6-tetramethyl-1-piperidinyloxy and bromine. Related reactions are known in the literature.

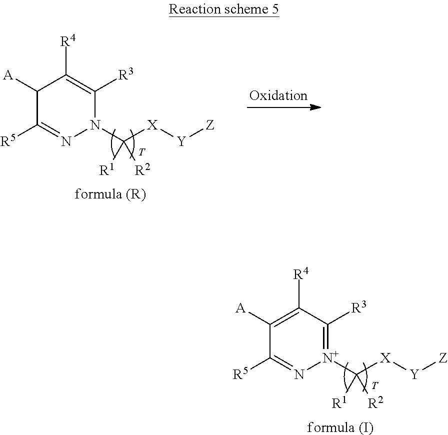

A compound of formula (R), wherein R¹, R², R³, R⁴, R⁵, A, T, X, Y and Z are as defined for compounds of formula (I), may be prepared from a compound of formula (S) and an organometallic of formula (T), which includes, but is not limited to, organomagnesium, organolithium, organocopper and organozinc reagents (M′), in a suitable solvent at a suitable temperature, optionally in the presence of an additional transition metal additive, as outlined in reaction scheme 6. Example conditions include treating a compound of formula (S) with a Grignard of formula (T), in the presence of 0.05-100% copper iodide, in a solvent such as tetrahydrofuran at a temperature between −78° C. and 100° C. Organometallics of formula (T) are known in the literature, or may be prepared by known literature methods. Compounds of formula (S) may be prepared by analogous reactions to those for the preparation of compounds of formula (I).

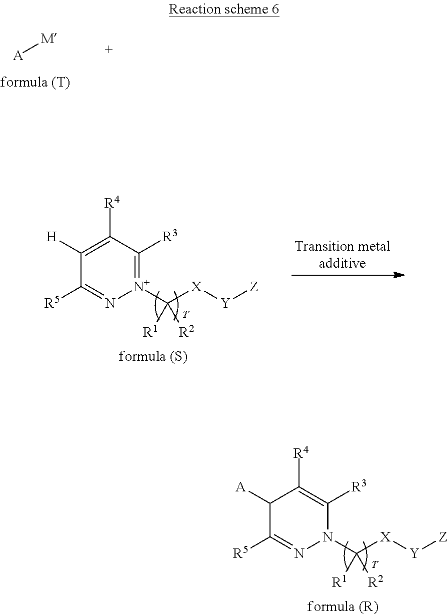

Biaryl pyridazines of formula (X) are known in the literature or may be prepared using literature methods. Example methods include, but are not limited to, the transition metal cross-coupling of compounds of formula (H) and formula (J), or alternatively compounds of formula (K) and formula (L), in which compounds of formula (J) and formula (L) are either an organostannane, organoboronic acid or ester, organotrifluoroborate, organomagnesium, organocopper or organozinc (M), as outlined in reaction scheme 7.

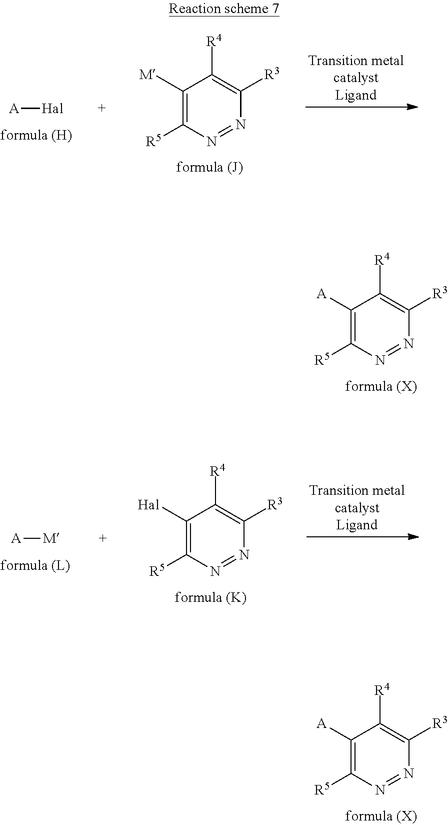

Hal is defined as a halogen or pseudo halogen, for example triflate, mesylate and tosylate. Such cross-couplings include Stille (for example Sauer, J.; Heldmann, D. K. Tetrahedron, 1998, 4297), Suzuki-Miyaura (for example Luebbers, T.; Flohr, A.; Jolidon, S.; David-Pierson, P.; Jacobsen, H.; Ozmen, L.; Baumann, K. Bioorg. Med. Chem. Lett., 2011, 6554), Negishi (for example Imahori, T.; Suzawa, K.; Kondo, Y. Heterocycles, 2008, 1057), and Kumada (for example Heravi, M. M.; Hajiabbasi, P. Monatsh. Chem., 2012, 1575). The coupling partners may be selected with reference to the specific cross-coupling reaction and target product. Transition metal catalysts, ligands, bases, solvents and temperatures may be selected with reference to the desired cross-coupling and are known in the literature. Compounds of formula (H), formula (K) and formula (L) are known in the literature, or may be prepared by known literature methods.

An organometallic of formula (J), which is either an organostannane, organoboronic acid or ester, organotrifluoroborate, organomagnesium, organocopper or organozinc (M′), may be prepared from a compound of formula (XX), wherein R³, R⁴ and R⁵ are as defined for compounds of formula (I), by metallation, as outlined in reaction scheme 8. Similar reactions are known in the literature (for example Ramphal et al, WO2015153683, Unsinn et al., Organic Letters, 15(5), 1128-1131; 2013, Sadler et al., Organic & Biomolecular Chemistry, 12(37), 7318-7327; 2014. Alternatively, an organometallic of formula (J) may be prepared from compounds of formula (K), wherein R³, R⁴, R⁵ are as defined for compounds of formula (I), and Hal is defined as a halogen or pseudo halogen, for example triflate, mesylate and tosylate, as described in scheme 8. Example conditions to prepare an organostannane of formula (J) include treatment of a compound of formula (K) with lithium tributyl tin in an appropriate solvent at an appropriate temperature (for example see WO 2010038465). Example conditions to prepare an organoboronic acid or ester of formula (J) include treatment of a compound of formula (K) with bis(pinacolato)diboron, in the presence of an appropriate transition metal catalyst, appropriate ligand, appropriate base, in an appropriate solvent at an appropriate temperature (for example KR 2015135626). Compounds of formula (K) and formula (XX) are either known in the literature or can be prepared by known methods.

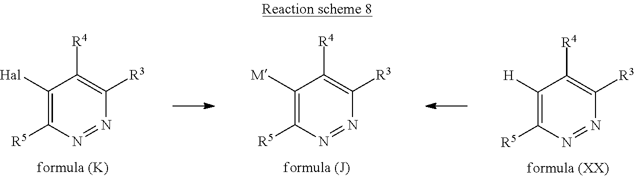

In another approach, an organometallic of formula (J), in which M is either an organostannane or organoboronic acid or ester, may be prepared from a compound of formula (N) and a compound of formula (O), wherein R³, R⁴ and R⁵ are as defined for compounds of formula (I), as outlined in reaction scheme 9. Examples of such a reaction are known in the literature, for example, Helm et al., Org. and Biomed. Chem., 2006, 4 (23), 4278, Sauer et al., Eur. J. Org. Chem., 1998, 12, 2885, and Helm, M. D.; Moore, J. E.; Plant, A.; Harrity, J. P. A., Angew. Chem. Int. Ed., 2005, 3889. Compounds of formula (N) and formula (O) are known in the literature.

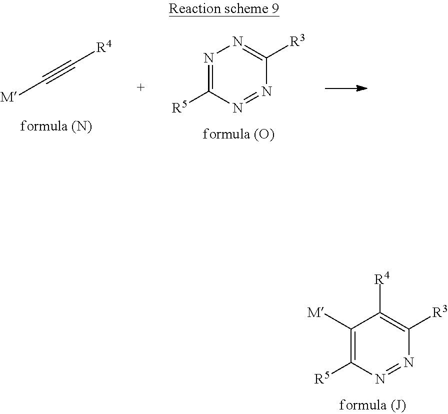

Compounds of formula (X), wherein R³, R⁴, R⁵ and A are as previously defined, may be prepared from compounds of formula (P) and formula (O), in an appropriate solvent, at an appropriate temperature, as outlined in reaction scheme 10. Examples of such a reaction are known in the literature, for example, Sauer et al., Eur. J. Org. Chem., 1998, 12, 2885. Compounds of formula (P) are known in the literature, or may be prepared by known methods.

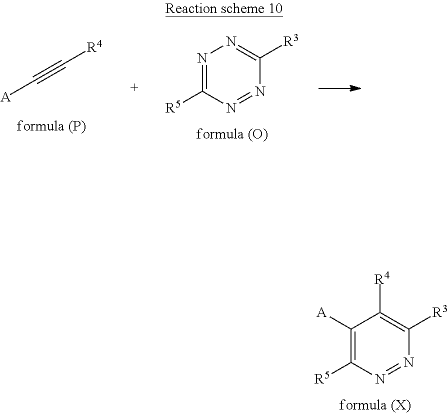

In a further approach a compound of formula (X), wherein R³, R⁴, R⁵ and A are as defined for compounds of formula (I), may be prepared from compounds of formula (C) and hydrazine, in an appropriate solvent, at an appropriate temperature, as outlined in reaction scheme 11. This reaction may also optionally be performed in the presence of an acid, for example aqueous sulfuric acid or aqueous hydrochloric acid. Similar reactions are known in the literature (for example DE 102005029094, and Chen, B.; Bohnert, T.; Zhou, X.; Dedon, P. C. Chem. Res. Toxicol., 2004, 1406). Compounds of formula (C) may be prepared as previously outlined.

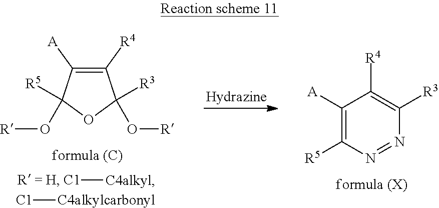

In an additional approach, outlined in scheme 12, biaryl pyridazines of formula (X) may be prepared by classical ring synthesis approaches starting from a compound of formula (ZZ), wherein R³, R⁴ and R⁵ are as defined for compounds of formula (I) and Q is a functional group which can be converted through one or more chemical steps into a 5-membered heteroaryl A, wherein A is as defined for compounds of formula (I). Such functional groups include, but are not limited to, acid, ester, nitrile, amide, thioamide and ketone. Related transformations are known in the literature. Substituted pyridazines may be prepared using methodology outlined in the literature.

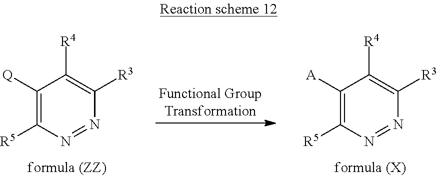

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). For water-soluble compounds, soluble liquids, water-soluble concentrates or water soluble granules are preferred. Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

| | |
|---|---|
| active ingredient: | 1 to 95%, preferably 60 to 90% |
| surface-active agent: | 1 to 30%, preferably 5 to 20% |
| liquid carrier: | 1 to 80%, preferably 1 to 35% |

### Dusts:

| | |
|---|---|
| active ingredient: | 0.1 to 10%, preferably 0.1 to 5% |
| solid carrier: | 99.9 to 90%, preferably 99.9 to 99% |

### Suspension Concentrates:

| | |
|---|---|
| active ingredient: | 5 to 75%, preferably 10 to 50% |
| water: | 94 to 24%, preferably 88 to 30% |
| surface-active agent: | 1 to 40%, preferably 2 to 30% |

### Wettable Powders:

| | |
|---|---|
| active ingredient: | 0.5 to 90%, preferably 1 to 80% |
| surface-active agent: | 0.5 to 20%, preferably 1 to 15% |
| solid carrier: | 5 to 95%, preferably 15 to 90% |

### Granules:

| | |
|---|---|
| active ingredient: | 0.1 to 30%, preferably 0.1 to 15% |
| solid carrier: | 99.5 to 70%, preferably 97 to 85% |

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of formula (I) can be used in combination with one or more other herbicides to provide various herbicidal mixtures. Specific examples of such mixtures include (wherein “I” represents a compound of formula (I)):—I+acetochlor; I+acifluorfen-sodium; I+aclonifen; I+alachlor; I+alloxydim; I+ametryn; I+amicarbazone; I+amidosulfuron; I+aminocyclopyrachlor; I+aminopyralid; I+amitrole; I+asulam; I+atrazine; I+bensulfuron-methyl; I+bentazone; I+bicyclopyrone; I+bifenox; I+bispyribac-sodium; I+bromacil; I+bromoxynil; I+butafenacil; I+cafenstrole; I+carfentrazone-ethyl; I+chlorimuron-ethyl; I+chlorotoluron; I+cinosulfuron; I+clethodim; I+clodinafop-propargyl; I+clomazone; I+clopyralid; I+cyhalofop-butyl; I+2,4-D (including the choline salt and 2-ethylhexyl ester thereof); I+daimuron; I+desmedipham; I+dicamba (including the aluminum, aminopropyl, bis-aminopropylmethyl, choline, diglycolamine, dimethylamine, dimethylammonium, potassium and sodium salts thereof); I+diclofop-methyl; I+difenzoquat; I+diflufenican; I+diflufenzopyr; I+dimethachlor; I+dimethenamid-P; I+diquat dibromide; I+diuron; I+esprocarb; I+ethofumesate; I+fenoxaprop-P-ethyl; I+fenquinotrione; I+flazasulfuron; I+florasulam; I+fluazifop-P-butyl; I+flucarbazone-sodium; I+flufenacet; I+flumetralin; I+flumetsulam; I+flumioxazin; I+flupyrsulfuron-methyl-sodium; I+fluroxypyr-meptyl; I+fluthiacet-methyl; I+fomesafen; I+foramsulfuron; I+glufosinate (including the ammonium salt thereof); I+glyphosate (including the diammonium, isopropylammonium and potassium salts thereof); I+halauxifen-methyl; I+halosulfuron-methyl; I+haloxyfop-methyl; I+hexazinone; I+imazamox; I+imazapic; I+imazapyr; I+imazaquin; I+imazethapyr; I+indaziflam; I+iodosulfuron-methyl-sodium; I+iofensulfuron; I+iofensulfuron-sodium; I+ioxynil; I+ipfencarbazone; I+isoxaben; I+isoxaflutole; I+lactofen; I+linuron; I+mecoprop-P; I+mefenacet; I+mesosulfuron; I+mesosulfuron-methyl; I+mesotrione; I+metamitron; I+metobromuron; I+metolachlor; I+metoxuron; I+metribuzin; I+metsulfuron; I+molinate; I+napropamide; I+nicosulfuron; I+norflurazon; I+orthosulfamuron; I+oxadiargyl; I+oxadiazon; I+oxyfluorfen; I+paraquat dichloride; I+pendimethalin; I+penoxsulam; I+phenmedipham; I+picloram; I+picolinafen; I+pinoxaden; I+pretilachlor; I+primisulfuron-methyl; I+prodiamine; I+prometryn; I+propachlor; I+propanil; I+propaquizafop; I+propham; I+propyzamide; I+prosulfocarb; I+prosulfuron; I+pyrasulfotole; I+pyrazolynate, I+pyrazosulfuron-ethyl; I+pyribenzoxim; I+pyridate; I+pyriftalid; I+pyrithiobac-sodium; I+pyroxasulfone; I+pyroxsulam; I+quinclorac; I+quizalofop-P-ethyl; I+rimsulfuron; I+saflufenacil; I+sethoxydim; I+S-metolachlor; I+sulcotrione; I+sulfentrazone; I+tebuthiuron; I+tefuryltrione; I+tembotrione; I+terbuthylazine; I+terbutryn; I+thiencarbazone; I+thifensulfuron; I+tiafenacil; I+tolpyralate; I+topramezone; I+tralkoxydim; I+triafamone; I+triasulfuron; I+tribenuron-methyl; I+triclopyr; I+trifloxysulfuron-sodium; I+trifludimoxazin and tritosulfuron.

Especially preferred examples of such mixtures include:—I+ametryn; I+atrazine; I+bicyclopyrone; I+butafenacil; I+chlorotoluron; I+clodinafop-propargyl; I+clomazone; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+dimethachlor; I+diquat dibromide; I+fluazifop-P-butyl; I+flumetralin; I+fomesafen; I+glufosinate-ammonium; I+glyphosate (including the diammonium, isopropylammonium and potassium salts thereof); I+mesotrione; I+molinate; I+napropamide; I+nicosulfuron; I+paraquat dichloride; I+pinoxaden; I+pretilachlor; I+primisulfuron-methyl; I+prometryn; I+prosulfocarb; I+prosulfuron; I+pyridate; I+pyriftalid; I+pyrazolynate, I+S-metolachlor; I+terbuthylazine; I+terbutryn; I+tralkoxydim; I+triasulfuron and I+trifloxysulfuron-sodium.

Preferred herbicide mixture products for weed control in cereals (especially wheat and/or barley) include:—I+amidosulfuron; I+aminopyralid; I+bromoxynil; I+carfentrazone-ethyl; I+chlorotoluron; I+clodinafop-propargyl; I+clopyralid; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+difenzoquat; I+diflufenican; I+fenoxaprop-P-ethyl; I+florasulam; I+flucarbazone-sodium; I+flufenacet; flupyrsulfuron-methyl-sodium; I+fluroxypyr-meptyl; I+halauxifen-methyl; I+iodosulfuron-methyl-sodium; I+iofensulfuron; I+iofensulfuron-sodium; I+mesosulfuron; I+mesosulfuron-methyl; I+metsulfuron; I+pendimethalin; I+pinoxaden; I+prosulfocarb; I+pyrasulfotole; I+pyroxasulfone; I+pyroxsulam; I+topramezone; I+tralkoxydim; I+triasulfuron and I+tribenuron-methyl.

Preferred herbicide mixture products for weed control in corn include:—I+acetochlor; I+alachlor; I+atrazine; I+bicyclopyrone; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+diflufenzopyr; I+dimethenamid-P; I+flumioxazin; I+fluthiacet-methyl; I+foramsulfuron; I+glufosinate (including the ammonium salt thereof); I+glyphosate (including the diammonium, isopropylammonium and potassium salts thereof); I+isoxaflutole; I+mesotrione; I+nicosulfuron; I+primisulfuron-methyl; I+prosulfuron; I+pyroxasulfone; I+rimsulfuron; I+S-metolachlor, I+terbutylazine; I+tembotrione; I+thiencarbazone and I+thifensulfuron.

Preferred herbicide mixture products for weed control in rice include:—I+2,4-D; I+2,4-D choline salt; I+2,4-D-2-ethylhexyl ester; I+bensulfuron-methyl; I+bispyribac-sodium; I+cafenstrole; I+cinosulfuron; I+clomazone; I+cyhalofop-butyl; I+daimuron; I+dicamba (including the aluminum, aminopropyl, bis-aminopropylmethyl, choline, diglycolamine, dimethylamine, dimethylammonium, potassium and sodium salts thereof); I+esprocarb; I+fenoxaprop-P-ethyl; I+florasulam; I+halauxifen-methyl; I+halosulfuron-methyl; I+iofensulfuron; I+ipfencarbazone; I+mefenacet; I+mesotrione; I+metsulfuron; I+molinate; I+orthosulfamuron; I+oxadiargyl; I+oxadiazon; I+pendimethalin; I+penoxsulam; I+pretilachlor; I+pyrazolynate, I+pyrazosulfuron-ethyl; I+pyribenzoxim; I+pyriftalid; I+quinclorac; I+tefuryltrione; I+triafamone and I+triasulfuron.

Preferred herbicide mixtures for weed control in soybean include:—I+acifluorfen-sodium; I+ametryn; I+atrazine; I+bentazone; I+bicyclopyrone; I+bromoxynil; I+carfentrazone-ethyl; I+chlorimuron-ethyl; I+clethodim; I+clomazone; I+2,4-D (including the choline salt and 2-ethylhexyl ester thereof); I+dicamba (including the aluminum, aminopropyl, bis-aminopropylmethyl, choline, diglycolamine, dimethylamine, dimethylammonium, potassium and sodium salts thereof); I+diquat dibromide; I+diuron; I+fenoxaprop-P-ethyl; I+fluazifop-P-butyl; I+flufenacet; I+flumioxazin; I+fomesafen; I+glufosinate (including the ammonium salt thereof); I+glyphosate (including the diammonium, isopropylammonium and potassium salts thereof); I+imazethapyr; I+lactofen; I+mesotrione; I+metolachlor; I+metribuzin; I+nicosulfuron; I+oxyfluorfen; I+paraquat dichloride; I+pendimethalin; I+pyroxasulfone; I+quizalofop-P-ethyl; I+saflufenacil; I+sethoxydim; I+S-metolachlor and I+sulfentrazone.

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations (wherein “I” represents a compound of formula (I)) include:—I+benoxacor, I+cloquintocet-mexyl; I+cyprosulfamide; I+dichlormid; I+fenchlorazole-ethyl; I+fenclorim; I+fluxofenim; I+furilazole I+isoxadifen-ethyl; I+mefenpyr-diethyl; I+N-(2-methoxybenzoyl)-4-[(methylaminocarbonyl)amino] benzenesulfonamide and I+oxabetrinil.

Particularly preferred are mixtures of a compound of formula (I) with cyprosulfamide, isoxadifen-ethyl, cloquintocet-mexyl and/or N-(2-methoxybenzoyl)-4-[(methyl-aminocarbonyl)amino]benzenesulfonamide.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

The compounds of formula (I) are also useful for pre-harvest desiccation in crops, for example, but not limited to, potatoes, soybean, sunflowers and cotton. Pre-harvest desiccation is a well-known process used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol | 3% |
| (mol. wt. 200) | |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% | 1% |
| emulsion in water) | |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### List of Abbreviations

Boc=tert-butyloxycarbonyl
br=broad
CDCl₃=chloroform-d
CD₃OD=methanol-d
° C.=degrees Celsius
D₂O=water-d
DCM=dichloromethane
d=doublet
dd=double doublet
dt=double triplet
DMSO=dimethylsulfoxide
EtOAc=ethyl acetate
h=hour(s)
HCl=hydrochloric acid
m=multiplet
M=molar
min=minutes
MHz=mega hertz
mL=millilitre
mp=melting point
ppm=parts per million
q=quartet
quin=quintet
rt=room temperature
s=singlet
t=triplet
THF=tetrahydrofuran

### LC/MS=Liquid Chromatography Mass Spectrometry Preparation Examples

### Example 1: Preparation of 2-[[2-[4-(1-methyl-1,2,4-triazol-3-yl)pyridazin-1-ium-1-yl]acetyl]amino]acetic acid chloride A2

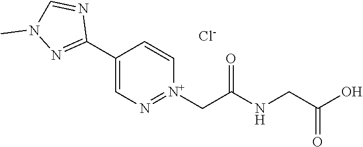 [SMILES: Cn1cnc(-c2cc[n+](CC(=O)NCC(=O)O)nc2)n1 ; [Cl-]]

### Step 1: Preparation of tributyl(pyridazin-4-yl)stannane

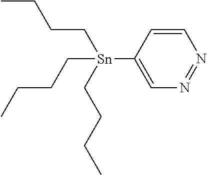 [SMILES: CCC[CH2][Sn]([CH2]CCC)([CH2]CCC)[c]1ccnnc1]

To a solution of lithium diisopropylamide (1M solution in tetrahydrofuran, 125 mL) at −78° C. under nitrogen was added a solution of pyridazine (10 g) and tri-n-butyltin chloride (44.6 g) in tetrahydrofuran (100 mL) drop wise. The reaction mixture was stirred at −78° C. for 1 hour. The reaction mixture was warmed to room temperature and quenched with saturated aqueous ammonium chloride (100 mL) and extracted with ethyl acetate (3×150 mL). The organic layer was dried over sodium sulfate, concentrated and purified by chromatography on silica eluting with 30% ethyl acetate in hexanes to afford tributyl(pyridazin-4-yl)stannane as a pale brown liquid.

¹H NMR (400 MHz, CDCl₃) 9.17 (t, 1H) 9.02 (dd, 1H) 7.54 (dd, 1H) 1.57-1.49 (m, 6H) 1.37-1.29 (m, 6H) 1.19-1.13 (m, 6H) 0.92-0.86 (m, 9H).

### Step 2: Preparation of 4-(1-methyl-1,2,4-triazol-3-yl)pyridazine

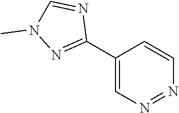

To a solution of 3-bromo-1-methyl-1,2,4-triazole (0.197 g) in 1,4-dioxane (10 mL) was added tributyl(pyridazin-4-yl)stannane (0.5 g), cesium fluoride (0.185 g), tetrakis(triphenylphosphine) palladium(0) (0.376 g), cuprous iodide (0.011 g) and lithium chloride (0.826 g). The reaction mixture was purged with argon for 20 minutes then heated at 110° C. for 16 hours. After cooling to room temperature the mixture was filtered through Celite and washed with 10% methanol in dichloromethane (2×20 mL). The filtrate was concentrated and purified by silica gel chromatography eluting with 5% methanol in dichloromethane to afford 4-(1-methyl-1,2,4-triazol-3-yl)pyridazine as a light brown solid.

¹H NMR (400 MHz, CDCl₃) 9.86 (dd, 1H), 9.29 (dd, 1H), 8.17 (s, 1H), 8.10 (dd, 1H), 4.04 (s, 3H)

### Step 3: Preparation of methyl 2-[(2-bromoacetyl)amino]acetate

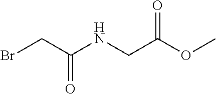

A mixture of methyl 2-aminoacetate hydrochloride (10 g) in dichloromethane (200 mL), under nitrogen atmosphere, was cooled to −78° C. and triethylamine (22.2 mL) was added, followed by 2-bromoacetyl bromide (7.66 mL). The resulting reaction mixture was allowed to warm to room temperature and stirred for 16 hours. Water (20 mL) was added to reaction mixture and this was extracted with dichloromethane (3×200 mL). The combined organic layers were washed with brine (200 mL), dried over sodium sulfate, concentrated and purified by silica gel chromatography eluting with a mixture of ethyl acetate in hexanes to give methyl 2-[(2-bromoacetyl)amino]acetate as a brown liquid.

¹H NMR (400 MHz, DMSO-d₆) 8.70 (brs, 1H), 4.94 (s, 2H), 4.88 (d, 2H), 3.62 (s, 3H)

### Step 4: Preparation of methyl 2-[[2-[4-(1-methyl-1,2,4-triazol-3-yl)pyridazin-1-ium-1-yl]acetyl]amino]acetate 2,2,2-trifluoroacetate A4

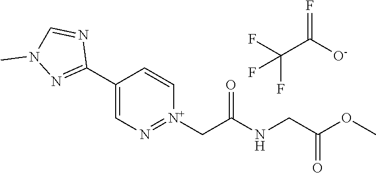

To a stirred solution of 4-(1-methyl-1,2,4-triazol-3-yl)pyridazine (0.4 g) in acetonitrile (10 mL) was added methyl 2-[(2-bromoacetyl)amino]acetate (0.743 g) at room temperature. The resulting reaction mixture was heated at 70° C. for 16 hours. The reaction mixture was cooled to room temperature and concentrated. The resulting residue was dissolved in water (20 mL) and washed with 10% methanol in dichloromethane (2×20 mL). The aqueous layer was concentrated and purified by reverse phase chromatography eluting with 0.05% trifluoroacetic acid in a water and acetonitrile mix to afford methyl 2-[[2-[4-(1-methyl-1,2,4-triazol-3-yl)pyridazin-1-ium-1-yl]acetyl]amino]acetate 2,2,2-trifluoroacetate as a brown solid.

¹H NMR (400 MHz, D₂O) 10.01 (d, 1H), 9.78 (d, 1H), 9.06-9.01 (m, 1H), 8.68 (s, 1H), 5.84 (s, 2H), 4.16 (s, 2H), 4.11 (s, 3H), 3.77 (s, 3H) (NH proton missing)

### Step 5: Preparation of 2-[[2-[4-(1-methyl-1,2,4-triazol-3-yl)pyridazin-1-ium-1-yl]acetyl]amino]acetic acid chloride A2

To a mixture of methyl 2-[[2-[4-(1-methyl-1,2,4-triazol-3-yl)pyridazin-1-ium-1-yl]acetyl]amino]acetate 2,2,2-trifluoroacetate (0.14 g) in water (2 mL) was added conc. hydrochloric acid (2 mL). The resulting reaction mixture was heated at 70° C. for 2 hours. The reaction mixture was cooled, diluted with water (25 mL) and washed with dichloromethane (2×25 mL). The aqueous layer was concentrated to afford 2-[[2-[4-(1-methyl-1,2,4-triazol-3-yl)pyridazin-1-ium-1-yl]acetyl]amino]acetic acid chloride as a light brown solid.

¹H NMR (400 MHz, D₂O) 9.90 (s, 1H), 9.66 (d, 1H), 8.94-8.88 (m, 1H), 8.58 (s, 1H), 6.04-6.01 (m, 0.25H), 5.61 (s, 1.75H), 4.04-3.99 (m, 3H), 3.84 (s, 2H), 2.69 (s, 3H) (CO₂H proton missing)

### Example 2: Preparation of 2-[[2-[4-(1-methyl-1,2,4-triazol-3-yl)pyridazin-1-ium-1-yl]acetyl]amino]ethanesulfonate A5

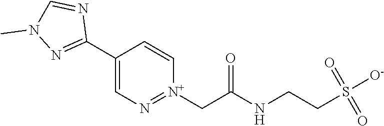 [SMILES: Cn1cnc(-c2cc[n+](CC(=O)NCCS(=O)(=O)[O-])nc2)n1]

### Step 1: Preparation of 2,2-dimethylpropyl 2-aminoethanesulfonate

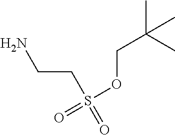

A mixture of ammonium hydroxide (13 mL) and tetrahydrofuran (20 mL) was cooled to ˜0° C. and a solution of 2,2-dimethylpropyl ethenesulfonate (4 g) in tetrahydrofuran (20 mL) was added drop wise. The mixture was stirred at ˜0° C. for 1 hour and then at room temperature for 16 hours.

The mixture was partitioned between water (50 mL) and ethyl acetate (100 mL). The aqueous layer was extracted with further ethyl acetate (2×100 mL). The combined organic layers were dried over sodium sulfate, concentrated and purified by chromatography on silica eluting with a mixture of ethyl acetate in cyclohexane to give 2,2-dimethylpropyl 2-aminoethanesulfonate as a pale-yellow liquid.

¹H NMR (400 MHz, DMSO-d₆) 3.86 (s, 2H), 3.34-3.38 (m, 2H), 2.91 (t, 2H), 0.93 ppm (s, 9H)

### Step 2: Preparation of 2,2-dimethylpropyl 2-[(2-bromoacetyl)amino]ethanesulfonate

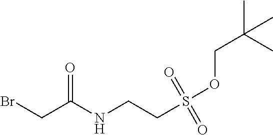 [SMILES: CC(C)(C)COS(=O)(=O)CCNC(=O)CBr]

A mixture of 2,2-dimethylpropyl 2-aminoethanesulfonate (1 g) in dichloromethane (10 mL), under nitrogen atmosphere, was cooled to −10° C. and triethylamine (1.02 mL) was added, followed by a solution of 2-bromoacetyl bromide (0.468 mL) in dichloromethane (5 mL). The resulting reaction mixture was stirred at −10° C. for 30 minutes then allowed to warm to room temperature and stirred for 4 hours. Water (50 mL) was added to reaction mixture and this was extracted with dichloromethane (2×75 mL). The combined organic layers were washed with brine (200 mL), dried over sodium sulfate, concentrated and purified by silica gel chromatography eluting with a mixture of ethyl acetate in hexanes to give 2,2-dimethylpropyl 2-[(2-bromoacetyl)amino]ethanesulfonate as a brown liquid.

¹H NMR (400 MHz, CDCl₃) 7.12 (br s, 1H), 3.92 (s, 2H), 3.88 (s, 2H), 3.84-3.77 (m, 2H), 3.36-3.31 (m, 2H), 1.00 (s, 9H)

### Step 3: Preparation of 2,2-dimethylpropyl 2-[[2-[4-(1-methyl-1,2,4-triazol-3-yl)pyridazin-1-ium-1-yl]acetyl]amino]ethanesulfonate bromide

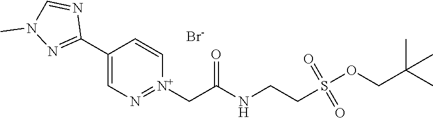 [SMILES: Cn1cnc(-c2cc[n+](CC(=O)NCCS(=O)(=O)OCC(C)(C)C)nc2)n1 ; [Br-]]

To a stirred solution of 4-(1-methyl-1,2,4-triazol-3-yl)pyridazine (0.4 g) in acetonitrile (10 mL) was added 2,2-dimethylpropyl 2-[(2-bromoacetyl)amino]ethanesulfonate (0.743 g) at room temperature. The resulting reaction mixture was heated at 70° C. for 16 hours. The reaction mixture was diluted with water (50 mL) and washed with 10% methanol in dichloromethane (2×25 mL). The aqueous layer was concentrated to give a crude mixture of 2,2-dimethylpropyl 2-[[2-[4-(1-methyl-1,2,4-triazol-3-yl)pyridazin-1-ium-1-yl]acetyl]amino]ethanesulfonate bromide and 2,2-dimethylpropyl 2-[[2-[5-(1-methyl-1,2,4-triazol-3-yl)pyridazin-1-ium-1-yl]acetyl]amino]ethanesulfonate bromide, which was used without further purification.

### Step 4: Preparation of 2-[[2-[4-(1-methyl-1,2,4-triazol-3-yl)pyridazin-1-ium-1-yl]acetyl]amino]ethanesulfonate A5

To a solution of crude 2,2-dimethylpropyl 2-[[2-[4-(1-methyl-1,2,4-triazol-3-yl)pyridazin-1-ium-1-yl]acetyl]amino]ethanesulfonate bromide (0.2 g) in water (2.5 mL) was added concentrated hydrochloric acid (2.5 mL) at room temperature. The reaction mixture was heated at 60° C. for 2 hours. The reaction mixture was diluted with water (25 mL) and washed with dichloromethane (2×25 mL). The aqueous layer was concentrated and purified by preparative reverse phase HPLC to give 2-[[2-[4-(1-methyl-1,2,4-triazol-3-yl)pyridazin-1-ium-1-yl]acetyl]amino]ethanesulfonate as a brown solid.

¹H NMR (400 MHz, D₂O) 9.91-9.87 (m, 1H), 9.68-9.64 (m, 1H), 8.94-8.89 (m, 1H), 8.57 (s, 1H), 5.64 (s, 2H), 4.01 (s, 3H), 3.63-3.57 (m, 2H), 3.04 (s, 2H) (NH proton missing)

### Example 3: Preparation of 2-[[4-(1-methyl-1,2,4-triazol-3-yl)pyridazin-1-ium-1-yl]methylsulfanyl]acetic acid chloride A8

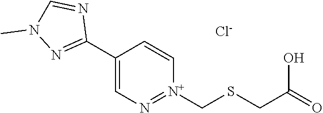 [SMILES: Cn1cnc(-c2cc[n+](CSCC(=O)O)nc2)n1 ; [Cl-]]

### Step 1: Preparation of ethyl 2-(chloromethylsulfanyl)acetate

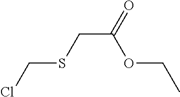

Sodium hydride (60% in mineral oil, 0.72 g) was washed with cyclohexane (×2) then was suspended in dry tetrahydrofuran (10 mL), under nitrogen atmosphere. To this was added a solution of ethyl thioglycolate (2.163 g) in dry tetrahydrofuran (2.6 mL) drop wise over 40 minutes at room temperature. After stirring for 30 minutes this suspension was added drop wise to cooled (˜0° C.) bromochloromethane (5.9 mL) over 40 minutes. The mixture was stirred at ˜0° C. for 18 hours. The mixture was diluted with pentane (5 mL) and filtered through celite, washing through with further pentane (5 mL). The filtrate was cautiously concentrated to give crude ethyl 2-(chloromethylsulfanyl)acetate, which was used without further purification.

¹H NMR (400 MHz, CDCl₃) 4.84 (s, 2H), 4.22 (q, 2H), 3.47 (s, 2H), 1.30 (t, 3H)

### Step 2: Preparation of ethyl 2-[[4-(1-methyl-1,2,4-triazol-3-yl)pyridazin-1-ium-1-yl]methylsulfanyl]acetate chloride

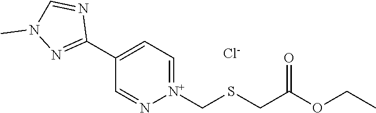 [SMILES: CCOC(=O)CSC[n+]1ccc(-c2ncn(C)n2)cn1 ; [Cl-]]

To a stirred solution of 4-(1-methyl-1,2,4-triazol-3-yl)pyridazine (0.1 g) in acetonitrile (15 mL) was added ethyl 2-(chloromethylsulfanyl)acetate (0.157 g) at room temperature. The resulting reaction mixture was heated at 70° C. for 24 hours. The reaction mixture was diluted with water (20 mL) and washed with 10% methanol in dichloromethane (2×20 mL). The aqueous layer was concentrated and purified by preparative reverse phase HPLC (100% water) to give ethyl 2-[[4-(1-methyl-1,2,4-triazol-3-yl)pyridazin-1-ium-1-yl]methylsulfanyl]acetate chloride as a dark brown liquid.

¹H NMR (400 MHz, D₂O) 9.88-9.94 (m, 2H), 8.90-8.96 (m, 1H), 8.60 (s, 1H), 6.00 (s, 2H), 3.98-4.08 (m, 5H), 3.68 (s, 2H), 1.10-1.18 (m, 3H)

### Step 3: Preparation of 2-[[4-(1-methyl-1,2,4-triazol-3-yl)pyridazin-1-ium-1-yl]methylsulfanyl]acetic acid chloride A8

A mixture of ethyl 2-[[4-(1-methyl-1,2,4-triazol-3-yl)pyridazin-1-ium-1-yl]methylsulfanyl]acetate chloride (0.085 g) and 6M aqueous hydrochloric acid (5 mL) was stirred at room temperature for 16 hours. The reaction mixture was concentrated, diluted with water (10 mL) and washed with dichloromethane (2×10 mL). The aqueous layer was concentrated to give 2-[[4-(1-methyl-1,2,4-triazol-3-yl)pyridazin-1-ium-1-yl]methylsulfanyl]acetic acid chloride as a light brown solid.

¹H NMR (400 MHz, D₂O) 9.89-9.83 (m, 2H), 8.91-8.85 (m, 1H), 8.57 (s, 1H), 5.96 (s, 2H), 4.01 (s, 3H), 3.63 (s, 2H) (CO₂H proton missing)

### Example 4: Preparation of 2-[2-(4-thiazol-2-ylpyridazin-1-ium-1-yl)ethylsulfonylamino]acetic acid 2,2,2-trifluoroacetate A17

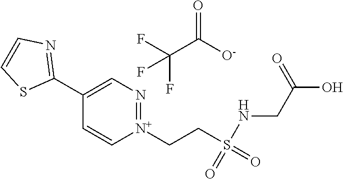 [SMILES: O=C(O)CNS(=O)(=O)CC[n+]1ccc(-c2nccs2)cn1 ; O=C([O-])C(F)(F)F]

### Step 1: Preparation of 2-pyridazin-4-ylthiazole

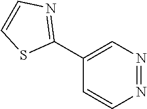 [SMILES: c1cc(-c2nccs2)cnn1]

To a solution of 2-bromothiazole (2 g) in 1,4-dioxane (50 mL) was added tributyl(pyridazin-4-yl)stannane (5.69 g), cesium fluoride (1.85 g), tetrakis(triphenylphosphine) palladium(0) (1.41 g) and cuprous iodide (0.116 g). The reaction mixture was purged with argon for 20 minutes then heated at 110° C. for 16 hours. After cooling to room temperature, the mixture was filtered through Celite and washed with 10% methanol in dichloromethane (2×20 mL). The filtrate was concentrated and purified by silica gel chromatography eluting with a mixture of methanol in dichloromethane to give 2-pyridazin-4-ylthiazole as a brown solid.

¹H NMR (400 MHz, CDCl₃) 9.75 (dd, 1H), 9.31 (dd, 1H), 8.06 (d, 1H), 7.96 (dd, 1H), 7.60 (d, 1H)

### Step 2: Preparation of tert-butyl 2-(vinylsulfonylamino)acetate

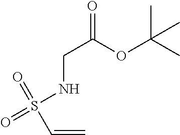

A solution of tert-butyl 2-aminoacetate (3 g) and triethylamine (3.51 mL) in dichloromethane (100 mL) was cooled to −10° C. and a solution of 2-chloroethanesulfonyl chloride (2.5 mL) in dichloromethane (4 mL) was added over a period of 15 minutes. The resulting mixture was stirred at ˜0° C. for 8 hours and then at room temperature overnight. The product mixture was concentrated and the residue was purified by chromatography on silica eluting with a mixture of ethyl acetate in iso-hexanes to give tert-butyl 2-(vinylsulfonylamino)acetate as a white solid.

¹H NMR (400 MHz, CDCl₃) 6.56 (dd, 1H), 6.26 (d, 1H), 5.94 (d, 1H), 3.73 (d, 2H), 1.47 (s, 9H)

### Step 3: Preparation of 2-[2-(4-thiazol-2-ylpyridazin-1-ium-1-yl)ethylsulfonylamino]acetic acid 2,2,2-trifluoroacetate A17

A mixture of 2-pyridazin-4-ylthiazole (0.2 g), tert-butyl 2-(vinylsulfonylamino)acetate (0.491 g), trifluoroacetic acid (5 mL) and water (5 mL) was heated at 120° C. for 48 hours. The reaction mixture was concentrated and purified by preparative reverse phase HPLC (trifluoroacetic acid was present in the eluent) to give 2-[2-(4-thiazol-2-ylpyridazin-1-ium-1-yl)ethylsulfonylamino]acetic acid 2,2,2-trifluoroacetate as a brown solid.

¹H NMR (400 MHz, D₂O) 9.91 (s, 1H), 9.71 (br d, 1H), 8.84 (br d, 1H), 8.22-8.16 (m, 1H), 8.12-8.07 (m, 1H), 5.26 (br t, 2H), 4.03 (br t, 2H), 3.90 (s, 2H) (NH and CO₂H protons missing)

### Example 5: Preparation of 2-pyridazin-4-yloxazole

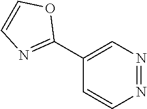 [SMILES: c1cc(-c2ncco2)cnn1]

To a solution of 4-bromopyridazine (3 g) in 1,4-dioxane (50 mL) was added tributyl(oxazol-2-yl)stannane (7.06 g), cesium fluoride (2.72 g), tetrakis(triphenylphosphine) palladium(0) (2.07 g) and cuprous iodide (0.171 g). The reaction mixture was purged with argon for 20 minutes then heated at 140° C. for 1 hour in a sealed tube. After cooling to room temperature, the mixture was filtered through Celite and washed with 10% methanol in dichloromethane (2×50 mL). The filtrate was concentrated and purified by silica gel chromatography eluting with a mixture of ethyl acetate in hexanes to give 2-pyridazin-4-yloxazole as a light brown solid.

¹H NMR (400 MHz, CDCl₃) 9.80 (s, 1H), 9.34-9.40 (m, 1H), 7.98-8.04 (m, 1H), 7.88 (s, 1H), 7.40 (s, 1H)

### Example 6: Preparation of 3-pyridazin-4-yl-1,2,4-thiadiazole

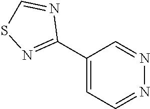 [SMILES: c1cc(-c2ncsn2)cnn1]

### Step 1: Preparation of pyridazine-4-carboxamidine hydrochloride

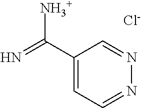

To a mixture of pyridazine-4-carbonitrile (3.5 g) in methanol (18 mL) was added sodium methoxide (25% in methanol, 0.78 mL) at room temperature and the reaction mixture was stirred for 3 hours. To this mixture was added ammonium chloride (2 g) and stirring was continued at room temperature for a further 18 hours. The reaction mixture was concentrated and the resulting residue was washed with tert-butyl methyl ether to give pyridazine-4-carboxamidine hydrochloride as a brown solid.

¹H NMR (400 MHz, DMSO-d₆) 9.58-9.60 (m, 2H), 8.12-8.14 (m, 1H)

### Step 2: Preparation of 3-pyridazin-4-yl-4H-1,2,4-thiadiazole-5-thione

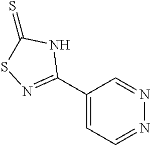 [SMILES: S=c1[nH]c(-c2ccnnc2)ns1]

To a mixture of pyridazine-4-carboxamidine hydrochloride (0.05 g), carbon disulfide (0.5M in tetrahydrofuran, 2 mL), sulfur (0.013 g) and methanol (0.5 mL) was added sodium methoxide (25% in methanol, 0.144 mL) and the reaction was heated at 60° C. for 5 hours. The reaction mixture was concentrated and purified by silica gel chromatography eluting with ethyl acetate in methanol to give 3-pyridazin-4-yl-4H-1,2,4-thiadiazole-5-thione as dark brown solid.

¹H NMR (400 MHz, DMSO-d₆) 9.73 (s, 1H) 9.33 (dd, 1H) 8.12 (dd, 1H)

### Step 3: Preparation of 3-pyridazin-4-yl-1,2,4-thiadiazole

A mixture of 3-pyridazin-4-yl-4H-1,2,4-thiadiazole-5-thione (0.5 g) and acetic acid (12.74 mL) was cooled to 15° C. and hydrogen peroxide (1.56 mL) was added drop wise. The mixture was stirred at room temperature for 3 hours, when further hydrogen peroxide (1.56 mL) was added. After a further 2 hours stirring the reaction mixture was quenched with sodium metabisulfite solution, neutralised and extracted with ethyl acetate (3×50 mL). The combined organic phases were dried over anhydrous sodium sulfate and purified by silica gel chromatography eluting with ethyl acetate to give 3-pyridazin-4-yl-1,2,4-thiadiazole.

¹H NMR (400 MHz, CDCl₃) 10.09 (dd, 1H), 10.01 (s, 1H), 9.41 (dd, 1H), 8.33 (dd, 1H)

### Example 7: Preparation of 2,2-dimethylpropyl 2-[(2-bromoacetyl)amino]ethanesulfonate

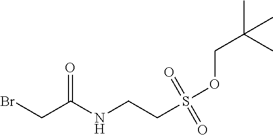 [SMILES: CC(C)(C)COS(=O)(=O)CCNC(=O)CBr]

### Step 1: Preparation of 2,2-dimethylpropyl 2-aminoethanesulfonate

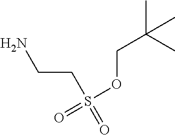

A mixture of ammonium hydroxide (13 mL) and tetrahydrofuran (20 mL) was cooled to ˜0° C. and a solution of 2,2-dimethylpropyl ethenesulfonate (4 g) in tetrahydrofuran (20 mL) was added drop wise. The mixture was stirred at ˜0° C. for 1 hour and then at room temperature for 16 hours.

The mixture was partitioned between water (50 mL) and ethyl acetate (100 mL). The aqueous layer was extracted with further ethyl acetate (2×100 mL). The combined organic layers were dried over sodium sulfate, concentrated and purified by chromatography on silica eluting with a mixture of ethyl acetate in cyclohexane to give 2,2-dimethylpropyl 2-aminoethanesulfonate as a pale-yellow liquid.

¹H NMR (400 MHz, DMSO-d₆) 3.86 (s, 2H), 3.34-3.38 (m, 2H), 2.91 (t, 2H), 0.93 ppm (s, 9H)

### Step 2: Preparation of 2,2-dimethylpropyl 2-[(2-bromoacetyl)amino]ethanesulfonate

A mixture of 2,2-dimethylpropyl 2-aminoethanesulfonate (1 g) in dichloromethane (10 mL), under nitrogen atmosphere, was cooled to −10° C. and triethylamine (1.02 mL) was added, followed by a solution of 2-bromoacetyl bromide (0.468 mL) in dichloromethane (5 mL). The resulting reaction mixture was stirred at −10° C. for 30 minutes then allowed to warm to room temperature and stirred for 4 hours. Water (50 mL) was added to reaction mixture and this was extracted with dichloromethane (2×75 mL). The combined organic layers were washed with brine (200 mL), dried over sodium sulfate, concentrated and purified by silica gel chromatography eluting with a mixture of ethyl acetate in hexanes to give 2,2-dimethylpropyl 2-[(2-bromoacetyl)amino]ethanesulfonate as a brown liquid.

¹H NMR (400 MHz, CDCl₃) 7.12 (br s, 1H), 3.92 (s, 2H), 3.88 (s, 2H), 3.84-3.77 (m, 2H), 3.36-3.31 (m, 2H), 1.00 (s, 9H)

### Example 8: Preparation of methyl 3-(chloromethylsulfanyl)propanoate

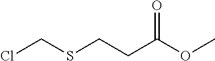

Sodium hydride (60% in mineral oil, 0.36 g) was washed with cyclohexane (×2) then was suspended in dry tetrahydrofuran (5 mL), under nitrogen atmosphere. To this was added a solution of methyl 3-mercaptopropionate (1 mL) in dry tetrahydrofuran (1.3 mL) drop wise over 40 minutes at room temperature. After stirring for 30 minutes this suspension was added drop wise to cooled (˜0° C.) bromochloromethane (2.92 mL) over 40 minutes. The mixture was stirred at ˜0° C. for 18 hours. The mixture was diluted with tert-butyl methyl ether (5 mL) and filtered through celite, washing through with further tert-butyl methyl ether (5 mL). The filtrate was cautiously concentrated to give crude methyl 3-(chloromethylsulfanyl)propanoate, which was used without further purification.

¹H NMR (400 MHz, CDCl₃) 4.75 (s, 2H), 3.72 (s, 3H), 3.03 (t, 2H), 2.73 (t, 2H)

Additional compounds in Table A (below) were prepared by analogous procedures, from appropriate starting materials. The skilled person would understand that the compounds of formula (I) may exist as an agronomically acceptable salt, a zwitterion or an agronomically acceptable salt of a zwitterion as described hereinbefore. Where mentioned the specific counterion is not considered to be limiting, and the compound of formula (I) may be formed with any suitable counter ion.

NMR spectra contained herein were recorded on either a 400 MHz Bruker AVANCE III HD equipped with a Bruker SMART probe unless otherwise stated. Chemical shifts are expressed as ppm downfield from TMS, with an internal reference of either TMS or the residual solvent signals. The following multiplicities are used to describe the peaks: s=singlet, d=doublet, t=triplet, dd=double doublet, dt=double triplet, q=quartet, quin=quintet, m=multiplet. Additionally br. is used to describe a broad signal and app. is used to describe and apparent multiplicity.

TABLE A
Physical Data for Compounds of the Invention

| Compound | | |
|---|---|---|
| Number | Structure | ¹H NMR |
| A1 | 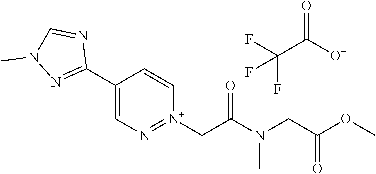 | (400 MHz, D₂O) 9.91 (d, 1H), 9.62 (d, 1H), 8.93 (dd, 1H), 8.58 (s, 1H), 6.03 (s, 2H), 4.19 (s, 2H), 4.02 (s, 3H), 3.68 (s, 3H), 3.17 (s, 3H) |
| A2 | 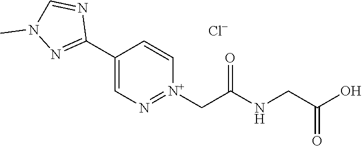 [SMILES: Cn1cnc(-c2cc[n+](CC(=O)NCC(=O)O)nc2)n1 ; [Cl-]] | (400 MHz, D₂O) 9.89 (s, 1H), 9.66 (d, 1H), 8.91 (dd, 1H), 8.58 (s, 1H), 5.64 (s, 2H), 4.01 (s, 3H), 3.79 (s, 2H) (NH and CO₂H protons missing) |
| A3 | 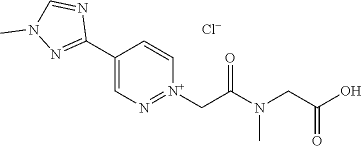 [SMILES: CN(CC(=O)O)C(=O)C[n+]1ccc(-c2ncn(C)n2)cn1 ; [Cl-]] | (400 MHz, D₂O) 9.90 (s, 1H), 9.66 (d, 1H), 8.94- 8.88 (m, 1H), 8.58 (s, 1H), 6.04-6.01 (m, 0.25H), 5.61 (s, 1.75H), 4.04-3.99 (m, 3H), 3.84 (s, 2H), 2.69 (s, 3H) (CO₂H proton missing) |
| A4 | 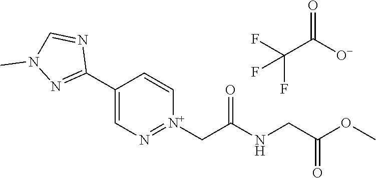 | (400 MHz, D₂O) 10.01 (d, 1H), 9.78 (d, 1H), 9.06-9.01 (m, 1H), 8.68 (s, 1H), 5.84 (s, 2H), 4.16 (s, 2H), 4.11 (s, 3H), 3.77 (s, 3H) (NH proton missing) |
| A5 | 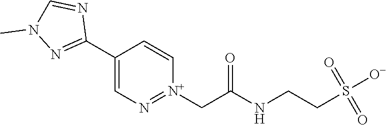 [SMILES: Cn1cnc(-c2cc[n+](CC(=O)NCCS(=O)(=O)[O-])nc2)n1] | (400 MHz, D₂O) 9.91-9.87 (m, 1H), 9.68-9.64 (m, 1H), 8.94-8.89 (m, 1H), 8.57 (s, 1H), 5.64 (s, 2H), 4.01 (s, 3H), 3.63-3.57 (m, 2H), 3.04 (s, 2H) (NH proton missing) |
| A6 | 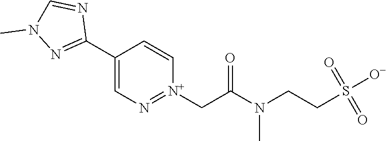 [SMILES: CN(CCS(=O)(=O)[O-])C(=O)C[n+]1ccc(-c2ncn(C)n2)cn1] | (400 MHz, D₂O) 9.88 (s, 1H), 9.58 (br d, 1H), 8.93-8.87 (m, 1H), 8.56 (s, 1H), 6.07 (s, 0.8H), 5.89 (s, 1.2H), 4.00 (s, 3H), 3.84-3.78 (m, 0.8H), 3.74-3.67 (m, 1.2H), 3.22-3.14 (m, 0.8H), 3.12 (s, 1.8H), 3.10-3.04 (m, 1.2H), 2.91 (s, 1.2H) |
| A7 | 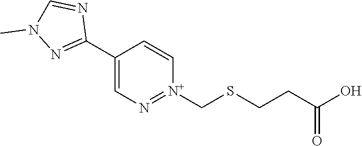 [SMILES: Cn1cnc(-c2cc[n+](CSCCC(=O)O)nc2)n1] | (400 MHz, D₂O) 9.91-9.85 (m, 2H), 8.92-8.87 (m, 1H), 8.58 (s, 1H), 5.92 (s, 2H), 4.02 (s, 3H), 2.96 (t, 2H), 2.68 (t, 2H) (CO₂H proton missing) |
| A8 | 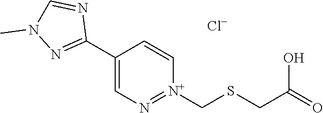 [SMILES: Cn1cnc(-c2cc[n+](CSCC(=O)O)nc2)n1 ; [Cl-]] | (400 MHz, D₂O) 9.89-9.83 (m, 2H), 8.91-8.85 (m, 1H), 8.57 (s, 1H), 5.96 (s, 2H), 4.01 (s, 3H), 3.63 (s, 2H) (CO₂H proton missing) |
| A9 | 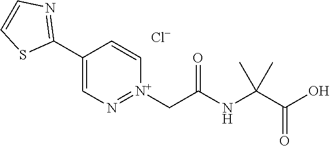 [SMILES: CC(C)(NC(=O)C[n+]1ccc(-c2nccs2)cn1)C(=O)O ; [Cl-]] | (400 MHz, D₂O) 9.84 (s, 1H), 9.56 (d, 1H), 8.85 (br d, 1H), 8.17-8.14 (m, 1H), 8.09-8.05 (m, 1H), 5.58 (s, 2H), 1.39 (s, 6H) (NH and CO₂H protons missing) |
| A10 | 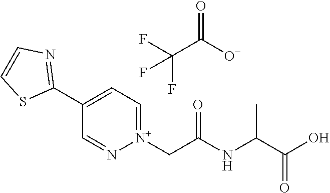 [SMILES: CC(NC(=O)C[n+]1ccc(-c2nccs2)cn1)C(=O)O ; O=C([O-])C(F)(F)F] | (400 MHz, D20) 9.79-9.90 (m, 1H), 9.55-9.65 (m, 1H), 8.82-8.90 (m, 1H), 8.13-8.20 (m, 1H), 8.04-8.11 (m, 1H), 5.60-5.70 (m, 2H), 4.29-4.41 (m, 1H), 1.33-1.41 (m, 3H) (NH and CO₂H protons missing) |
| A11 | 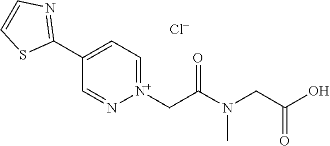 [SMILES: CN(CC(=O)O)C(=O)C[n+]1ccc(-c2nccs2)cn1 ; [Cl-]] | (400 MHz, D₂O) 9.87 (d, 1H), 9.56 (d, 1H), 8.87 (dd, 1H), 8.17 (d, 1H), 8.09 (d, 1H), 5.99-5.94 (m, 2H), 4.13 (s, 2H), 3.13 (s, 3H) (CO₂H proton missing) |
| A12 | 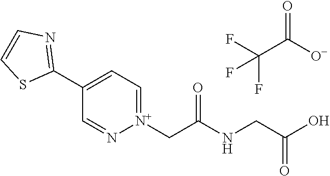 [SMILES: O=C(O)CNC(=O)C[n+]1ccc(-c2nccs2)cn1 ; O=C([O-])C(F)(F)F] | (400 MHz, D₂O) 9.89 (d, 1H), 9.64 (d, 1H), 8.90 (dd, 1H), 8.20 (d,1H), 8.11 (d, 1H), 5.71 (s, 2H), 4.02 (s, 2H) (NH and CO₂H protons missing) |
| A13 | 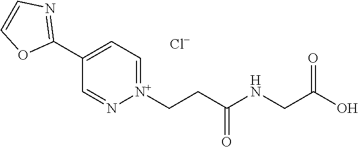 [SMILES: O=C(O)CNC(=O)CC[n+]1ccc(-c2ncco2)cn1 ; [Cl-]] | (400 MHz, D₂O) 9.84 (d, 1H), 9.71 (d, 1H), 8.83 (dd, 1H), 8.19 (s, 1H), 7.55 (s, 1H), 5.15- 5.01 (m, 2H), 3.82 (s, 2H), 3.21-3.10 (m, 2H) (NH and CO₂H protons missing) |
| A14 | 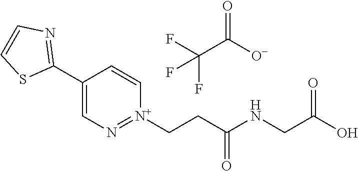 [SMILES: O=C(O)CNC(=O)CC[n+]1ccc(-c2nccs2)cn1 ; O=C([O-])C(F)(F)F] | (400 MHz, D₂O) 9.87 (s, 1H), 9.62 (br d, 1H), 8.86-8.78 (m, 1H), 8.21-8.16 (m, 1H), 8.11- 8.06 (m, 1H), 5.09-5.01 (m, 2H), 3.85 (s, 2H), 3.19-3.11 (m, 2H) (NH and CO₂H protons missing) |
| A15 | 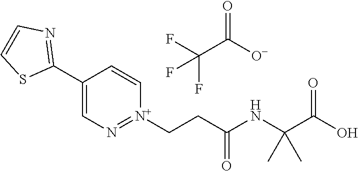 [SMILES: CC(C)(NC(=O)CC[n+]1ccc(-c2nccs2)cn1)C(=O)O ; O=C([O-])C(F)(F)F] | (400 MHz, D₂O) 9.92-9.84 (m, 1H), 9.59 (d, 1H), 8.84-8.78 (m, 1H), 8.18 (d, 1H), 8.08 (d, 1H), 5.01 (br t, 2H), 3.05 (t, 2H), 1.30 (s, 6H) (NH and CO₂H protons missing) |
| A16 | 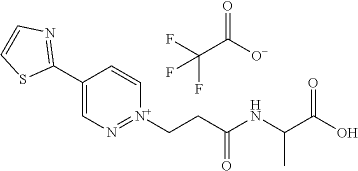 [SMILES: CC(NC(=O)CC[n+]1ccc(-c2nccs2)cn1)C(=O)O ; O=C([O-])C(F)(F)F] | (400 MHz, D20) 9.94-9.90 (m, 1H), 9.65 (d, 1H), 8.89-8.82 (m, 1H), 8.23 (d, 1H), 8.13 (d, 1H), 5.08 (br t, 2H), 4.29-4.20 (m, 1H), 3.19- 3.12 (m, 2H), 1.31 (d, 3H) (NH and CO₂H protons missing) |
| A17 | 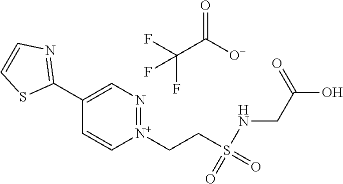 [SMILES: O=C(O)CNS(=O)(=O)CC[n+]1ccc(-c2nccs2)cn1 ; O=C([O-])C(F)(F)F] | (400 MHz, D₂O) 9.91 (s, 1H), 9.71 (br d, 1H), 8.84 (br d, 1H), 8.22-8.16 (m, 1H), 8.12-8.07 (m, 1H), 5.26 (br t, 2H), 4.03 (br t, 2H), 3.90 (s, 2H) (NH and CO₂H protons missing) |
| A18 | 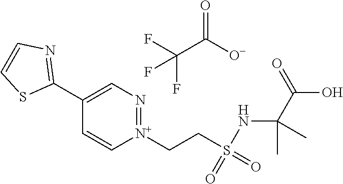 [SMILES: CC(C)(NS(=O)(=O)CC[n+]1ccc(-c2nccs2)cn1)C(=O)O ; O=C([O-])C(F)(F)F] | (400 MHz, D₂O) 9.82 (d, 1H), 9.60 (d, 1H), 8.74 (dd, 1H), 8.09 (d, 1H), 8.00 (d, 1H), 5.14 (t, 2H), 3.92 (t, 2H), 1.30 (s, 6H) (NH and CO₂H protons missing) |
| A19 | 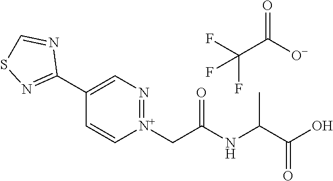 [SMILES: CC(NC(=O)C[n+]1ccc(-c2ncsn2)cn1)C(=O)O ; O=C([O-])C(F)(F)F] | (400 MHz, D₂O) 10.36 (s, 1H), 10.22 (d, 1H), 9.86 (d, 1H), 9.28 (dd, 1H), 5.80 (s, 2H), 4.27- 4.48 (m, 1H), 1.44 (d, 3H) (NH and CO₂H protons missing) |
| A20 | 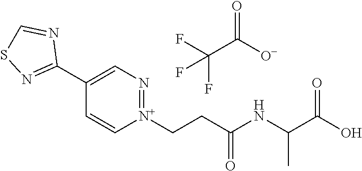 [SMILES: CC(NC(=O)CC[n+]1ccc(-c2ncsn2)cn1)C(=O)O ; O=C([O-])C(F)(F)F] | (400 MHz, D₂O) 10.35 (s, 1H), 10.19 (d, 1H), 9.84 (d, 1H), 9.18 (dd, 1H), 5.13-5.21 (m, 2H), 4.24 (q, 1H), 3.20 (td, 2H), 1.32 (d, 3H) (NH and CO₂H protons missing) |
| A21 | 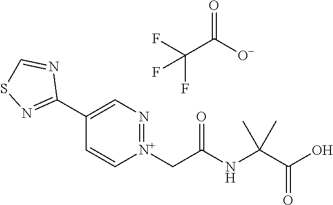 [SMILES: CC(C)(NC(=O)C[n+]1ccc(-c2ncsn2)cn1)C(=O)O ; O=C([O-])C(F)(F)F] | (400 MHz, D₂O) 10.36 (s, 1H), 10.22 (d, 1H), 9.84 (d, 1H), 9.28 (dd, 1H), 5.75 (s, 2H), 1.49 (s, 6H) (NH and CO₂H protons missing) |
| A22 | 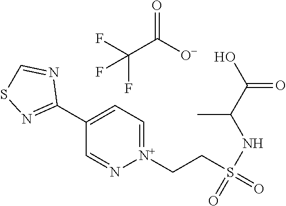 [SMILES: CC(NS(=O)(=O)CC[n+]1ccc(-c2ncsn2)cn1)C(=O)O ; O=C([O-])C(F)(F)F] | (400 MHz, D₂O) 10.30 (s, 1H), 10.19 (d, 1H), 9.90 (d, 1H), 9.18 (dd, 1H), 5.32 (t, 2H), 3.99- 4.21 (m, 3H), 1.34 (d, 3H) (NH and CO₂H protons missing) |
| A23 | 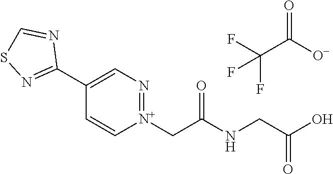 [SMILES: O=C(O)CNC(=O)C[n+]1ccc(-c2ncsn2)cn1 ; O=C([O-])C(F)(F)F] | (400 MHz, D₂O) 10.36 (s, 1H), 10.23 (d, 1H), 9.88 (d, 1H), 9.29 (dd, 1H), 5.84 (s, 2H), 4.05 (s, 2H) (NH and CO₂H protons missing) |
| A24 | 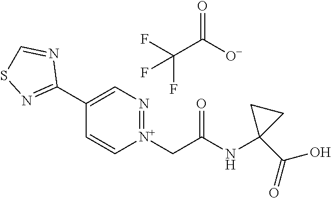 [SMILES: O=C(C[n+]1ccc(-c2ncsn2)cn1)NC1(C(=O)O)CC1 ; O=C([O-])C(F)(F)F] | (400 MHz, D₂O) 10.33 (s, 1H), 10.19 (d, 1H), 9.84 (d, 1H), 9.26 (dd, 1H), 5.75 (s, 2H), 1.49- 1.60 (m, 2H), 1.18-1.26 (m, 2H) (NH and CO₂H protons missing) |

### Biological Examples

### Post-Emergence Efficacy

Seeds of a variety of test species were sown in standard soil in pots. After cultivation for 14 days (post-emergence) under controlled conditions in a glasshouse (at 24/16° C., day/night; 14 hours light; 65% humidity), the plants were sprayed with an aqueous spray solution derived from the dissolution of the technical active ingredient formula (I) in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted to required concentration using 0.25% or 1% Empicol ESC70 (Sodium lauryl ether sulphate)+1% ammonium sulphate as diluent.

The test plants were then grown in a glasshouse under controlled conditions (at 24/16° C., day/night; 14 hours light; 65% humidity) and watered twice daily. After 13 days the test was evaluated (100=total damage to plant; 0=no damage to plant). The results are shown below in Table B1.

### Test Plants:

Ipomoea hederacea (IPOHE), Euphorbia heterophylla (EPHHL), Chenopodium album (CHEAL), Amaranthus palmeri (AMAPA), Lolium perenne (LOLPE), Digitaria sanguinalis (DIGSA), Eleusine indica (ELEIN), Echinochloa crus-galli (ECHCG), Setaria faberi (SETFA)

TABLE B1
Poste-emergence efficacy of Compounds of Formula (I)

| Compound | Application | | | | | | | | | |
|---|---|---|---|---|---|---|---|---|---|---|
| Number | Rate g/Ha | AMAPA | CHEAL | EPHHL | IPOHE | ELEIN | LOLPE | DIGSA | SETFA | ECHCG |
| A2 | 500 | 100 | 70 | 50 | 40 | 80 | 40 | 100 | 100 | 100 |
| A3 | 500 | 100 | 100 | 50 | 40 | 90 | 30 | 100 | 100 | 100 |
| A5 | 500 | 90 | 80 | 60 | 20 | 60 | 50 | 90 | 90 | 100 |
| A6 | 500 | 90 | 80 | 70 | 30 | 80 | 20 | 90 | 80 | 90 |
| A7 | 500 | 100 | 90 | 70 | 20 | 90 | 70 | 100 | 100 | 100 |
| A8 | 500 | 100 | 90 | 70 | 30 | 100 | 70 | 100 | 100 | 100 |
| A9 | 500 | 100 | 50 | 80 | 20 | 70 | 30 | 40 | 40 | 30 |
| A10 | 500 | 100 | 90 | 90 | 60 | 40 | 20 | 40 | 40 | 60 |
| A11 | 500 | 90 | 20 | 90 | 20 | 50 | 30 | 40 | 50 | 50 |
| A12 | 500 | 100 | 90 | 100 | 70 | 70 | 50 | 40 | 50 | 90 |
| A13 | 500 | 100 | 100 | 90 | 60 | 90 | 60 | 100 | 100 | 100 |
| A14 | 500 | 100 | 90 | 100 | 70 | 100 | 50 | 100 | 90 | 100 |
| A15 | 500 | 100 | 100 | 50 | 40 | 90 | 30 | 100 | 100 | 100 |
| A16 | 500 | 100 | 90 | 40 | 20 | 100 | 60 | 100 | 100 | 100 |
| A17 | 500 | 0 | 30 | 20 | 10 | 10 | 0 | 80 | 20 | 20 |
| A18 | 500 | 30 | 50 | 10 | 0 | 0 | 0 | 0 | 20 | 10 |
| A19 | 500 | 100 | 90 | 90 | 50 | 80 | 40 | 80 | 90 | 80 |
| A20 | 500 | 100 | 100 | 80 | 60 | 90 | 90 | 100 | 100 | 100 |
| A21 | 500 | 100 | 90 | 80 | 70 | 40 | 40 | 30 | 100 | 30 |
| A22 | 500 | 100 | 50 | 20 | 50 | 100 | 60 | 100 | 100 | 80 |
| A23 | 500 | 100 | 100 | 90 | 90 | 70 | 50 | 40 | 80 | 50 |
| A24 | 500 | 100 | 90 | 90 | 90 | 70 | 50 | 100 | 80 | 80 |

1. A compound of formula (I) or an agronomically acceptable salt or zwitterionic species thereof: 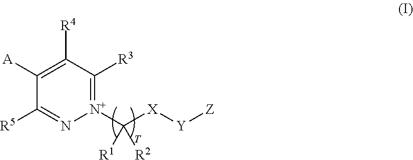
wherein
T is 1, 2 or 3;
R¹ and R² are independently selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
provided that when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, then the R² on the same carbon atom is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Y is (CR^{1a}R^{2b})ₘ;
m is 1, 2 or 3;
each R^{1a} is independently selected from the group consisting of hydrogen, halogen, C₁-C₆alkyl, C₂-C₆alkenyl, C₂-C₆alkynyl, C₃-C₆cycloalkyl, C₁-C₆haloalkyl, —OH, —OR⁷, —OR^{15a}, —NH₂, —NHR⁷, —N(R⁷)₂, —NHR^{15a}, —NR^{7b}R^{7c}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂, —S(O)ᵣR¹⁵ and phenyl which is optionally substituted by 1, 2 or 3 R⁹ substituents, which may be the same or different, —C₁-C₆alkylNH₂, —C₁-C₆alkylNHR⁷, —C₁-C₆alkylN(R⁷)₂, —C₁-C₆alkylC(O)OR¹⁰, —C₁-C₆alkylOR¹⁰, —C₁-C₆alkylC(O)NR¹⁶R¹⁷, —C₁-C₆alkylSR¹⁰, —C₁-C₆alkylS(O)R¹⁰, —C₁-C₆alkylS(O)₂R¹⁰, —C₁-C₆NHC(═NH)NH₂, —C₁-C₃alkylphenyl, wherein said phenyl is optionally substituted by 1, 2 or 3 R⁹ substituents, which may be the same or different, and —C₁-C₃alkylheteroaromatic, wherein said heteroaromatic is a 5- to 10-membered cyclic or bicyclic aromatic ring which comprises 1, 2, 3 or 4 heteroatoms individually selected from nitrogen, oxygen and sulfur, optionally substituted by 1, 2 or 3 R⁹ substituents, which may be the same or different;
each R^{2b} is independently selected from the group consisting of hydrogen, halogen, C₁-C₆alkyl, C₁-C₆haloalkyl, —C₁-C₆alkylNH₂, —C₁-C₆alkylNHR⁷, —C₁-C₆alkylN(R⁷)₂, —C₁-C₆alkylC(O)OR¹⁰, —C₁-C₆alkylOR¹⁰, —C₁-C₆alkylC(O)NR¹⁶R¹⁷, —C₁-C₆alkylSR¹⁰, —C₁-C₆alkylS(O)R¹⁰, —C₁-C₆alkylS(O)₂R¹⁰, —C₁-C₆NHC(═NH)NH₂, —C₁-C₃alkylphenyl, wherein said phenyl is optionally substituted by 1, 2 or 3 R⁹ substituents, which may be the same or different, and —C₁-C₃alkylheteroaromatic, wherein said heteroaromatic is a 5- to 10-membered cyclic or bicyclic aromatic ring which comprises 1, 2, 3 or 4 heteroatoms individually selected from nitrogen, oxygen and sulfur, optionally substituted by 1, 2 or 3 R⁹ substituents, which may be the same or different; or
R^{1a} and R^{2b} together with the carbon atom to which they are attached form a C₃-C₆cycloalkyl ring or a 3- to 6-membered heterocyclyl, which comprises 1 or 2 heteroatoms individually selected from N and O;
R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;
each R⁶ is independently selected from hydrogen and C₁-C₆alkyl;
each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵ —C(O)NR¹⁶R¹⁷ and —C(O)NR⁶R^{15a}a;
R^{7b} and R^{7c} are independently selected from the group consisting of C₁-C₆alkyl, —S(O)ᵣR¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S;
A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different,
and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different;
and/or
when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy; and
each R⁹ is independently selected from the group consisting of OH, halogen, cyano, —N(R⁶)₂, C₁-C₄alkyl, C₁-C₄alkoxy, C₁-C₄haloalkyl and C₁-C₄haloalkoxy;
X is selected from the group consisting of —C(O)—, —C(O)O—, —C(O)N(R⁴⁰)—, —C(O)N(R⁴²)O—, —C(O)N(R⁴⁰)N(R⁴⁰)—, —C(O)N(R⁴⁰)C(O)—, —C(O)N(R⁴⁰)C(O)N(R⁴⁰)—, —C(O)N(R⁴⁰)C(R⁴⁶)₂C(O)N(R⁴⁰)—, —C(O)N(R⁴⁰)C(R⁴⁶)₂C(O)N(R⁴⁰)C(R⁴⁶)₂C(O)N(R⁴⁰)—, —C(═NR⁴¹)—, —C(R⁴⁰)═NO—, —C(═NR⁴¹)N(R⁴⁰)—, —C(S)—, —C(S)N(R⁴⁰)—, —N(R⁴³)—, —N(R⁴²)O—, —N(R⁴³)N(R⁴³)—, —N(R⁴⁰)C(O)—, —N(R⁴⁰)C(S)—, —N(R⁴⁰)S(O)₂—, —N(R⁴⁰)C(O)O—, —N(R⁴⁰)P(O)(R⁴⁴)—, —N(R⁴⁰)P(O)(R⁴⁴)O—, —N(R⁴⁰)C(═NR⁴¹)—, —N(R⁴⁰)S(O)(═NR⁴⁰)—, —N(R⁴⁰)S(O)—, —N(R⁴⁰)C(O)S—, —N(R⁴⁰)C(O)N(R⁴⁰)—, —N(R⁴⁰)S(O)₂N(R⁴⁰)—, —N(R⁴⁰)C(S)N(R⁴⁰)—, —N(R⁴⁰)C(═NR⁴¹)N(R⁴⁰)—, —N(R⁴⁰)P(O)(R⁴⁴)N(R⁴⁰)—, —N(R⁴⁰)C(O)N(R⁴⁰)C(O)—, —N(R⁴⁰)N(R⁴⁰)C(O)—, —O—, —OC(O)—, —OC(O)O—, —OC(O)N(R⁴⁰)—, —ON(R⁴²)—, —ON═C(R⁴⁰)—, —ON(R⁴²)C(O)—, —OP(O)(R⁴⁴)—, —OP(O)(R⁴⁴)O—, —OP(O)(R⁴⁴)N(R⁴⁰)—, —OSi(R⁴⁰)₂—, —OSi(R⁴⁰)₂O—, —S—, —S(O)—, —S(O)₂—, —S(O)₂N(R⁴⁰)—, —SC(O)N(R⁴⁰)—, —S(O)N(R⁴⁰)—, —S(O)(═NR⁴⁰)—, —S(═NR⁴⁰)₂—, —S(O)(═NR⁴⁰)N(R⁴⁰)—, —S(═NR⁴⁰)—, —P(O)(R⁴⁴)—, —P(O)(R⁴⁴)N(R⁴⁰)—, —P(O)(R⁴⁴)O—, —C(═CR⁴⁵)₂—, —CR⁴⁵═CR⁴⁵— (E and Z isomers), —C≡C—, —Si(R⁴⁰)₂— and —Si(R⁴⁰)₂O—;
R⁴⁰ is selected from the group consisting of hydrogen, C₁-C₆alkyl, C₁-C₆alkoxy, C₁-C₃alkoxyC₁-C₃alkyl;
R⁴¹ is selected from the group consisting of hydrogen, C₁-C₆alkyl, C₁-C₆alkoxy, C₁-C₆alkylamino, di-C₁-C₆alkylamino, cyano;
R⁴² is selected from the group consisting of hydrogen, C₁-C₆alkyl, C₁-C₆alkoxyC₁-C₃alkyl, C₁-C₆ alkylcarbonyl, C₁-C₆alkoxycarbonyl, C₁-C₆alkylsulfonyl;
R⁴³ is selected from the group consisting of hydrogen, C₁-C₆alkyl, C₁-C₆alkoxy, C₁-C₃alkoxyC₁-C₃alkyl, C₁-C₆alkylcarbonyl, C₁-C₆alkoxycarbonyl, and C₁-C₆alkylsulfonyl;
R⁴⁴ is selected from the group consisting of hydrogen, C₁-C₆alkyl, OH, C₁-C₆alkoxy, C₁-C₆alkoxyC₁-C₃alkyl, NH₂, and C₁-C₆alkylamino, di-C₁-C₆alkylamino,
R⁴⁵ is selected from the group consisting of hydrogen, halogen, and C₁-C₆alkyl;
R⁴⁶ is selected from the group consisting of hydrogen, C₁-C₆alkyl, C₁-C₆alkoxy, C₁-C₆alkoxyC₁-C₃alkyl, —C₁-C₆alkylNH₂, —C₁-C₆alkylNHR⁷, —C₁-C₆alkylN(R⁷)₂, —C₁-C₆alkylC(O)OR¹⁰, —C₁-C₆alkylOR¹⁰, —C₁-C₆alkylC(O)NR¹⁶R¹⁷, —C₁-C₆alkylSR¹⁰, —C₁-C₆alkylS(O)R¹⁰, —C₁-C₆alkylS(O)₂R¹⁰, —C₁-C₆NHC(═NH)NH₂, —C₁-C₃alkylC₁-C₃alkoxy, —C₁-C₃alkylphenyl, wherein said phenyl is optionally substituted by 1, 2 or 3 R⁹ substituents, which may be the same or different, and —C₁-C₃alkylheteroaromatic, wherein said heteroaromatic is a 5- to 10-membered cyclic or bicyclic aromatic ring which comprises 1, 2, 3 or 4 heteroatoms individually selected from nitrogen, oxygen and sulfur, optionally substituted by 1, 2 or 3 R⁹ substituents, which may be the same or different;
Z is selected from the group consisting of —C(O)OR¹⁰, —OH, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S;
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2.

2. The compound according to claim 1 wherein T is 1.

3. The compound according to claim 1, wherein R¹ and R² are independently selected from the group consisting of hydrogen and C₁-C₆alkyl.

4. The compound according to claim 1, wherein m is 1 or 2.

5. The compound according to claim 1, wherein R³, R⁴ and R⁵ are independently selected from the group consisting of hydrogen and methyl.

6. The compound of formula (I) according to claim 1, wherein A is selected from the group consisting of formula A-I to A-XXXII below 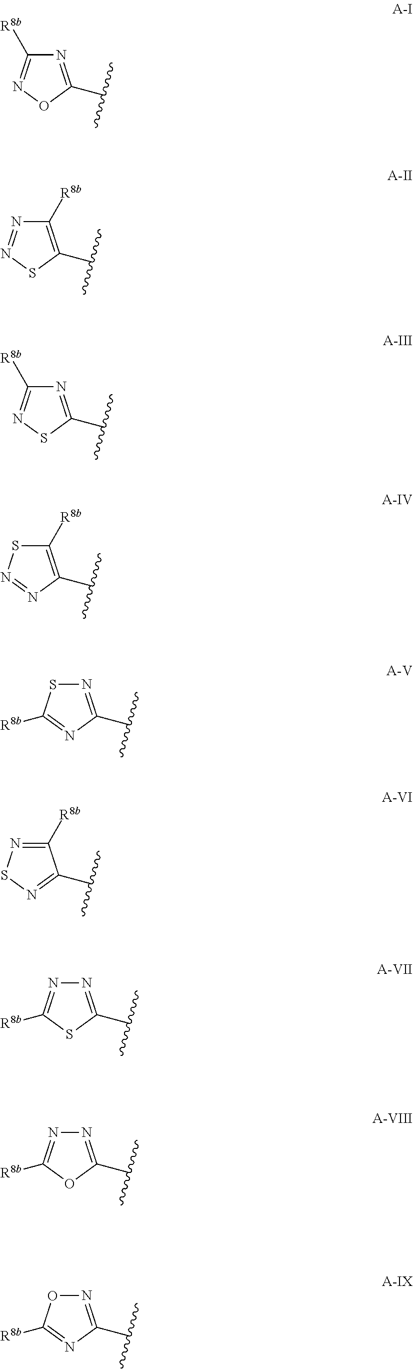 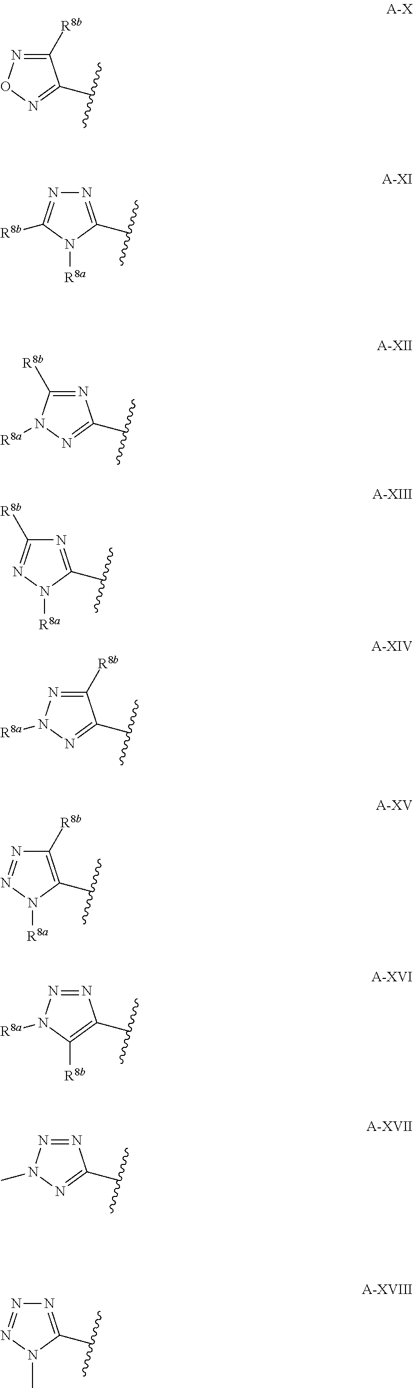 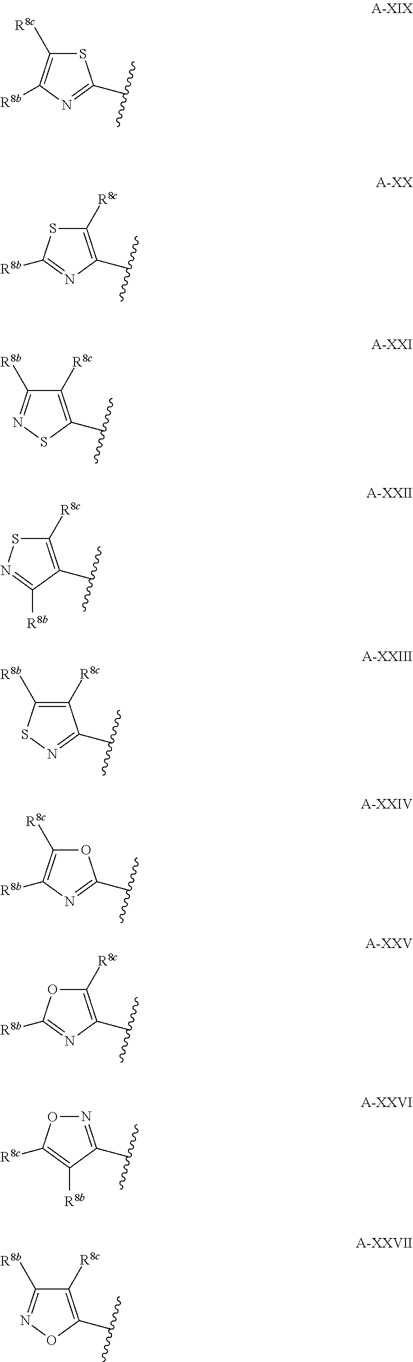 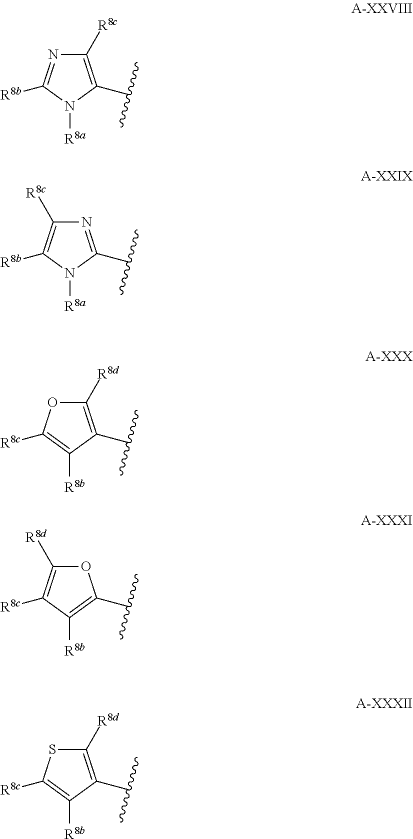
wherein the jagged line defines the point of attachment to a compound of formula (I);
R^{8a} is selected from the group consisting of hydrogen, C₁-C₆alkyl and C₁-C₆haloalkyl;
each R^{8b}, R^{8c} and R^{8d} are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl;
and R¹⁰, R¹⁵, R¹⁶, R¹⁷ and r are as defined in claim 1.

7. The compound of formula (I) according to claim 1, wherein A is selected from the group consisting of formula A-Ia to A-VIIIa below

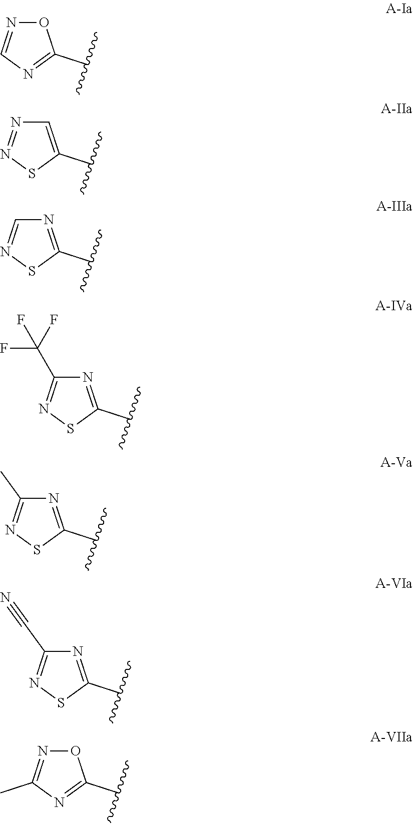

8. The compound according to claim 1, wherein A is selected from the group consisting of formula A-I to A-III below 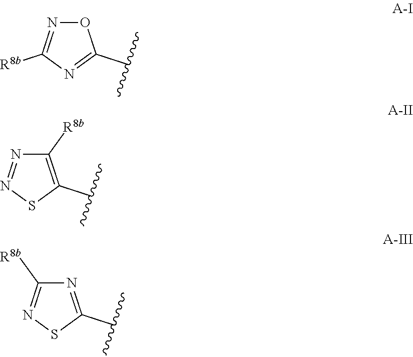
wherein the jagged line defines the point of attachment to a compound of formula (I), and each R^{8b} and R¹⁶ and R¹⁷ are as defined herein.

9. The compound according to claim 1, wherein A is substituted with 1 or 2 R⁸ and each R⁸ is independently selected from the group consisting of chloro, fluoro, cyano, —NH₂, —NMe₂, —OMe, —S(O)₂Me, —C(O)NHMe, —C(O)NMe₂, methyl and trifluoromethyl.

10. The compound according to claim 1, wherein wherein A is substituted with 3 or 4 R⁸ and each R⁸ is independently selected from the group consisting of C₁-C₆alkyl and C₁-C₆haloalkyl.

11. The compound according to claim 1 wherein X is selected from the group consisting of —C(O)N(R⁴⁰)—, —S(O)—, —S(O)₂— and —S(O)₂N(R⁴⁰)— and R⁴⁰ is selected from hydrogen and C₁-C₆alkyl.

12. The compound according to claim 1, wherein Z is selected from the group consisting of —C(O)OR¹⁰, —C(O)NHS(O)₂R¹², —S(O)₂OR¹⁰, and —P(O)(R¹³)(OR¹⁰).

13. The compound according to claim 12, wherein Z is —C(O)OH or —S(O)₂OH.

14. An agrochemical composition comprising a herbicidally effective amount of a compound of formula (I) as defined in claim 1.

15. The composition according to claim 14, further comprising at least one additional active ingredient and/or an agrochemically-acceptable diluent or carrier.

16. A method of controlling unwanted plant growth, comprising applying a compound of formula (I) as defined in claim 1, or a herbicidal composition according to claim 14, to the unwanted plants or to the locus thereof.
